(19) United States
(12) Patent Application Publication
(10) Pub. No.: US 2018/0087079 A1
(43) Pub. Date: Mar. 29, 2018

(54) METHODS OF USING CELL-CYCLE INHIBITORS TO MODULATE ONE OR MORE PROPERTIES OF A CELL CULTURE

(71) Applicant: AMGEN INC., Thousand Oaks, CA (US)

(72) Inventors: Zhimei DU, Bellevue, WA (US); John Douglas MCCARTER, Ventura, CA (US); Pranhitha REDDY, Seattle, WA (US); Andrew William SNOWDEN, Germantown, MD (US); Lawrence R. MCGEE, Pacifica, CA (US); John Gordon ALLEN, Newbury Park, CA (US); David Lawrence TREIBER, North Bend, WA (US); Kathleen KEEGAN, Bainbridge Island, WA (US); Zhihong LI, Millbrae, CA (US)

(73) Assignee: AMGEN INC., Thousand Oaks, CA (US)

(21) Appl. No.: 15/828,215

(22) Filed: Nov. 30, 2017

Related U.S. Application Data
(63) Continuation of application No. 14/760,978, filed on Jul. 14, 2015, now abandoned.
(60) Provisional application No. 61/735,733, filed on Jan. 14, 2013.

(51) Int. Cl. C12P 21/00; C07D 471/04; C07K 16/00

(52) U.S. Cl. CPC C12P 21/00; C07D 471/04; C07K 16/00

(57) ABSTRACT

Methods of modulating the properties of a cell culture expressing a protein of interest are provided. In various embodiments the methods relate to the addition of cell cycle inhibitors to growing cell cultures.

### CROSS REFERENCE TO RELATED APPLICATIONS

This application claims the benefit under 35 U.S.C. § 119 of U.S. Provisional Application Ser. No. 61/735,733, filed Jan. 14, 2013 which is hereby incorporated by reference.

### FIELD OF THE INVENTION

The present invention relates generally to compounds and processes for modulating one or more properties of a cell culture, including mammalian cell cultures such as CHO cell cultures.

### BACKGROUND OF THE INVENTION

The need for greater quantities of therapeutic recombinant proteins has resulted in investigation of various methods to increase recombinant protein production, including attempts to improve cell development, optimize media formulation and implement process control parameters. Process optimization is an area that is of increasing interest, particularly as it relates to methods and strategies for growing, feeding, and maintaining production cell cultures.

Improvements to cell culture processes, recombinant polypeptide expression, titer, and cell viability can lead to higher production levels, thereby reducing the costs associated with manufacturing protein therapeutics. One aspect of the cell culture process that has been an area of investigation is increasing specific productivity by inducing cell cycle G1 arrest. The cell cycle is the process by which a single cell divides to form new cells. As part of the normal cycle, cells move thorough four phases, the G1, or first gap, phase, the S, or synthesis, phase, the G2, or second gap, phase and the M phase during which mitosis, or cell division, occurs. Biosynthetic activity occurs at a high rate during the G1 phase; this activity is followed by the commencement of DNA synthesis in preparation for cell division. The G2 phase is also a time of increased biosynthetic activity, most of which activity is dedicated to production of proteins necessary for mitosis.

While it is necessary for cultured cells that are producing a recombinant protein to cycle through these various phases, it is helpful to be able to control the amount of time the cultured cells spend in each phase. For example, it is needful for cultured cells to undergo a robust and complete cell cycle in order to increase the number of cells in the early stages of a production culture, but when a desired cell density has been reached, it is desirable to maintain the cells in a stage of the cell cycle that facilitates production of recombinant protein without overwhelming the culture process with sheer numbers of cells, which can lead to problems with both manufacturing process control and harvest.

Numerous aspects of the cell culture process and their effects on the cell growth cycle have been investigated, including limitation of key nutrients such as amino acids; temperature shifts; and inclusion of various chemicals that modulate one or more aspects of cell growth. Despite these varied approaches, control of cell growth is still a challenge in the cell culture process, with a resultant need for alternative methods of controlling the cell cycle in manufacture of proteins in large quantities. The invention fulfills these needs by providing a method of controlling cell growth while increasing protein production

### SUMMARY OF THE INVENTION

In one embodiment of the invention, there is provided a method of increasing specific productivity in a mammalian cell culture expressing a recombinant protein comprising establishing a mammalian cell culture in a culture medium; inducing cell growth-arrest by contacting the cell culture with a culture medium comprising a cell cycle inhibitor; and maintaining the cell culture in a growth-arrested state by contacting the culture with a culture medium comprising a cell cycle inhibitor. In another embodiment, the invention provides a method of increasing recombinant protein production in a mammalian cell culture expressing a recombinant protein comprising establishing a mammalian cell culture in a culture medium; inducing cell growth-arrest by contacting the cell culture with a culture medium comprising a cell cycle inhibitor; and maintaining the cell culture in a growth-arrested state by contacting the culture with a culture medium comprising a cell cycle inhibitor. Further provided is a method of limiting a mammalian cell culture expressing a recombinant protein at a desired packed cell volume comprising establishing a mammalian cell culture in a culture medium; inducing cell growth-arrest by contacting the cell culture with a culture medium comprising a cell cycle inhibitor; and maintaining the cell culture in a growth-arrested state by contacting the culture with a culture medium comprising a cell cycle inhibitor.

In any of the foregoing methods, the cell culture is contacted with culture medium comprising a cell cycle inhibitor on or before day 3 of the culture. In a further embodiment, induction of cell growth-arrest takes place prior to a production phase. In a still further embodiment, induction of cell growth-arrest takes place during a production phase. Additionally, a further embodiment of the invention provides that in any of the embodiments discussed herein, the cell cycle inhibitor is a CDK4 inhibitor.

Additional embodiments of the invention include the methods previously described, wherein the cell culture is grown by a method selected from the group consisting of batch culture, fed-batch culture, perfusion culture, and combinations thereof. Further embodiments comprise a temperature shift from 36° C. to 31° C.; additional embodiments further comprise limitation of a key nutrient in the culture medium.

The invention also provides a method of culturing mammalian cells expressing a recombinant protein comprising; establishing a mammalian cell culture in a culture medium; growing the mammalian cells during a growth phase and supplementing the culture medium with bolus feeds of a culture medium, and maintaining the mammalian cells during a production phase by perfusion with a culture medium (for example, a serum free perfusion medium) comprising a cell cycle inhibitor, wherein the packed cell volume during the production phase is less than or equal to 35%. In one embodiment, perfusion begins on or about day 5 to on or about day 9 of the cell culture; in another embodiment, perfusion begins on or about day 5 to on or about day 7 of the cell culture. Further embodiments include those in which perfusion begins when the cells have reached a production phase.

In one embodiment of the present inventive methods, the cell cycle inhibitor is a CDK4 inhibitor; in further embodiments, the concentration of cell cycle inhibitor in the culture medium is less than or equal to 5 microM; less than or equal to 4.0 microM; less than or equal to 3.0 microM, less than or equal to 2.0 microM; or less than or equal to 1.0 microM. Still further embodiments include use of a cell cycle inhibitor, optionally a CDK4 inhibitor, wherein the concentration of cell cycle inhibitor in the culture medium is between 0.5 and 5 microM, or between 1 and 4.0 microM, or between 2 and 3.0 microM. In certain embodiments, the concentration of cell cycle inhibitor in the culture medium is between 2.5 and 5 microM, or between 3.5 and 5 microM. In other embodiments, the concentration is between 5 microM and 10 microM, between 6 microM and 9 microM, between 7 microM and 8 microM, between 7.5 and 10 microM, or between 9.5 and 10.5 microM. Further embodiments include use of cell cycle inhibitors at 0.625, 1.2, 1.25, 2.4, 2.5, 5.0, 10.0, 20.0, 2530 microM, as well as similar concentrations.

Further embodiments include variations of the previously described methods, wherein the packed cell volume during a production phase is less than or equal to 35%, or is less than or equal to 30%. Additional embodiments utilize a viable cell density of the mammalian cell culture at a packed cell volume less than or equal to 35% is 10×10⁶ viable cells/ml to 80×10⁶ viable cells/ml, or 20×10⁶ viable cells/ml to 30×10⁶ viable cells/ml.

In certain embodiments, the methods comprise continuous perfusion. In one embodiment, the rate of perfusion is constant; in another, perfusion is performed at a rate of less than or equal to 1.0 working volumes per day. For some of the inventive methods, perfusion is performed at a rate that increases during the production phase from 0.25 working volume per day to 1.0 working volume per day during the cell culture, or perfusion is performed at a rate that reaches 1.0 working volume per day on day 9 to day 11 of the cell culture or at a rate that reaches 1.0 working volume per day on day 10 of the cell culture.

The invention also provides methods in which the bolus feeds of culture medium begin on day 3 or day 4 of the cell culture. In one embodiment, the mammalian cell culture is established by inoculating a bioreactor with between 0.5×10⁶ and 3.0×10⁶ cells/mL in a culture medium, or between 0.5×10⁶ and 1.5×10⁶ cells/mL in a culture medium. Further embodiments comprise a temperature shift from 36° C. to 31° C., or from 36° C. to 33° C., in the inventive methods. In another embodiment, the mammalian cell culture is established by inoculating a bioreactor with at least 0.5×10⁶ cells/mL in a culture medium, or at least 1.5×10⁶ cells/mL in a culture medium. Further embodiments comprise a temperature shift from 36° C. to 31° C., or from 36° C. to 33° C., in the inventive methods.

As described herein, one aspect of the inventive methods utilizes perfusion that is accomplished by alternating tangential flow. Bioreactors can be used for the inventive methods, for example, a bioreactor that has a capacity of at least 500 L, between 500 L and 2000 L, or between 1000 L and 2000 L. In further embodiments, the bioreactor has a capacity of at least 100 L, at least 200 L, at least 500 L, at least 1000 L or at least 2000 L.

Additional embodiments of the inventive methods can be applied to various cells, for example, Chinese Hamster Ovary (CHO) cells. The inventive methods can be used to produce recombinant proteins wherein the recombinant protein is selected from the group consisting of a human antibody, a humanized antibody, a chimeric antibody, a recombinant fusion protein, or a cytokine. Additional embodiments of the present inventive methods further comprise a step of harvesting the recombinant protein produced by the cell culture; in further embodiments, the recombinant protein produced by the cell culture is purified and formulated in a pharmaceutically acceptable formulation.

### BRIEF DESCRIPTION OF THE DRAWINGS

FIG. 1 is a representative FACS readout demonstrating the presence of cells in G1, S and G2 phases of the cell growth cycle. Panel A presents results obtained with CHO cells grown in the absence of a cell cycle inhibitor; panel B presents results obtained with CHO cells grown in the presence of a cell cycle inhibitor.

FIG. 2 presents an analysis of the effects of a cell cycle inhibitor on viable cell density (VCD; expressed in 10⁶ cells/mL, Panel A); percent viability (Panel B); titer of recombinant protein (expressed in g/L; Panel C); qP, or specific productivity (expressed in pg/c/d; Panel D), the presence or absence of high molecular weight substances (expressed as % of the total protein; Panel E); and the relative percentage of Mannose 5 (expressed as % of protein exhibiting high mannose levels; Panel F), essentially as described in Example 3. In each panel, the closed diamonds represent results obtained in the absence of a cell cycle inhibitor, the closed squares represents results obtained with the inclusion of a cell cycle inhibitor at 5 microM), and the closed triangles represent results obtained with the inclusion of a cell cycle inhibitor at 10 microM.

FIG. 3 illustrates the effects of a cell cycle inhibitor (added at day 8) on cells grown in a 2 L bioreactor. Parameters evaluated were viable cell density (VCD; expressed in 10⁶ cells/mL, Panel A); percent viability (Panel B); titer of recombinant protein (expressed in g/L; Panel C); qP, or specific productivity (expressed in pg/c/d; Panel D), the presence or absence of high molecular weight substances (expressed as % of the total protein; Panel E); and the relative percentage of Mannose 5 (expressed as % of protein exhibiting high mannose levels; Panel F). In each panel, the closed diamonds represent results obtained in the absence of a cell cycle inhibitor, the closed squares represents results obtained with the inclusion of a cell cycle inhibitor at 5 microM), and the closed triangles represent results obtained with the inclusion of a cell cycle inhibitor at 10 microM.

### DETAILED DESCRIPTION OF THE INVENTION

While the terminology used in this application is standard within the art, definitions of certain terms are provided herein to assure clarity and definiteness in the meaning of the claims. Units, prefixes, and symbols may be denoted in their SI (International System of Units) accepted form. Numeric ranges recited herein are inclusive of the numbers defining the range and include and are supportive of each integer within the defined range. The methods and techniques described herein are generally performed according to conventional methods well known in the art and as described in various general and more specific references that are cited and discussed throughout the present specification unless otherwise indicated. See, e.g., Sambrook et al. Molecular Cloning: A Laboratory Manual, 3rd ed., Cold Spring Harbor Laboratory Press, Cold Spring Harbor, N.Y. (2001) and Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990).

The disclosed methods are applicable to adherent culture or suspension cultures grown in stirred tank reactors (including traditional batch and fed-batch cell cultures, which may but need not comprise a spin filter), perfusion systems (including alternating tangential flow (“ATF”) cultures, acoustic perfusion systems, depth filter perfusion systems, and other systems), hollow fiber bioreactors (HFB, which in some cases may be employed in perfusion processes) as well as various other cell culture methods (see, e.g., Tao et al., (2003) Biotechnol. Bioeng. 82:751-65; Kuy stermans & Al-Rubeai, (2011) “Bioreactor Sy stems for Producing Antibody from Mammalian Cells” in Antibody Expression and Production, Cell Engineering 7:25-52, Al-Rubeai (ed) Springer; Catapano et al., (2009) “Bioreactor Design and Scale-Up” in Cell and Tissue Reaction Engineering: Principles and Practice, Eibl et al. (eds) Springer-Verlag, incorporated herein by reference in their entireties).

All documents, or portions of documents, cited in this application, including but not limited to patents, patent applications, articles, books, and treatises, are hereby expressly incorporated by reference. What is described in an embodiment of the invention can be combined with other embodiments of the invention.

### Definitions

As used herein, the terms “a” and “an” mean one or more unless specifically indicated otherwise.

The instant disclosure provides methods of modulating the properties of cell cultures expressing a “protein of interest;” as used herein a “protein of interest” includes naturally occurring proteins, recombinant proteins, and engineered proteins (e.g., proteins that do not occur in nature and which have been designed and/or created by humans). A protein of interest can, but need not be, a protein that is known or suspected to be therapeutically relevant. Particular examples of a protein of interest include antigen binding proteins (as described and defined herein), peptibodies (i.e., a molecule comprising peptide(s) fused either directly or indirectly to other molecules such as an Fc domain of an antibody, where the peptide moiety specifically binds to a desired target; the peptide(s) may be fused to either an Fc region or inserted into an Fc-Loop, or a modified Fc molecule, for example as described in U.S. Patent Application Publication No. US2006/0140934 incorporated herein by reference in its entirety), fusion proteins (e.g., Fc fusion proteins, wherein a Fc fragment is fused to a protein or peptide), cytokines, growth factors, hormones and other naturally occurring secreted proteins, as well as mutant forms of naturally occurring proteins.

As used herein, the term “antigen binding protein” is used in its broadest sense and means a protein comprising a portion that binds to an antigen or target and, optionally, a scaffold or framework portion that allows the antigen binding portion to adopt a conformation that promotes binding of the antigen binding protein to the antigen. Examples of antigen binding proteins include a human antibody, a humanized antibody; a chimeric antibody; a recombinant antibody; a single chain antibody; a diabody; a triabody; a tetrabody; a Fab fragment; a F(ab′)2 fragment; an IgD antibody; an IgE antibody; an IgM antibody; an IgG1 antibody; an IgG2 antibody; an IgG3 antibody; or an IgG4 antibody, and fragments thereof. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, e.g., Korndorfer et al., 2003, Proteins: Structure, Function, and Bioinformatics, 53(1):121-129 (2003); Roque et al., Biotechnol. Prog. 20:639-654 (2004). In addition, peptide antibody mimetics (“PAMs”) can be used, as well as scaffolds based on antibody mimetics utilizing fibronectin components as a scaffold.

An antigen binding protein can have, for example, the structure of a naturally occurring immunoglobulin. An “immunoglobulin” is a tetrameric molecule. In a naturally occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively.

Naturally occurring immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4. The assignment of amino acids to each domain can be done in accordance with the definitions of Kabat et al. in Sequences of Proteins of Immunological Interest, 5^{th} Ed., US Dept. of Health and Human Services, PHS, NIH, NIH Publication no. 91-3242, (1991). As desired, the CDRs can also be redefined according an alternative nomenclature scheme, such as that of Chothia (see Chothia & Lesk, (1987) J. Mol. Biol. 196:901-917; Chothia et al., (1989) Nature 342:878-883 or Honegger & Pluckthun, (2001) J. Mol. Biol. 309:657-670).

In the context of the instant disclosure an antigen binding protein is said to “specifically bind” or “selectively bind” its target antigen when the dissociation constant (K_{D}) is ≦10⁻⁸ M. The antibody specifically binds antigen with “high affinity” when the K_{D} is ≦5×10⁻⁹ M, and with “very high affinity” when the K_{D} is ≦5×10⁻¹⁰ M.

The term “antibody” includes reference to both glycosylated and non-glycosylated immunoglobulins of any isotype or subclass or to an antigen-binding region thereof that competes with the intact antibody for specific binding, unless otherwise specified. Additionally, as used herein, the term “antibody” refers to an intact immunoglobulin or to an antigen binding portion thereof that competes with the intact antibody for specific binding, unless otherwise specified. Antigen binding portions can be produced by recombinant DNA techniques or by enzymatic or chemical cleavage of intact antibodies and can form an element of a protein of interest. Antigen binding portions include, inter alia, Fab, Fab′, F(ab′)₂, Fv, domain antibodies (dAbs), fragments including complementarity determining regions (CDRs), single-chain antibodies (scFv), chimeric antibodies, diabodies, triabodies, tetrabodies, and polypeptides that contain at least a portion of an immunoglobulin that is sufficient to confer specific antigen binding to the polypeptide.

A Fab fragment is a monovalent fragment having the V_{L}, V_{H}, C_{L} and C_{H}1 domains; a F(ab′)₂ fragment is a bivalent fragment having two Fab fragments linked by a disulfide bridge at the hinge region; a Fd fragment has the V_{H} and C_{H}1 domains; an Fv fragment has the V_{L} and V_{H} domains of a single arm of an antibody; and a dAb fragment has a V_{H} domain, a V_{L} domain, or an antigen-binding fragment of a V_{H} or V_{L} domain (U.S. Pat. Nos. 6,846,634, 6,696,245, U.S. App. Pub. Nos. 05/0202512, 04/0202995, 04/0038291, 04/0009507, 03/0039958, Ward et al., (1989) Nature 341:544-546).

A single-chain antibody (scFv) is an antibody in which a V_{L} and a V_{H} region are joined via a linker (e.g., a synthetic sequence of amino acid residues) to form a continuous protein chain wherein the linker is long enough to allow the protein chain to fold back on itself and form a monovalent antigen binding site (see, e.g., Bird et al., Science 242:423-26 (1988) and Huston et al., (1988) Proc. Natl. Acad. Sci. USA 85:5879-83). Diabodies are bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., (1993) Proc. Natl. Acad. Sci. USA 90:6444-48; and Poljak et al., (1994) Structure 2:1121-23). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

One or more CDRs can be incorporated into a molecule either covalently or noncovalently to make it an antigen binding protein. An antigen binding protein can incorporate the CDR(s) as part of a larger polypeptide chain, can covalently link the CDR(s) to another polypeptide chain, or can incorporate the CDR(s) noncovalently. The CDRs permit the antigen binding protein to specifically bind to a particular antigen of interest.

An antigen binding protein can have one or more binding sites. If there is more than one binding site, the binding sites can be identical to one another or can be different. For example, a naturally occurring human immunoglobulin typically has two identical binding sites, while a “bispecific” or “bifunctional” antibody has two different binding sites.

For purposes of clarity, and as described herein, it is noted that an antigen binding protein can, but need not, be of human origin (e.g., a human antibody), and in some cases will comprise a non-human protein, for example a rat or murine protein, and in other cases an antigen binding protein can comprise a hybrid of human and non-human proteins (e.g., a humanized antibody).

A protein of interest can comprise a human antibody. The term “human antibody” includes all antibodies that have one or more variable and constant regions derived from human immunoglobulin sequences. In one embodiment, all of the variable and constant domains are derived from human immunoglobulin sequences (a fully human antibody). Such antibodies can be prepared in a variety of ways, including through the immunization with an antigen of interest of a mouse that is genetically modified to express antibodies derived from human heavy and/or light chain-encoding genes, such as a mouse derived from a Xenomouse®, UltiMab™, or Velocimmune® system. Phage-based approaches can also be employed.

Alternatively, a protein of interest can comprise a humanized antibody. A “humanized antibody” has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. Examples of how to make humanized antibodies can be found in U.S. Pat. Nos. 6,054,297, 5,886,152 and 5,877,293.

An “Fc” region, as the term is used herein, comprises two heavy chain fragments comprising the C_{H}2 and C_{H}3 domains of an antibody. The two heavy chain fragments are held together by two or more disulfide bonds and by hydrophobic interactions of the C_{H}3 domains Proteins of interest comprising an Fc region, including antigen binding proteins and Fc fusion proteins, form another aspect of the instant disclosure.

A “hemibody” is an immunologically functional immunoglobulin construct comprising a complete heavy chain, a complete light chain and a second heavy chain Fc region paired with the Fc region of the complete heavy chain A linker can, but need not, be employed to join the heavy chain Fc region and the second heavy chain Fc region. In particular embodiments a hemibody is a monovalent form of an antigen binding protein disclosed herein. In other embodiments, pairs of charged residues can be employed to associate one Fc region with the second Fc region. A hemibody can be a protein of interest in the context of the instant disclosure.

The term “host cell” means a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present. A cell culture can comprise one or more host cells.

As used herein, the term “hybridoma” means a cell or progeny of a cell resulting from fusion of an immortalized cell and an antibody-producing cell. The resulting hybridoma is an immortalized cell that produces antibodies. The individual cells used to create the hybridoma can be from any mammalian source, including, but not limited to, hamster, rat, pig, rabbit, sheep, goat, and human. The term also encompasses trioma cell lines, which result when progeny of heterohybrid myeloma fusions, which are the product of a fusion between human cells and a murine myeloma cell line, are subsequently fused with a plasma cell. The term is meant to include any immortalized hybrid cell line that produces antibodies such as, for example, quadromas (see, e.g., Milstein et al., (1983) Nature, 537:3053).

As used herein, the terms “culture” and “cell culture” are used interchangeably and refer to a cell population that is maintained in a medium under conditions suitable to survival and/or growth of the cell population. As will be clear to those of ordinary skill in the art, these terms as used herein also refer to the combination comprising the cell population and the medium in which the population is suspended.

The terms “polypeptide” and “protein” (e.g., as used in the context of a protein of interest or a polypeptide of interest) are used interchangeably herein to refer to a polymer of amino acid residues. The terms also apply to amino acid polymers in which one or more amino acid residues is an analog or mimetic of a corresponding naturally occurring amino acid, as well as to naturally occurring amino acid polymers. The terms can also encompass amino acid polymers that have been modified, e.g., by the addition of carbohydrate residues to form glycoproteins, or phosphorylated. Polypeptides and proteins can be produced by a naturally-occurring and non-recombinant cell, or polypeptides and proteins can be produced by a genetically-engineered or recombinant cell. Polypeptides and proteins can comprise molecules having the amino acid sequence of a native protein, or molecules having deletions from, additions to, and/or substitutions of one or more amino acids of the native sequence.

The terms “polypeptide” and “protein” encompass molecules comprising only naturally occurring amino acids, as well as molecules that comprise non-naturally occurring amino acids. Examples of non-naturally occurring amino acids (which can be substituted for any naturally-occurring amino acid found in any sequence disclosed herein, as desired) include: 4-hydroxyproline, γ-carboxy glutamate, ε-N,N,N-trimethylly sine, ε-N-acetylly sine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxyl-terminal direction, in accordance with standard usage and convention.

A non-limiting list of examples of non-naturally occurring amino acids that can be inserted into a protein or polypeptide sequence or substituted for a wild-type residue in a protein or polypeptide sequence include β-amino acids, homoamino acids, cyclic amino acids and amino acids with derivatized side chains Examples include (in the L-form or D-form; abbreviated as in parentheses): citrulline (Cit), homocitrulline (hCit), Nα-methylcitrulline (NMeCit), Nα-methylhomocitrulline (Nα-MeHoCit), ornithine (Orn), Nα-Methylornithine (Nα-MeOrn or NMeOrn), sarcosine (Sar), homolysine (hLys or hK), homoarginine (hArg or hR), homoglutamine (hQ), Nα-methylarginine (NMeR), Nα-methylleucine (Nα-MeL or NMeL), N-methylhomolysine (NMeHoK), Nα-methylglutamine (NMeQ), norleucine (Nle), norvaline (Nva), 1,2,3,4-tetrahydroisoquinoline (Tic), Octahydroindole-2-carboxylic acid (Oic), 3-(1-naphthyl)alanine (1-Nal), 3-(2-naphthyl)alanine (2-Nal), 1,2,3,4-tetrahydroisoquinoline (Tic), 2-indanylglycine (IgI), para-iodophenylalanine (pI-Phe), para-aminophenylalanine (4AmP or 4-Amino-Phe), 4-guanidino phenylalanine (Guf), glycyllysine (abbreviated “K(NE-glycyl)” or “K(glycyl)” or “K(gly)”), nitrophenylalanine (nitrophe), aminophenylalanine (aminophe or Amino-Phe), benzylphenylalanine (benzylphe), γ-carboxy glutamic acid (γ-carboxy glu), hydroxyproline (hydroxypro), p-carboxyl-phenylalanine (Cpa), α-aminoadipic acid (Aad), Nα-methyl valine (NMeVal), N-α-methyl leucine (NMeLeu), Nα-methylnorleucine (NMeNle), cyclopentylglycine (Cpg), cyclohexylglycine (Chg), acetylarginine (acetylarg), α,β-diaminopropionoic acid (Dpr), α,γ-diaminobutyric acid (Dab), diaminopropionic acid (Dap), cyclohexylalanine (Cha), 4-methyl-phenylalanine (MePhe), β, β-diphenyl-alanine (BiPhA), aminobutyric acid (Abu), 4-phenyl-phenylalanine (or biphenylalanine; 4Bip), α-amino-isobutyric acid (Aib), beta-alanine, beta-aminopropionic acid, piperidinic acid, aminocaprioic acid, aminoheptanoic acid, aminopimelic acid, desmosine, diaminopimelic acid, N-ethylglycine, N-ethylaspargine, hydroxylysine, allo-hydroxylysine, isodesmosine, allo-isoleucine, N-methylglycine, N-methylisoleucine, N-methylvaline, 4-hydroxyproline (Hyp), γ-carboxyglutamate, α-N,N,N-trimethyllysine, α-N-acetyllysine, O-phosphoserine, N-acetylserine, N-formylmethionine, 3-methylhistidine, 5-hydroxylysine, ω-methylarginine, 4-Amino-O-Phthalic Acid (4APA), and other similar amino acids, and derivatized forms of any of those specifically listed.

By “cell culture” or “culture” is meant the growth and propagation of cells outside of a multicellular organism or tissue. Suitable culture conditions for mammalian cells are known in the art. See e g. Animal cell culture: A Practical Approach, D. Rickwood, ed., Oxford University Press, New York (1992) Mammalian cells may be cultured in suspension or while attached to a solid substrate. Fluidized bed bioreactors, hollow fiber bioreactors, roller bottles, shake flasks, or stirred tank bioreactors, with or without microcarriers, can be used. In one embodiment 500 L to 2000 L bioreactors are used. In one embodiment, 1000 L to 2000 L bioreactors are used.

As used herein, the terms “medium,” “cell culture medium” and “culture medium” are used interchangeably and mean a solution containing nutrients that nourish growing cells. In certain embodiments, a culture medium is useful for growing mammalian cells. Cell culture media formulations are well known in the art; typically, a culture medium provides essential and non-essential amino acids, vitamins, energy sources, lipids, and trace elements required by the cell for minimal growth and/or survival, as well as buffers, and salts. A culture medium may also contain supplementary components that enhance growth and/or survival above the minimal rate, including, but not limited to, hormones and/or other growth factors, particular ions (such as sodium, chloride, calcium, magnesium, and phosphate), buffers, vitamins, nucleosides or nucleotides, trace elements (inorganic compounds usually present at very low final concentrations), amino acids, lipids, and/or glucose or other energy source; as described herein, cell cycle inhibitors can be added to a culture medium. In certain embodiments, a medium is advantageously formulated to a pH and salt concentration optimal for cell survival and proliferation. In certain embodiments, the medium is a culture medium (for example, a feed medium) that is added after the beginning of the cell culture. In certain embodiments, the cell culture medium is a mixture of a starting nutrient solution and any culture medium that is added after the beginning of the cell culture.

“Serum-free” applies to a cell culture medium that does not contain animal sera, such as fetal bovine serum. Various tissue culture media, including defined culture media, are commercially available, for example, any one or a combination of the following cell culture media can be used: RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL′ 300 Series (JRH Biosciences, Lenexa, Kans.), among others. Serum-free versions of such culture media are also available. Cell culture media may be supplemented with additional or increased concentrations of components such as amino acids, salts, sugars, vitamins, hormones, growth factors, buffers, antibiotics, lipids, trace elements and the like, depending on the requirements of the cells to be cultured and/or the desired cell culture parameters.

As used herein, the term “bioreactor” means any vessel useful for the growth of a cell culture. The cell cultures of the instant disclosure can be grown in a bioreactor, which can be selected based on the application of a protein of interest that is produced by cells growing in the bioreactor. A bioreactor can be of any size so long as it is useful for the culturing of cells; typically, a bioreactor is sized appropriate to the volume of cell culture being grown inside of it. Typically, a bioreactor will be at least 1 liter and may be 2, 5, 10, 50, 100, 200, 250, 500, 1,000, 1500, 2000, 2,500, 5,000, 8,000, 10,000, 12,000 liters or more, or any volume in between. The internal conditions of the bioreactor, including, but not limited to pH and temperature, can be controlled during the culturing period. Those of ordinary skill in the art will be aware of, and will be able to select, suitable bioreactors for use in practicing the present invention based on the relevant considerations.

As used herein, “cell density” refers to the number of cells in a given volume of culture medium. “Viable cell density” refers to the number of live cells in a given volume of culture medium, as determined by standard viability assays (such as trypan blue dye exclusion method).

As used herein, the term “cell viability” means the ability of cells in culture to survive under a given set of culture conditions or experimental variations. The term as used herein also refers to that portion of cells which are alive at a particular time in relation to the total number of cells, living and dead, in the culture at that time.

As used herein, “packed cell volume” (PCV), also referred to as “percent packed cell volume” (% PCV), is the ratio of the volume occupied by the cells, to the total volume of cell culture, expressed as a percentage (see Stealer, wt al., (2006) Biotechnol Bioeng. Dec 20:95(6):1228-33). Packed cell volume is a function of cell density and cell diameter; increases in packed cell volume could arise from increases in either cell density or cell diameter or both. Packed cell volume is a measure of the solid content in the cell culture. Solids are removed during harvest and downstream purification. More solids mean more effort to separate the solid material from the desired product during harvest and downstream purification steps. Also, the desired product can become trapped in the solids and lost during the harvest process, resulting in a decreased product yield. Since host cells vary in size and cell cultures also contain dead and dying cells and other cellular debris, packed cell volume is a more accurate way to describe the solid content within a cell culture than cell density or viable cell density. For example, a 2000 L culture having a cell density of 50×10⁶ cells/ml would have vastly different packed cell volumes depending on the size of the cells. In addition, some cells, when in a growth-arrested state, will increase in size, so the packed cell volume prior to growth-arrest and post growth-arrest will likely be different, due to increase in biomass as a result to cell size increase.

As used herein, “growth-arrest”, which may also be referred to as “cell growth-arrest”, is the point where cells stop increasing in number or when the cell cycle no longer progresses. Growth-arrest can be monitored by determining the viable cell density of a cell culture. Some cells in a growth-arrested state may increase in size but not number, so the packed cell volume of a growth-arrested culture may increase. Growth-arrest can be reversed to some extent, if the cells are not in declining health, by reversing the conditions that lead to growth arrest.

As used herein, the term “titer” means the total amount of a polypeptide or protein of interest (which may be a naturally occurring or recombinant protein of interest) produced by a cell culture in a given amount of medium volume. Titer can be expressed in units of milligrams or micrograms of polypeptide or protein per milliliter (or other measure of volume) of medium.

As used herein, the term “fed-batch culture” refers to a form of suspension culture and means a method of culturing cells in which additional components are provided to the culture at a time or times subsequent to the beginning of the culture process. The provided components typically comprise nutritional supplements for the cells which have been depleted during the culturing process. Additionally or alternatively, the additional components may include supplementary components (e.g., a cell cycle inhibitory compound). A fed-batch culture is typically stopped at some point and the cells and/or components in the medium are harvested and optionally purified.

As used herein, the terms “integrated viable cell density” or “IVCD” are used interchangeably and mean the average density of viable cells over the course of the culture multiplied by the amount of time the culture has run.

The term “alkyl” refers to a saturated, branched or straight-chain monovalent hydrocarbon group derived by the removal of one hydrogen atom from a single carbon atom of a parent alkane. Typical alkyl groups include, but are not limited to, methyl (—CH₃); ethyl (—CH₂CH₃); propyls such as propan-1-yl (—CH₂CH₂CH₃), and propan-2-yl (—CH(CH₂)₂); and butyls such as butan-1-yl (—CH₂CH₂CH₂CH₃), butan-2-yl, —CH(CH₃)CH₂CH₃ 2-methyl-propan-1-yl (—CH₂CH(CH₃)₂, 2-methyl-propan-2-yl (—C(CH₃)₃), and tert-butyl (—C(CH₃)₃); and the like. In certain embodiments, an alkyl group comprises 1 to 20 carbon atoms. In some embodiments, alkyl groups include 1 to 6 carbon atoms whereas in other embodiments, alkyl groups include 1 to 4 or 1 to 3 carbon atoms. In still other embodiments, an alkyl group includes 1 or 2 carbon atoms. Branched chain alkyl groups include at least 3 carbon atoms and typically include 3 to 7, or in some embodiments, 3 to 6 carbon atoms. An alkyl group having 1 to 6 carbon atoms may be referred to as a (C₁-C₆)alkyl or (C₁-C₆) alkyl group, an alkyl group having 1 to 4 carbon atoms may be referred to as a —(C₁-C₄)alkyl or (C₁-C₄) alkyl, and an alkyl group having 1 to 3 carbon atoms may be referred to as a (C₁-C₃)alkyl or —(C₁-C₃) alkyl. The same designation system applies to alkyl groups with different numbers of carbon atoms. Alkyl groups may be substituted or may be unsubstituted. In some embodiments, alkyl groups are unsubstituted. In other embodiments, an alkyl group may be substituted with one or more substituents. For example, in some embodiments, an alkyl group may be substituted with 1, 2 or 3 substituents whereas in another embodiment, an alkyl group may, where permitted by valence, be substituted with 1 to 5 substituents.

The term “alkoxy” refers to a radical OR where R represents a straight or branched chain alkyl group as defined above. Representative examples include, but are not limited to, methoxy (—OCH₃), ethoxy (—OCH₂CH₃), propoxy (—OCH₂CH₂CH₃), isopropoxy (—OCH(CH₃)₂), butoxy (—OCH₂CH₂CH₂CH₃), pentoxy(-OCH₂CH₂CH₂CH₂CH₃), t-butoxy (—OC(CH₃)₃) and the like. Typical alkoxy groups include 1 to 10 carbon atoms, 1 to 6 carbon atoms, 1 to 4 carbon atoms, 1 to 3 carbon atoms, or 1 to 2 carbon atoms in the R group. Alkoxy groups that include 1 to 6 carbon atoms may be designated as —O—(C₁-C₆ alkyl) groups. Similarly, alkoxy groups that include 1 to 3 carbon atoms may be designated as O—(C₁-C₃ alkyl) groups. Other alkoxy groups may be represented using the same methodology.

The term “carboxy” refers to the radical —C(O)OH which may alternatively be written as —C(═O)OH, —C(═O)—OH, COOH or —CO₂H. When the H atom of a carboxy group is removed and replaced with a bond to an alkyl group, the group may be written as —C(═O)—O— alkyl. Typical such groups include alkyl groups with 1 to 10 carbon atoms, 1 to 6 carbon atoms, 1 to 4 carbon atoms, 1 to 3 carbon atoms, or 1 to 2 carbon atoms. —C(═O)—O— alkyl groups that include alkyl groups with 1 to 6 carbon atoms may be designated as —C(═O)—O—(C₁-C₆ alkyl) groups. Similarly, such groups that include alkyl groups with 1 to 4 or 1 to 3 carbon atoms may be respectively designated as —C(═O)—O—(C₁-C₄ alkyl) and —C(═O)—O—(C₁-C₃ alkyl) groups. Other such groups may be represented using the same methodology.

The term “carbonyl” refers to a radical —C(═O)—. Carbonyl groups may be bonded to alkyl groups and written as —C(═O)-alkyl groups where alkyl has the meaning set forth above. Typical alkyl groups in such —C(═O)-alkyl groups have 1 to 10 carbon atoms, 1 to 6 carbon atoms, 1 to 4 carbon atoms, 1 to 3 carbon atoms, or 1 to 2 carbon atoms. The —C(═O)-alkyl groups with alkyl groups of 1 to 6 carbon atoms may be designated as —C(═O)—(C₁-C₆ alkyl) groups. Similarly, such groups where the alkyl groups have 1 to 4 or 1 to 3 carbon atoms may be respectively designated as —C(═O)—(C₁-C₄ alkyl) and —C(═O)—(C₁-C₃ alkyl) groups. Other such groups may be represented using the same methodology.

The term “cyano” refers to the radical CN which may also be written as —C≡N.

The term “cycloalkyl” refers to a saturated cyclic alkyl group derived by the removal of one hydrogen atom from a single carbon atom of a parent cycloalkane. Typical cycloalkyl groups include, but are not limited to, cyclopropane, cyclobutane, cyclopentane, cyclohexane, cycloheptane and the like. In certain embodiments, the cycloalkyl group can be C₃-C₁₀ cycloalkyl, such as, for example, C₃-C₆ cycloalkyl. In some embodiments, a cycloalkyl group is a C₅-C₇ group such as a cyclopentyl, cyclohexyl, or cycloheptyl group. Cycloalkyl groups may be substituted or unsubstituted.

The term “aryl”, alone or in combination, means a carbocyclic aromatic system containing one or two rings wherein such rings may be attached together in a fused manner. The term “aryl” embraces aromatic radicals such as phenyl, naphthyl, indenyl, tetrahydronaphthyl, and indanyl. More preferred aryl is phenyl. Said “aryl” group may have 1 to 3 substituents such as lower alkyl, hydroxyl, halo, haloalkyl, nitro, cyano, alkoxy and lower alkylamino.

The term “heterocyclyl group” refers to a cycloalkyl group, except that in a heterocyclyl group at least one ring atom is replaced by a heteroatom. Typically, heterocyclyl groups are characterized by the number of ring members and include 1, 2, or 3 heteroatoms independently selected from N, O, or S. In some embodiments, the heterocyclyl group can have 3 to 10 ring members, from 3 to 7 ring members, or from 5 to 7 ring members. In some embodiments, a heterocyclyl group is a 5 to 7 membered ring that includes 1, 2, or 3 heteroatoms independently selected from N, O, or S. Examples of heterocyclyl groups include, but are not limited to, aziridinyl, azetidinyl, oxetanyl, thiatanyl, tetrahydrofuranyl, tetrahydrothiophenyl, pyrrolidinyl, piperazinyl, piperidinyl, tetrahydro-2H-pyranyl, tetrahydro-2H-thiopyranyl, morpholinyl, thiomorpholinyl, azepanyl, oxepanyl, thiepanyl, and the like. Heterocyclyl groups may be substituted or unsubstituted. Some examples of alkyl substituted heterocycles include N-methylmorpholinyl, N-methylpiperidinyl, N-ethylpiperidinyl, N-methylpiperazinyl, N-propylpiperazinyl, 3-methylpiperidinyl, 2-methylpiperidinyl, and the like.

The term “heteroaryl group” refers to an aryl group, except that in a heteroarl group at least one ring atom is replaced by a heteroatom. Examples of heteroaryl radicals, include unsaturated 5 to 6 membered heteromonocyclyl group containing 1 to 4 nitrogen atoms, for example, pyrrolyl, imidazolyl, pyrazolyl, 2-pyridyl, 3-pyridyl, 4-pyridyl, pyrimidyl, pyrazinyl, pyridazinyl, triazolyl [e.g., 4H-1,2,4-triazolyl, 1H-1,2,3-triazolyl, 2H-1,2,3-triazolyl]; unsaturated 5- to 6-membered heteromonocyclic group containing an oxygen atom, for example, pyranyl, 2-furyl, 3-furyl, etc.; unsaturated 5 to 6-membered heteromonocyclic group containing a sulfur atom, for example, 2-thienyl, 3-thienyl, etc.; unsaturated 5- to 6-membered heteromonocyclic group containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl]; unsaturated 5 to 6-membered heteromonocyclic group containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl]. The term also embraces radicals where heterocyclic radicals are fused/condensed with aryl radicals: unsaturated condensed heterocyclic group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl [e.g., tetrazolo [1,5-b]pyridazinyl]; unsaturated condensed heterocyclic group containing 1 to 2 oxygen atoms and 1 to 3 nitrogen atoms [e.g. benzoxazolyl, benzoxadiazolyl]; unsaturated condensed heterocyclic group containing 1 to 2 sulfur atoms and 1 to 3 nitrogen atoms [e.g., benzothiazolyl, benzothiadiazolyl]; and saturated, partially unsaturated and unsaturated condensed heterocyclic group containing 1 to 2 oxygen or sulfur atoms [e.g. benzofuryl, benzothienyl, 2,3-dihydro-benzo[1,4]dioxinyl and dihydrobenzofuryl]. Preferred heteroaryl radicals include quinolyl, isoquinolyl, imidazolyl, pyridyl, thienyl, thiazolyl, oxazolyl, furyl, and pyrazinyl. Other preferred heteroaryl radicals are 5- or 6-membered heteroaryl, containing one or two heteroatoms selected from sulfur, nitrogen and oxygen, selected from thienyl, furyl, pyrrolyl, indazolyl, pyrazolyl, oxazolyl, triazolyl, imidazolyl, pyrazolyl, isoxazolyl, isothiazolyl, pyridyl, piperidinyl and pyrazinyl.

The term “halo” or “halogen” refers to a fluoro (—F), chloro (—Cl), bromo (—Br), or iodo (—I) group.

The term “haloalkyl” refers to an alkyl group as defined above in which at least one hydrogen atom is replaced with a halogen. Thus, the term “haloalkyl” includes “monohaloalkyl” (an alkyl substituted with one halogen atom), “dihaloalkyl” (an alkyl substituted with two halogen atoms which may be the same or different), and “trihaloalkyl” (an alkyl substituted with three halogen atoms which may be the same or different). The term “polyhaloalkyl” refers to an alkyl group that is substituted with two or more halogen atoms. The term “perhaloalkyl” means, unless otherwise stated, an alkyl group in which each of the hydrogen atoms is replaced with a halogen atom. For example, the term “perhaloalkyl”, includes, but is not limited to, trifluoromethyl (—CF₃), pentachloroethyl, 1,1,1-trifluoro-2-bromo-2-chloroethyl, and the like.

The term “hydroxy” refers to the hydroxyl group (—OH).

The term “nitro” refers to a radical of formula NO₂.

The term “sulfonyl” refers to a radical S(═O)₂—, or alternatively —SO₂—. Sulfonyl groups are typically bonded to R groups and may be written as S(═O)₂—R or as —SO₂—R where R is a substituted or unsubstituted alkyl, cycloalkyl, or other specified group as defined herein. Representative examples where R is a straight chain alkyl, an alkylsulfonyl, include, but are not limited to, methylsulfonyl (—S(═O)₂—CH₃), ethylsulfonyl (—S(═O)₂—CH₂CH₃), propylsulfonyl (—S(═O)₂—CH₂CH₂CH₃), butylsulfonyl(-S(═O)₂—CH₂CH₂CH₂CH₃), and the like. Typical alkylsulfonyl groups include 1 to 10 carbon atoms, 1 to 6 carbon atoms, 1 to 4 carbon atoms, 1 to 3 carbon atoms, or 1 to 2 carbon atoms in the alkyl R group. Alkylsulfonyl groups that include 1 to 6 carbon atoms may be designated as —S(═O)₂—(C₁-C₆ alkyl) groups. Similarly, alkylsulfonyl groups that include 1 to 3 carbon atoms may be designated as —S(═O)₂—(C₁-C₃ alkyl) groups. Other alkylsulfonyl groups may be described using the same methodology.

The term “amino” refers to a radical —NR′R″ where R′ and R″ are independently chosen from —H, or substituted or unsubstituted straight or branched chain alkyl, cycloalkyl, or other specified group as defined herein. When R′ and R″ are both H, the NR′R″ group is a NH₂ group. When one of R′ and R″ is —H and the other is an alkyl group, the —NR′R″ is a —NH-alkyl which may also be designated as a —NH(alkyl) or —N(H)(alkyl) group. When R′ and R″ are both alkyl groups, the alkyl groups may be different, and the group may be designated as a N(alkyl)(alkyl) group. If R′ and R″ are both alkyl groups and the alkyl groups are the same, the group may be referred to as a N(alkyl)₂. The alkyl groups of the R′ and R″ may be designated based on the number of carbon atoms in the alkyl group. For example, an R′ or R″ alkyl group with 1 to 6 carbon atoms may be designated as a —(C₁-C₆ alkyl). Similarly, an R′ or R″ alkyl group with 1 to 4 carbon atoms may be designated as a —(C₁-C₄ alkyl). By way of nonlimiting example, a —NR′R″ group in which one of R′ and R″ is a —H and the other is an alkyl groups with 1-4 carbon atoms, may be referred to as a —NH(C₁-C₄ alkyl) group or as a —N(H)(C₁-C₄ alkyl) group. Similar methodology may be used to describe different —NR′R″ groups. Typical R′ and R″ alkyl groups of —NR′R″ groups include 1 to 10 carbon atoms, 1 to 6 carbon atoms, 1 to 4 carbon atoms, 1 to 3 carbon atoms, or 1 to 2 carbon atoms.

The term “carboxamide” as used herein, refers to a group of formula —C(═O)—NR′R″ which may also be referred to a —C(═O)NR′R″ where R′ and R″ are independently chosen from —H, or substituted or unsubstituted straight or branched chain alkyl, cycloalkyl, or other specified group as defined herein. When R′ and R″ are both —H, the carboxamide may be written as a —C(═O)NH₂ or —C(═O)—NH₂ group. When one of R′ and R″ is —H and the other is an alkyl group, the carboxamide is a —C(═O)—NH— alkyl which may also be designated as a —C(═O)—N(H)-alkyl, —C(═O)—N(H)(alkyl), —C(═O)N(H)-alkyl, or —C(═O)N(H)(alkyl) group. When R′ and R″ are both alkyl groups, the alkyl groups may be different, and the group may be designated as a —C(═O)—N(alkyl)(alkyl) group or as a —C(═O)N(alkyl)(alkyl) group. If R′ and R″ are both alkyl groups and the alkyl groups are the same, the group may be referred to as a —C(═O)—N(alkyl)₂, or as a —C(═O)N(alkyl)₂ group. The alkyl groups of the R′ and R″ groups may be designated based on the number of carbon atoms in the alkyl group. For example, an R′ or R″ alkyl group with 1 to 6 carbon atoms may be designated as a —(C₁-C₆ alkyl). Similarly, an R′ or R″ alkyl group with 1 to 4 carbon atoms may be designated as a —(C₁-C₄ alkyl). By way of nonlimiting example, a —C(═O)—NR′R″ group in which one of R′ and R″ is a —H and the other is an alkyl groups with 1-4 carbon atoms, may be referred to as a —C(═O)—NH(C₁-C₄ alkyl) group or as a —C(═O)—N(H)(C₁-C₄ alkyl) group. Similar methodology may be used to describe different —C(═O)—NR′R″ groups. Typical R′ and R″ alkyl groups include 1 to 10 carbon atoms, 1 to 6 carbon atoms, 1 to 4 carbon atoms, 1 to 3 carbon atoms, or 1 to 2 carbon atoms.

The term “sulfonamide” as used herein, refers to a group of formula —S(═O)₂—NR′R″ which may also be referred to a —S(═O)₂NR′R″ where R′ and R″ are independently chosen from —H, or substituted or unsubstituted straight or branched chain alkyl, cycloalkyl, or other specified group as defined herein. When R′ and R″ are both —H, the sulfonamide may be written as a —S(═O)₂NH₂ or —S(═O)₂—NH₂ group. When one of R′ and R″ is —H and the other is an alkyl group, the sulfonamide is a —S(═O)₂—NH-alkyl which may also be designated as a —S(═O)₂—N(H)-alkyl, —S(═O)₂—N(H)(alkyl), —S(═O)₂N(H)-alkyl, or —S(═O)₂N(H)(alkyl) group. When R′ and R″ are both alkyl groups, the alkyl groups may be different, and the group may be designated as a —S(═O)₂—N(alkyl)(alkyl) group or as a —S(═O)₂N(alkyl)(alkyl) group. If R′ and R″ are both alkyl groups and the alkyl groups are the same, the group may be referred to as a —S(═O)₂—N(alkyl)₂, or as a —S(═O)₂N(alkyl)₂ group. The alkyl groups of the R′ and R″ may be designated based on the number of carbon atoms in the alkyl group. For example, an R′ or R″ alkyl group with 1 to 6 carbon atoms may be designated as a —(C₁-C₆ alkyl). Similarly, an R′ or R″ alkyl group with 1 to 4 carbon atoms may be designated as a —(C₁-C₄ alkyl). By way of nonlimiting example, a —S(═O)₂—NR′R″ group in which one of R′ and R″ is a —H and the other is an alkyl groups with 1-4 carbon atoms, may be referred to as a —S(═O)₂—NH(C₁-C₄ alkyl) group or as a —S(═O)₂—N(H)(C₁-C₄ alkyl) group. Similar methodology may be used to describe different —S(═O)₂—NR′R″ groups. Typical R′ and R″ alkyl groups include 1 to 10 carbon atoms, 1 to 6 carbon atoms, 1 to 4 carbon atoms, 1 to 3 carbon atoms, or 1 to 2 carbon atoms.

### Description of Cell Culture Process

During recombinant protein production it is desirable to have a controlled system where cells are grown to a desired density and then the physiological state of the cells is switched to a growth-arrested, high productivity state where the cells use energy and substrates to produce the recombinant protein of interest instead of making more cells. Methods for accomplishing this goal, such as temperature shifts and amino acid starvation, are not always successful and can have undesirable effects on product quality. As described herein, packed cell volume can be limited to a desired level during the production phase by inducing cell growth-arrest in the cultured cells by exposure to cell cycle inhibitors. Cell growth-arrest can be achieved and maintained by using a cell culture medium that contains a cell cycle inhibitor.

It was also found that the growth-arrested cells showed increased productivity when growth-arrest was initiated by contact with cell cycle inhibitors and the growth-arrested cells were subsequently maintained with the cell culture and cell culture medium comprising a cell cycle inhibitor.

A growth-arrested, high productivity production phase can be achieved by manipulating the concentration of cell cycle inhibitor. As described herein, inclusion of cell cycle inhibitors in cell culture medium resulted in growth-arrest.

Cell cycle inhibitors include compounds targeting various pathways involved in the cell cycle, including transcription, co-transcription modification, translation, co-translation modification, and secretion of proteins, as well as glycosylation thereof. Desirable characteristics of cell cycle inhibitors include the ability to induce growth arrest without inducing cell death, maintenance of normal transcriptional and translational functions in the growth-arrested cells, and a sufficient degree of water solubility to facilitate incorporation of the cell cycle inhibitor into culture medium.

Representative steps in the cell cycle were identified as points at which intervention may modulate the cell cycle and induce growth arrest. Points of intervention included modulation of cyclin-dependent kinases (CDKs) and their substrates, as well as endogenous proteins that affect these cell cycle regulators. In particular, CDK inhibitors were found to inhibit proliferation in a dose-dependent manner, mediate a G1 arrest without inducing cell death and increase specific productivity of recombinant protein. Thus, the cell mass was controlled without sacrificing the accumulated titer. The compounds include those described in more detail herein.

For commercial scale cell culture and the manufacture of biological therapeutics, the ability to arrest cell growth and maintain the cells in a growth-arrested state during the production phase would be very desirable, enabling the cells' biosynthetic capacity to be focused upon product synthesis. Having cells that were also induced to increase specific productivity (product per cell) while in the growth-arrested state and being able to maintain this increased productivity, is ideal for manufacturing purposes.

Provided herein is a method of arresting cell growth in a mammalian cell culture expressing a recombinant protein. The method includes inducing cell growth-arrest in a mammalian cell culture by subjecting the cell culture to culture medium comprising a cell cycle inhibitor. Such induction can be initiated by adding a cell cycle inhibitor into the culture medium during production, for example, during batch culture, or perfusing the culture with a culture medium comprising a cell cycle inhibitor, or via the addition of the inhibitor via a feed or supplement into the process, as in a typical fed batch bioreactor. The cell culture is then maintained in the growth-arrested state by continued exposure to culture medium with a cell cycle inhibitor (for example, by inclusion of inhibitor in culture medium used in a perfusion process), or regular additions of the inhibitor at specific time points to continue the arrest.

Also provided is a method for increasing recombinant protein production in a mammalian cell culture expressing a recombinant protein by inducing cell growth-arrest via focusing the cells activities towards therapeutic protein production at the expense of cell growth. Mammalian cells maintained in the growth-arrested state exhibited greater specific productivity (g protein/cell/day and g protein/cell mass/day) than those which were not growth-arrested.

Such a method is also useful for limiting a mammalian cell culture at a desired packed cell volume. Packed cell volume during the production phase could be limited at a desired level by introducing a cell cycle inhibitor in the production culture medium. This enables greater control over the process, and represents an additional level of process control, enabling more precise parameters to be utilized. Various cell cycle inhibitors, which can be used at selected concentrations, are disclosed herein.

The methods described herein provide greater control over cell growth to increase product titer in cell culture processes; and as such can simplify the gassing strategy compared to a high biomass perfusion processes and minimize product loss during harvest and downstream processing.

The method begins with establishing a mammalian cell production culture in a culture plate, flask, tube, or bioreactor. Smaller production bioreactors are typically used, in one embodiment the bioreactors are 500 L to 2000 L. In another embodiment, 1000 L-2000 L bioreactors are used. The seed cell density used to inoculate the bioreactor can have a positive impact on the level of recombinant protein produced. In one embodiment the bioreactor is inoculated with at least 0.5×10⁶ up to and beyond 3.0×10⁶ viable cells/mL in a culture medium. In another embodiment the inoculation is 1.0×10⁶ viable cells/mL.

The mammalian cells then undergo an exponential growth phase. The cell culture can be maintained with or without supplemental feeding until a desired cell density is achieved. In one embodiment the cell culture is maintained for up to three days with or without supplemental feeding followed by adding a cell cycle inhibitor to induce growth-arrest. The culture can be maintained in the presence of the cell cycle inhibitor, to keep the cells in a state of growth arrest. In another embodiment the culture can be inoculated at a desired cell density to begin the production phase without a brief growth phase, with cell growth-arrest initiated immediately upon inoculation by contacting the cell culture with culture medium containing a cell cycle inhibitor to induce and maintain growth-arrest. In any of the embodiments herein the switch from the growth phase to production phase can also be initiated by contacting the cells with a cell cycle inhibitor followed by adding cell cycle inhibitors to the medium, or perfusion with a cell culture medium comprising a cell cycle inhibitor, and maintaining the cell culture in the presence of the cell cycle inhibitor. In any of the embodiments, additional cell cycle inhibitor can be added to the medium at an interval sufficient to maintain a desired concentration of cell cycle inhibitor.

Without regard as to how growth-arrest is induced, higher productivity is seen in the growth-arrested cells that are maintained by adding a cell cycle inhibitor and maintaining the cell culture in the medium comprising a cell cycle inhibitor.

At the transition between the growth phase and the production phase, and during the production phase, the percent packed cell volume (% PCV) is equal to or less than 35%. The desired packed cell volume maintained during the production phase is equal to or less than 35%. In one embodiment the packed cell volume is equal to or less than 30%. In another embodiment the packed cell volume is equal to or less than 20%. In yet another embodiment the packed cell volume is equal to or less than 15%. In a further embodiment the packed cell volume is equal to or less than 10%.

The desired viable cell density at the transition between the growth and production phases and maintained during the production phase can be varied depending on the nature of the cells and/or the recombinant protein being expressed. It can be decided based on the equivalent packed cell volume from historical data developed with a particular cell line. In one embodiment, the viable cell density is at least about 10×10⁶ viable cells/mL to 80×10⁶ viable cells/mL. In one embodiment the viable cell density is at least about 10×10⁶ viable cells/mL to 70×10⁶ viable cells/mL. In one embodiment the viable cell density is at least about 10×10⁶ viable cells/mL to 60×10⁶ viable cells/mL. In one embodiment the viable cell density is at least about 10×10⁶ viable cells/mL to 50×10⁶ viable cells/mL. In one embodiment the viable cell density is at least about 10×10⁶ viable cells/mL to 40×10⁶ viable cells/mL. In another embodiment the viable cell density is at least about 10×10⁶ viable cells/mL to 30×10⁶ viable cells/mL. In another embodiment the viable cell density is at least about 10×10⁶ viable cells/mL to 20×10⁶ viable cells/mL. In another embodiment, the viable cell density is at least about 20×10⁶ viable cells/mL to 30×10⁶ viable cells/mL. In another embodiment the viable cell density is at least about 20×10⁶ viable cells/mL to at least about 25×10⁶ viable cells/mL, or at least about 20×10⁶ viable cells/mL.

Lower packed cell volume during the production phase helps mitigate dissolved oxygen sparging problems that can hinder higher cell density perfusion cultures and lead to potential control and cell viability problems. The lower packed cell volume also allows for a smaller media volume to be utilized, which in turn allows for the use of smaller media storage vessels and can be combined with slower media flow rates. Lower packed cell volume also has less impact on harvest and downstream processing, compared to higher cell biomass cultures, where high packed cell volumes may represent a problem at the harvest step leading to lost protein product. All of these factors may reduce the costs associated with manufacturing recombinant protein therapeutics.

Three methods are typically used in commercial processes for the production of recombinant proteins by mammalian cell culture: batch culture, fed-batch culture, and perfusion culture. Batch culture is a discontinuous method where cells are grown in a fixed volume of culture media for a short period of time followed by a full harvest. Cultures grown using the batch method experience an increase in cell density until a maximum cell density is reached, followed by a decline in viable cell density as the media components are consumed and levels of metabolic by-products (such as lactate and ammonia) accumulate. Harvest typically occurs at or soon after the point when the maximum cell density is achieved (typically 5-10×10⁶ cells/mL, depending on media formulation, cell line, etc). The batch process is the simplest culture method, however viable cell density is limited by the nutrient availability and once the cells are at maximum density, the culture declines and production decreases. There is no ability to extend a production phase because the accumulation of waste products and nutrient depletion rapidly lead to culture decline, (typically around 3 to 7 days).

Fed-batch culture improves on the batch process by providing bolus or continuous media feeds to replenish those media components that have been consumed. Since fed-batch cultures receive additional nutrients throughout the culture process, they have the potential to achieve higher cell densities (>10 to 30×10⁶ cells/ml, depending on media formulation, cell line, etc.) and increased product titers, when compared to the batch method. Unlike the batch process, a biphasic culture can be created and sustained by manipulating feeding strategies and media formulations to distinguish the period of cell proliferation to achieve a desired cell density (the growth phase) from the period of suspended or slow cell growth (the production phase). As such, fed batch cultures have the potential to achieve higher product titers compared to batch cultures. Typically a batch method is used during the growth phase and a fed-batch method used during the production phase, but a fed-batch feeding strategy can be used throughout the entire process. However, unlike the batch process, bioreactor volume is a limiting factor which limits the amount of feed that can be provided during the fed-batch process. Also, as with the batch method, metabolic by-product accumulation will lead to declining cell viability over time as these progressively accumulate within the cell culture media, which limits the duration of the production phase, about 1.5 to 3 weeks. Fed-batch cultures are discontinuous and harvest typically occurs when metabolic by-product levels or the culture viability reach predetermined levels. When compared to a batch culture, in which no feeding occurs, a fed batch culture can produce significantly higher amounts of recombinant protein. See e.g. U.S. Pat. No. 5,672,502.

Perfusion based methods offer potential improvement over the batch and fed-batch methods by adding fresh media and simultaneously removing spent media. Typical large scale commercial cell culture strategies strive to reach high cell densities, 60-90(+)×10⁶ cells/mL, at which point (depending on cell size) almost a third to over one-half of the reactor volume is biomass. With perfusion based culture, extreme cell densities of >1×10⁸ cells/mL have been achieved and even higher densities are predicted. Typical perfusion cultures begin with a batch culture start-up lasting for a day or more to enable rapid initial cell growth and biomass accumulation, followed by continuous, step-wise and/or intermittent addition of fresh feed media to the culture and simultaneous removal of spent media with the retention of cells and additional high molecular weight compounds such as proteins (based on the filter molecular weight cutoff) throughout the growth and production phases of the culture. Various methods, such as sedimentation, centrifugation, or filtration, can be used to remove spent media, while maintaining cell density. Perfusion flow rates of a fraction of a working volume per day up to many multiple working volumes per day have been utilized.

An advantage of the perfusion process is that the production culture can be maintained for longer periods than batch or fed-batch culture methods, due to removal of metabolic waste products and the supplementation with fresh media/feeds being balanced by removal of bioreactor volume, enabling a steady state level to be achieved. However, increased media preparation, use, storage and disposal are necessary to support a long term perfusion culture, particularly those with high cell densities, which also need even more nutrients, and all of this drives the production costs even higher, compared to batch and fed batch methods. In addition, higher cell densities can cause problems during production, such as maintaining dissolved oxygen levels and problems with increased gassing including supplying more oxygen and removing more carbon dioxide, which would result in more foaming and the need for alterations to antifoam strategies; as well as during harvest and downstream processing where the efforts required to remove the excessive cell material can result in loss of product, negating the benefit of increased titer due to increased cell mass.

Also provided is a large scale cell culture strategy that combines fed batch feeding during the growth phase followed by continuous perfusion during the production phase. The method targets a production phase where the cell culture is maintained at a packed cell volume of less than or equal to 35%. The method also provides the initiation and maintenance of cell growth-arrest due to use of a medium comprising a cell cycle inhibitor.

In one embodiment, a fed-batch culture with bolus feeds is used to maintain a cell culture during the growth phase targeting rapid cell proliferation and biomass accumulation. Perfusion based feeding strategies can then be used during a production phase. In one embodiment, perfusion begins when the cells have reached a biomass range suitable for the production phase. In another embodiment, perfusion begins on or about day 5 to on or about day 9 of the cell culture. In another embodiment perfusion begins on or about day 5 to on or about day 7 of the cell culture.

In another embodiment the initiation of cell growth-arrest in the fed-batch culture can be initiated by exposing the fed-batch culture to a cell cycle inhibitor. In one embodiment the cell cycle inhibitor concentration of the cell culture medium is monitored. In another embodiment the initiation of cell growth-arrest in the fed-batch culture can be achieved by addition of a culture medium comprising a cell cycle inhibitor. In further embodiments, additional cell cycle inhibitor(s) can be added at selected time points during the culture process.

Using bolus feeding during the growth phase allows the cells to transition into the production phase, resulting in less dependence on a temperature shift as a means of initiating and controlling the production phase, however a temperature shift of 36° C. to 31° C. can take place between the growth phase and production phase. In one embodiment the shift is from 36° C. to 33° C.

As described herein, the bioreactor can be inoculated with at least 0.5×10⁶ up to and beyond 3.0×10⁶ viable cells/mL in a culture medium, for example 1.0×10⁶ viable cells/mL.

Perfusion culture is one in which the cell culture receives fresh culture medium while simultaneously removing spent medium. Perfusion can be continuous, step-wise, intermittent, or a combination of any or all of any of these. Perfusion rates can be less than a working volume to many working volumes per day. The cells are retained in the culture and the spent medium that is removed is substantially free of cells or has significantly fewer cells than the culture. Recombinant proteins expressed by the cell culture can also be retained in the culture. Perfusion can be accomplished by a number of means including centrifugation, sedimentation, or filtration, See e.g. Voisard et al., (2003), Biotechnology and Bioengineering 82:751-65. An example of a filtration method is alternating tangential flow filtration. Alternating tangential flow is maintained by pumping medium through hollow-fiber filter modules. See e.g. U.S. Pat. No. 6,544,424; Furey (2002) Gen. Eng. News. 22 (7), 62-63.

As used herein, “perfusion flow rate” is the amount of media that is passed through (added and removed) from a bioreactor, typically expressed as some portion or multiple of the working volume, in a given time. “Working volume” refers to the amount of bioreactor volume used for cell culture. In one embodiment the perfusion flow rate is one working volume or less per day. Culture medium (for example, a perfusion feed medium) can be formulated to maximize perfusion nutrient concentration to minimize perfusion rate.

Cell cultures can be supplemented with concentrated medium containing components, such as nutrients and amino acids, which are consumed during the course of the production phase of the cell culture. Concentrated medium may be based on just about any cell culture media formulation. Such a concentrated medium can contain most of the components of the cell culture medium at, for example, about 5×, 6×, 7×, 8×, 9×, 10×, 12×, 14×, 16×, 20×, 30×, 50×, 100×, 200×, 400×, 600×, 800×, or even about 1000× of their normal amount. Concentrated feed media are often used in fed batch culture processes.

The method according to the present invention may be used to improve the production of recombinant proteins in multiple phase culture processes. In a multiple stage process, cells are cultured in two or more distinct phases. For example cells may be cultured first in one or more growth phases, under environmental conditions that maximize cell proliferation and viability, then transferred to a production phase, under conditions that maximize protein production. In a commercial process for production of a protein by mammalian cells, there are commonly multiple, for example, at least about 2, 3, 4, 5, 6, 7, 8, 9, or 10 growth phases that occur in different culture vessels preceding a final production culture.

The growth and production phases may be preceded by, or separated by, one or more transition phases. In multiple phase processes, the method according to the present invention can be employed at least during the growth and production phase of the final production phase of a commercial cell culture, although it may also be employed in a preceding growth phase. A production phase can be conducted at large scale. A large scale process can be conducted in a volume of at least about 100, 500, 1000, 2000, 3000, 5000, 7000, 8000, 10,000, 15,000, 20,000 liters. In one embodiment production is conducted in 500 L, 1000 L and/or 2000 L bioreactors.

A growth phase may occur at a higher temperature than a production phase. For example, a growth phase may occur at a first temperature from about 35° C. to about 38° C., and a production phase may occur at a second temperature from about 29° C. to about 37° C., optionally from about 30° C. to about 36° C. or from about 30° C. to about 34° C. In addition, chemical inducers of protein production, such as, for example, caffeine, butyrate, and hexamethylene bisacetamide (HMBA), may be added at the same time as, before, and/or after a temperature shift. If inducers are added after a temperature shift, they can be added from one hour to five days after the temperature shift, optionally from one to two days after the temperature shift. The cell cultures can be maintained for days or even weeks while the cells produce the desired protein(s).

One or more small molecule inhibitors of cell growth could be added to the culture in a similar manner. The timing of the addition of the inhibitor and/or the frequency of the use of the inhibitor can be driven by the performance/cellular growth rates of the process itself according to process monitoring, or via specific target timepoint(s) within the process. For example, the inhibitor could be added as part of a feed, or as a single supplement, once the cells reach a specific cell density or packed cell volume. Cell growth can be monitored post addition and additional supplementation with the inhibitor can be triggered upon a subsequent increase in cell growth post addition. Alternatively, once the initial inhibitor treatment has occurred, regular additions of the inhibitor could be timed at daily intervals or at specific timepoints, for example after every 2-4 days of production.

Cell cycle inhibitors, such as the ones that directly interact with cell cycle machinery, including CDKs, and cyclins, are useful in the present methods. Additional useful cell cycle inhibitors include molecules that interact with proteins from other pathways, such as AKT, mTOR, and other pathways that affect, directly or indirectly, the cell cycle.

Samples from the cell culture can be monitored and evaluated using any of the analytical techniques known in the art. A variety of parameters including recombinant protein and medium quality and characteristics can be monitored for the duration of the culture. Samples can be taken and monitored intermittently at a desirable frequency, including continuous monitoring, real time or near real time.

Typically the cell cultures that precede the final production culture (N-x to N-1) are used to generate the seed cells that will be used to inoculate the production bioreactor, the N-1 culture. The seed cell density can have a positive impact on the level of recombinant protein produced. Product levels tend to increase with increasing seed density. Improvement in titer is tied not only to higher seed density, but is likely to be influenced by the metabolic and cell cycle state of the cells that are placed into production.

Seed cells can be produced by any culture method. One such method is a perfusion culture using alternating tangential flow filtration. An N-1 bioreactor can be run using alternating tangential flow filtration to provide cells at high density to inoculate a production bioreactor. The N-1 stage may be used to grow cells to densities of >90×10⁶ cells/mL. The N-1 bioreactor can be used to generate bolus seed cultures or can be used as a rolling seed stock culture that could be maintained to seed multiple production bioreactors at high seed cell density. The duration of the growth stage of production can range from 7 to 14 days and can be designed so as to maintain cells in exponential growth prior to inoculation of the production bioreactor. Perfusion rates, medium formulation and timing are optimized to grow cells and deliver them to the production bioreactor in a state that is most conducive to optimizing their production. Seed cell densities of >15×10⁶ cells/mL can be achieved for seeding production bioreactors. Higher seed cell densities at inoculation can decrease or even eliminate the time needed to reach a desired production density.

The invention finds particular utility in improving cell growth, viability and/or protein production via cell culture processes. The cell lines (also referred to as “host cells”) used in the invention are genetically engineered to express a polypeptide of commercial or scientific interest. Cell lines are typically derived from a lineage arising from a primary culture that can be maintained in culture for an unlimited time. Genetically engineering the cell line involves transfecting, transforming or transducing the cells with a recombinant polynucleotide molecule, and/or otherwise altering (e.g., by homologous recombination and gene activation or fusion of a recombinant cell with a non-recombinant cell) so as to cause the host cell to express a desired recombinant polypeptide. Methods and vectors for genetically engineering cells and/or cell lines to express a polypeptide of interest are well known to those of skill in the art; for example, various techniques are illustrated in Current Protocols in Molecular Biology, Ausubel et al., eds. (Wiley & Sons, New York, 1988, and quarterly updates); Sambrook et al., Molecular Cloning: A Laboratory Manual (Cold Spring Laboratory Press, 1989); Kaufman, R. J., Large Scale Mammalian Cell Culture, 1990, pp. 15-69.

Animal cell lines are derived from cells whose progenitors were derived from a multicellular animal. One type of animal cell line is a mammalian cell line. A wide variety of mammalian cell lines suitable for growth in culture are available from the American Type Culture Collection (Manassas, Va.) and commercial vendors. Examples of cell lines commonly used in the industry include VERO, BHK, HeLa, CV1 (including Cos), MDCK, 293, 3T3, myeloma cell lines (e.g., NSO, NS1), PC12, W138 cells, and Chinese hamster ovary (CHO) cells. CHO cells are widely used for the production of complex recombinant proteins, e.g. cytokines, clotting factors, and antibodies (Brasel et al. (1996), Blood 88:2004-2012; Kaufman et al. (1988), J. Biol Chem 263:6352-6362; McKinnon et al. (1991), J Mol Endocrinol 6:231-239; Wood et al. (1990), J. Immunol. 145:3011-3016). The dihydrofolate reductase (DHFR)-deficient mutant cell lines (Urlaub et al. (1980), Proc Natl Acad Sci USA 77: 4216-4220), DXB11 and DG-44, are desirable CHO host cell lines because the efficient DHFR selectable and amplifiable gene expression system allows high level recombinant protein expression in these cells (Kaufman R. J. (1990), Meth Enzymol 185:537-566). In addition, these cells are easy to manipulate as adherent or suspension cultures and exhibit relatively good genetic stability. CHO cells and proteins recombinantly expressed in them have been extensively characterized and have been approved for use in clinical commercial manufacturing by regulatory agencies.

### Cell Cycle Inhibitors

As used herein, the term “cell cycle inhibitors” refers to any compound known or suspected to regulate cell cycle progression and the associated processes of transcription, DNA repair, differentiation, senescence and apoptosis related to this. Examples of cell cycle inhibitors include but are not limited to CDK inhibitors, for example, CDK4 inhibitors. Additional examples of cell cycle inhibitors are described herein and are known in the art.

Preferably, the cell cycle inhibitor has an IC₅₀ in a CDK4 enzyme inhibition assay similar to that described herein [Example 6] of less than about 20 nM. More preferably, the cell cycle inhibitor has an IC₅₀ in a CDK4 cell assay similar to that described herein [Example 7] of less than about 100 nM.

One embodiment of the invention of relates to cell cycle inhibitors of the Formula 1 or 2

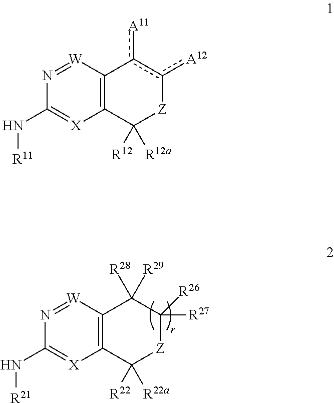

enantiomers, diastereomers, salts and solvates thereof wherein
A¹¹ and A¹² together with ring carbon atoms to which they are attached combine to form benzene, cyclopentadiene, pyridine, pyridone, pyrimidine, pyrazine, pyridazine, 2H-pyran, pyrrole, imidazole, pyrazole, triazole, furan, oxazole, isoxazole, oxadiazole, thiophene, thiazole, isothiazole or thiadiazole any of which may be optionally partially saturated, and any of which may be optionally independently substituted with one or more R^{x} groups as allowed by valance;
W and X are independently CH or N;
Z is absent, —O—, —S(O)ₙ—, or —NHR^{3b}—;
R¹¹ and R²¹ is —Y—R^{a} wherein
   Y is alkyl, alkenyl, alkynyl, cycloalkyl, cycloalkenyl, heterocyclyl, aryl or heteroaryl any of which may be optionally independently substituted with one or more R^{x} groups as allowed by valance; and
   R^{a} is heterocyclo, —NR^{3b}R^{4b}, —C(═O)NR^{3b}R^{4b}; —O—R^{5b}, or —S(O)ₙ—R^{5b};
R¹² and R^{12a} and R²² and R^{22a} are each independently alkyl or alkenyl either of which may be optionally substituted with one or more R^{x} as allowed by valance;
or R¹² and R^{12a} or R²² and R^{22a} together with the ring carbon atom to which they are attached combine to form a spiro-fused ring system of the following formula 1A

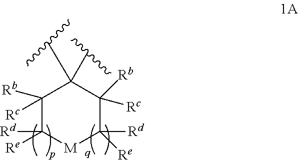

wherein
M is —CR^{d}R^{e}—, —O—, —S(O)ₙ—, or —NHR^{b}—;
R^{b}, R^{c}, R^{d} and R^{e} are each independently H or R^{x}, or alternatively R^{b} and R^{d} on adjacent carbon ring atoms may optionally combine to form a double bond as allowed by valance, and R^{d} and R^{e} on adjacent carbon ring atoms may optionally combine to form a double bond as allowed by valance;
R^{3b} and R^{4b} at each occurrence are independently
   (i) hydrogen or
   (ii) alkyl, cycloalkyl, heterocyclo, aryl, heteroaryl, cycloalkylalkyl, heterocycloalkyl, arylalkyl, or heteroarylalkyl any of which may be optionally independently substituted with one or more R^{x} groups as allowed by valance;
or R^{3b} and R^{4b} together with the nitrogen atom to which they are attached may combine to form a heterocyclo ring optionally independently substituted with one or more R^{x} groups as allowed by valance;
R^{3b}* and R^{4b}* at each occurrence are independently
   (i) hydrogen or
   (ii) alkyl, alkenyl, alkynyl cycloalkyl, heterocyclo, aryl, heteroaryl, cycloalkylalkyl, heterocycloalkyl, arylalkyl, or heteroarylalkyl any of which may be optionally independently substituted with one or more R^{x} groups as allowed by valance;
or R^{3b}* and R^{4b}* together with the nitrogen atom to which they are attached may combine to form a heterocyclo ring optionally independently substituted with one or more R^{x} groups as allowed by valance;
R^{5b} and R^{5b}* at each occurrence is
   (i) hydrogen or
   (ii) alkyl, alkenyl, alkynyl cycloalkyl, heterocyclo, aryl, heteroaryl, cycloalkylalkyl, heterocycloalkyl, arylalkyl, or heteroarylalkyl any of which may be optionally independently substituted with one or more R^{x} groups as allowed by valance;
R²⁶, R²⁷, R²⁸ and R²⁹ are each independently H or R^{x}, or alternatively
   (i) R²⁶ and R²⁷ together with the ring carbon atom to which they are attached may combine to form ═O, ═S or ═NR^{3b}, as allowed by valance;
   (ii) R²⁸ and R²⁹ together with the ring carbon atom to which they are attached may combine to form ═O, ═S or ═NR^{3b}, as allowed by valance;
   (iii) two R²⁶ groups on adjacent ring carbon atoms may combine to form a double bond as allowed by valance;
   (iv) R²⁶ and R²⁸ on adjacent ring carbon atoms may combine to form a double bond as allowed by valance;
R^{x} at each occurrence is independently, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, -(alkylene)ₘ-OR^{5b}, -(alkylene)ₘ-S(O)ₙR^{5b}, -(alkylene)ₘ-NR^{3b}R^{4b}, -(alkylene)ₘ-C(═O)R^{5b}, -(alkylene)ₘ-C(═S)R^{5b}, -(alkylene)ₘ-C(═O)OR^{5b}, -(alkylene)ₘ-OC(═O)R^{5b}, -(alkylene)ₘ-C(═S)OR^{5b}, -(alkylene)ₘ-C(═O)NR^{3b}R^{4b}, -(alkylene)ₘ-C(═S)NR^{3b}R^{4b}, -(alkylene)ₘ-N(R^{3b})C(═O)NR^{3b}R^{4b}, -(alkylene)ₘ-N(R^{3b})C(═S)NR^{3b}R^{4b}, -(alkylene)ₘN(R^{3b})C(═O)R^{5b}, -(alkylene)ₘ-N(R^{3b})C(═S)R^{5b}, -(alkylene)ₘ-OC(═O)NR^{3b}R^{4b}, -(alkylene)ₘ-OC(═S)NR^{3b}R^{4b}, -(alkylene)ₘ-SO₂NR^{3b}R^{4b}, -(alkylene)ₘ-N(R^{3b})SO₂R^{5b}, -(alkylene)ₘ-N(R^{3b})SO₂NR^{3b}R^{4b}, -(alkylene)ₘ-N(R^{3b})C(═O)OR^{5b}, -(alkylene)ₘ-N(R^{3b})C(═S)OR^{5b}, or -(alkylene)ₘ-N(R^{3b})SO₂R^{5b};
wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more -(alkylene)ₘ-OR^{5b}*, -(alkylene)ₘ-S(O)ₙR^{5b}*, -(alkylene)ₘ-NR^{3b}*R^{4b}*, -(alkyl ene)ₘ-C(═O)R^{5b}*, -(alkylene)ₘ-C(═S)R^{5b}*, -(alkylene)ₘ-C(═O)O R^{5b}*, -(alkylene)ₘ-OC(═O)R^{5b}*, -(alkylene)ₘ-C(═S)OR^{5b}* -(alkylene)ₘ-C(═O)NR^{3b}*R^{4b}*, -(alkylene)ₘ-C(═S)NR^{3b}*R^{4b}*, -(alkylene)ₘ-N(R^{3b}*)C(═O)NR^{3b}*R^{4b}*, -(alkylene)ₘ-N(R^{3b}*)C(═S)NR^{3b}*R^{4b}*, -(alkylene)ₘ-N(R^{3b}*)C(═O)R^{5b}*, -(alkylene)ₘ-N(R^{3b}*)C(═S)R^{5b}*, -(alkylene)ₘ-OC(═O)NR^{3b}*R^{4b}*, -(alkylen e)ₘ-OC(═S)NR^{3b}*R^{4b}*, -(alkylene)ₘ-SO₂NR^{3b}*R^{4b}*, -(alkylene)ₘ-N(R^{3b}*)SO₂R^{5b}*, -(alkylene)ₘ-N(R^{3b}*)SO₂NR^{3b}*R^{4b}*, -(alkylene)ₘ-N(R^{3b}*)C(═O)OR^{5b}*, -(alkylene)ₘ-N(R^{3b}*)C(═S)OR^{5b}*, or -(alkylene)ₘ-N(R^{3b}*)SO₂R^{5b}*;
n is 0, 1 or 2;
m is 0 or 1;
p and q are independently 0, 1 or 2; and
r is 1, 2 or 3 when Z is absent, or is 0, 1 or 2 when Z is present.

One embodiment of the invention of relates to cell cycle inhibitors of Formula 3 or 4

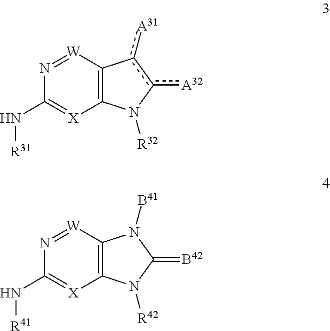

enantiomers, diastereomers, salts and solvates thereof wherein
W and X are independently CH or N;
A³¹ and A³² together with ring carbon atoms to which they are attached combine to form benzene, cyclopentadiene, pyridine, pyridone, pyrimidine, pyrazine, pyridazine, 2H-pyran, pyrrole, imidazole, pyrazole, triazole, furan, oxazole, isoxazole, oxadiazole, thiophene, thiazole, isothiazole or thiadiazole any of which may be optionally partially saturated, and any of which may be optionally independently substituted with one or more R^{x} groups as allowed by valance;
B⁴¹ and B⁴² together with the ring atoms to which they are attached combine to form dihydropyridine, dihydropyridone, dihydropyrimidine, dihydropyrimidone, dihydropyrazine, dihyrdopyridazine, pyrrole, imidazole, pyrazole, triazole, or tetrazole any of which may be optionally partially saturated, and any of which may be optionally independently substituted with one or more R^{x} groups as allowed by valance;
R⁴¹ and R³¹ is —Y-(alkylene)ₘ-R^{34a};
Y is alkyl, alkenyl, alkynyl, cycloalkyl, cycloalkenyl, heterocyclo, aryl or heteroaryl any of which may be optionally independently substituted with one or more R^{34x} groups as allowed by valance;
R^{34a} is heterocyclo, heteroaryl, —NR^{33c}R^{34c}, —C(═O)NR^{33c}R^{34c}; —O—R^{35c}, —S(O)ₙ—R³⁵, or —S(O)ₙ—NR^{33c}R^{34c} any of which may be optionally independently substituted with one or more R^{4x} groups as allowed by valance, and wherein two R^{34x} groups bound to the same or adjacent atom may optionally combine to form a ring;
R³² and R⁴² is alkyl, cycloalkyl, heterocyclo, aryl, —S(O)ₙR^{35c}, —C(═O)R^{35c}, —C(═S)R^{35c}, —C(═O)OR^{35c}, —C(═S)OR^{35c}, —C(═O)NR^{33c}R^{34c}, —C(═S)NR^{33c}R^{34c}, —SO₂NR^{33c}R^{34c}, any of which may be optionally independently substituted with one or more R^{x} groups as allowed by valance;
R^{33c} and R^{34c} at each occurrence are independently
   (i) hydrogen or
   (ii) alkyl, cycloalkyl, heterocyclo, aryl, heteroaryl, cycloalkylalkyl, heterocycloalkyl, arylalkyl, or heteroarylalkyl any of which may be optionally independently substituted with one or more R^{4x} groups as allowed by valance, and wherein two R^{34x} groups bound to the same or adjacent atom may optionally combine to form a ring;
or R^{33c} and R^{34c} together with the nitrogen atom to which they are attached may combine to form a heterocyclo ring optionally independently substituted with one or more R^{4x} groups as allowed by valance, and wherein two R^{4x} groups bound to the same or adjacent atom may optionally combine to form a ring;
R^{33c}* and R^{34c}* at each occurrence are independently
   (i) hydrogen or
   (ii) alkyl, alkenyl, alkynyl cycloalkyl, heterocyclo, aryl, heteroaryl, cycloalkylalkyl, heterocycloalkyl, arylalkyl, or heteroarylalkyl any of which may be optionally independently substituted with one or more R^{x} groups as allowed by valance;
or R^{33c}* and R^{34c}* together with the nitrogen atom to which they are attached may combine to form a heterocyclo ring optionally independently substituted with one or more R^{x} groups as allowed by valance;
R^{35c} and R^{35c}* at each occurrence is
   (i) hydrogen or
   (ii) alkyl, alkenyl, alkynyl cycloalkyl, heterocyclo, aryl, heteroaryl, cycloalkylalkyl, heterocycloalkyl, arylalkyl, or heteroarylalkyl any of which may be optionally independently substituted with one or more R^{4x} groups as allowed by valance; R^{34x} at each occurrence is independently, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, -(alkylene)ₘ-OR^{35c}, -(alkylene)ₘ-S(O)ₙR^{35c}, -(alkylene)ₘ-NR^{33c}R^{34c}, -(alkylene)ₘ-C(═O)R^{35c}, -(alkylene)ₘ-C(═S)R^{35c}, -(alkylene)ₘ-C(═O)OR^{35c}, -(alkylene)ₘ-OC(═O)R^{35c}, -(alkylene)ₘ-C(═S)OR^{35c}, -(alkylene)ₘ-C(═O)NR^{33c}R^{34c}, -(alkylene)ₘ-C(═S)NR^{33c}R^{34c}, -(alkylene)ₘ-N(R^{33c})C(═O)NR^{33c}R^{34c}, (alkylene)ₘ-N(R^{33c})C(═S)NR^{33c}R^{34c}, -(alkylene)ₘ-N(R^{33c})C(═O)R^{35c}, -(alkylene)ₘ-N(R^{33c})C(═S)R^{35c}, -(alkylene)ₘ-OC(═O)NR^{33c}R^{34c}, -(alkylene)ₘ-OC(═S) NR^{33c}R^{34c}, -(alkylene)ₘ-SO₂NR^{33c}R^{34c}, -(alkylene)ₘ-N(R^{33c})SO₂R^{35c}, -(alkylene)ₘ-N(R^{33c})SO₂NR^{33c}R^{34c}, -(alkylene)ₘ-N(R^{33c})C(═O)OR^{35c}, -(alkylene)ₘ-N(R^{33c})C(═S)OR^{35c}, or -(alkylene)ₘ-N(R^{33c})SO₂R^{35c};
wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more -(alkylene)ₘ-CN, -(alkylene)ₘ-OR^{35c}*, -(alkylene)ₘ-S(O)ₙR^{35c}*, -(alkylene)ₘ-NR^{33c}*R^{34c}*, -(alkylene)ₘ-C(═O)R^{35c}*, -(alkylene)ₘ-C(═S)R^{35c}*, -(alkylene)ₘ-C(═O)O R^{35c}*, -(alkylene)ₘ-OC(═O)R^{35c}*, -(alkylene)ₘ-C(═S)OR^{35c}*, -(alkylene)ₘ-C(═O)NR^{33c}*R^{34c}*, -(alkylene)ₘ-C(═S)NR^{33c}*R^{34c}, -(alkylene)ₘ-N(R^{33c}*)C(═O)NR^{33c}*R^{34c}*, (alkylene)ₘ-N(R^{33c}*)C(═S)NR³³*R^{34c}*, -(alkylene)ₘ-N(R^{33c}*)C(═O)R^{35c}*, -(alkylene)ₘ-N(R³³*)C(═S)R^{3c} (═S)⁵c*, -(alkylene)ₘ-OC(═O)NR^{33c}R^{34c}*, -(alkylene)ₘ-OC(═S)NR^{33c}*R^{34c}*, -(alkylene)ₘ-SO₂NR^{33c}*R^{34c}*, -(alkylene)ₘ-N(R^{33c}*)SO₂R^{35c}*, -(alkylene)ₘ-N(R^{33c}*)SO₂NR^{33c}*R^{34c}*, -(alkylene)ₘ-N(R^{33c}*)C(═O)OR^{35c}*, -(alkylene)ₘ-N(R^{33c}*)C(═S)OR^{35c}*, or -(alkylene)ₘ-N(R^{33c}*)SO₂R^{35c}*;
n is independently 0, 1 or 2; and
m is independently 0 or 1.

One embodiment of the invention of relates to cell cycle inhibitors of a compound of Formula 5:

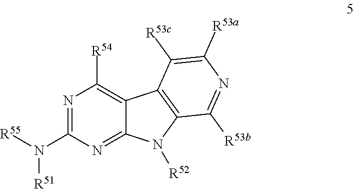

or a salt thereof, a hydrate thereof, or a mixture thereof,
wherein:
R⁵¹ is a group of Formula 5A, Formula 5B, Formula 5C, or Formula 5D

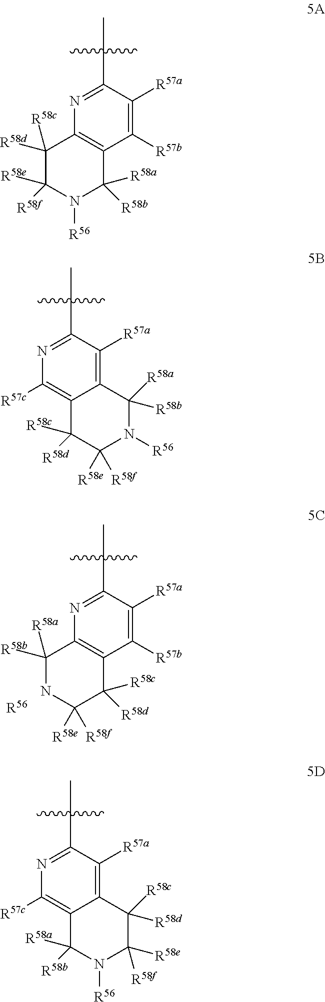

wherein the [symbol] symbol indicates the point of attachment of the group of Formula 5A, 5B, 5C, or 5D to the rest of the molecule;
R⁵² is a C₅-C₇ cycloalkyl group, is a 5 to 7-membered heterocyclyl group that includes 1, 2, or 3 heteroatoms selected from N, O, and S, or is a C₇-C₁₀ bicyclic group; wherein the C₅-C₇ cycloalkyl group, the 5 to 7 membered heterocyclyl group, or the C₇-C₁₀ bicyclic group is unsubstituted or is substituted with 1-3 substituents independently selected from unsubstituted —(C₁-C₆ alkyl), —OH, halo, —O—(C₁-C₆ alkyl), —CO₂H, —C(═O)—O—(C₁-C₆ alkyl), —C(═O)—NR′R″, —NR′R″, or a substituted —(C₁-C₄ alkyl), wherein the substituted —(C₁-C₄ alkyl) is substituted with 1-3 substituents independently selected from halo, —OH, —OCH₃, —S(═O)₂—CH₃, or —C(═O)—CH₃;
R^{53a} is selected from —H, —F, or —Cl, —(C₁-C₃ alkyl), or —O—(C₁-C₃ alkyl);
R^{53b} is —H, halo, —OH, —O—(C₁-C₆ alkyl), unsubstituted —(C₁-C₆ alkyl), —NR′R″, —C(═O)—(C₁-C₆ alkyl), —C(═O)—O—(C₁-C₆ alkyl), —C(═O)—NR′R″, or a substituted —(C₁-C₆ alkyl), wherein the substituted —(C₁-C₆ alkyl) is substituted with 1-3 substituents independently selected from halo, —OH, —OCH₃, —CN, or —NO₂;
R^{53c} is —H, —(C₁-C₃ alkyl), or halo;
R⁵⁴ is —H;
R⁵⁵ is —H;
R⁵⁶ is selected from —H, —(C₁-C₆ alkyl), —C(═O)—(C₁-C₆ alkyl), —C(═O)—O—(C₁-C₆ alkyl), —C(═O)—C(═O)—OH, —C(═O)—NR′R″, or —S(═O)—NR′R″, wherein the alkyl group of the —(C₁-C₆ alkyl), —C(═O)—(C₁-C₆ alkyl), and —C(═O)—O—(C₁-C₆ alkyl) groups is unsubstituted or is substituted with 1-3 substituents independently selected from —OH, F, —S(═O)₂—(C₁-C₆ alkyl), —O—(C₁-C₆ alkyl), —NR′R″, or —CN;
R^{57a} is-H, —CH₃, or halo;
R^{57b} is —H, —(C₁-C₆ alkyl), or halo; or R^{57b} is absent if R⁵¹ is a group of Formula 5B or Formula 5D;
R^{57c} is —H, unsubstituted —(C₁-C₆ alkyl), halo, —O—(C₁-C₆ alkyl), —NO₂, —CN, —NR′R″, —CO₂H, —C(═O)—O—(C₁-C₆ alkyl), —C(═O)—NR′R″, or a substituted —(C₁-C₆ alkyl), wherein the substituted —(C₁-C₆ alkyl) is substituted with 1-3 substituents independently selected from —OH, halo, —O—(C₁-C₆ alkyl), —CN, —NR′R″, or —S(═O)₂—CH₃; or R^{57c} is absent if R⁵¹ is a group of Formula 5A or Formula 5C;
R^{58a} is —H, unsubstituted —(C₁-C₆ alkyl), or a substituted —(C₁-C₆ alkyl), wherein the substituted —(C₁-C₆ alkyl) is substituted with 1-3 substituents independently selected from —OH, halo, or —O—(C₁-C₆ alkyl);
R^{58b} is —H, unsubstituted —(C₁-C₆ alkyl), or a substituted —(C₁-C₆ alkyl), wherein the substituted —(C₁-C₆ alkyl) is substituted with 1-3 substituents independently selected from —OH, halo, or —O—(C₁-C₆ alkyl); or R^{8a} and R^{8b}, when taken together, can represent ═O;
R^{58c} is selected from —H, —OH, unsubstituted —(C₁-C₆ alkyl), or a substituted —(C₁-C₆ alkyl), wherein the substituted —(C₁-C₆ alkyl) is substituted with 1-3 substituents independently selected from —OH, halo, or —O—(C₁-C₆ alkyl);
R^{58d} is —H, unsubstituted —(C₁-C₆ alkyl), or a substituted —(C₁-C₆ alkyl), wherein the substituted —(C₁-C₆ alkyl) is substituted with 1-3 substituents independently selected from —OH, halo, or —O—(C₁-C₆ alkyl);
R^{58e} is —H, unsubstituted —(C₁-C₆ alkyl), or a substituted —(C₁-C₆ alkyl), wherein the substituted —(C₁-C₆ alkyl) is substituted with 1-3 substituents independently selected from —OH, halo, or —O—(C₁-C₆ alkyl);
R^{58f} is —H, unsubstituted —(C₁-C₆ alkyl), or a substituted —(C₁-C₆ alkyl), wherein the substituted —(C₁-C₆ alkyl) is substituted with 1-3 substituents independently selected from —OH, halo, or —O—(C₁-C₆ alkyl); or R^{58e} and R^{58f}, when taken together, can represent ═O; and
R′ and R″ are independently selected from —H, unsubstituted —(C₁-C₄ alkyl), or —(C₁-C₄ alkyl) substituted with 1 to 3 substituents independently selected from —OH or —F.

One embodiment of the invention of relates to cell cycle inhibitors of Formula 6

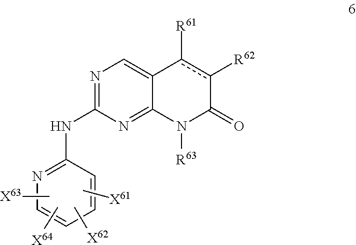

### Wherein

X⁶¹, X⁶², and X⁶³ are independently hydrogen, halogen, C₁-C₆ alkyl, C₁-C₆ haloalkyl, C₁-C₈ alkoxy, C₁-C₈ alkoxyalkyl, CN, NO₂, OR⁶⁵, NR⁶⁵R⁶⁶, CO₂R⁶⁵, COR⁶⁵, S(O)R⁶⁵, CONR⁶⁵R⁶⁶, NR⁶⁵COR⁶⁶, NR⁶⁵SO₂R⁶⁶, SO₂NR⁵R⁶, and P(O)(OR⁶⁵)(OR⁶⁶); with the proviso that at least one of X⁶¹, X⁶², and X⁶³ must be hydrogen;
R⁶¹ is, in each instance, independently, hydrogen, halogen, C₁-C₆ alkyl, C₁-C₆ haloalkyl, C₁-C₆ hydroxyalkyl, or C₃-C₇cycloalkyl;
R⁶² and R⁶⁴ are independently selected from hydrogen, halogen, C₁-C₈ alkyl, C₃-C₇ cycloalkyl, C₁-C₈ alkoxy, C₁-C₈ alkoxyalkyl, C₁-C₈ haloalkyl, C₁-C₈ hydroxyalkyl, C₂-C₈ alkenyl, C₂-C₈ alkynyl, nitrile, nitro, OR⁵, SR⁶⁵, NR⁶⁵R⁶⁶, N(O)R⁶⁵R⁶⁶, P(O)(OR⁶⁵)(OR⁶⁶), (CR⁶⁵R⁶⁶)ₘNR⁶⁷R⁶⁸, COR⁶⁵, (CR⁶⁴R⁶⁵)ₘC(O)R⁶⁷, CO₂R⁶⁵, CONR⁶⁵R⁶⁶, C(O)NR⁵SO₂R⁶, NR⁶⁵SO₂R⁶⁶, C(O)NR⁶⁵OR⁶⁶, S(O)ₙR⁶⁵, SO₂NR⁶⁵R⁶⁶, P(O)(OR⁶⁵)(OR⁶⁶), (CR⁶⁵R⁶⁶)ₘP(O)(R⁶⁷)(OR⁶⁸), (CR⁶⁵R⁶⁶)ₘ-aryl, (CR⁶⁵R⁶⁶)ₘ-heteroaryl, -T(CH₂)ₘQR⁶⁵, —C(O)T(CH₂)ₘQR⁶⁵, NR⁶⁵C(O)T(CH₂)ₘQR⁶⁵, and —CR⁶⁵═CR⁶⁶C(O)R⁶⁷; or
R⁶¹ and R⁶² may form a carbocyclic group containing 3-7 ring members, preferably 5-6 ring members, up to four of which can optionally be replaced with a heteroatom independently selected from oxygen, sulfur, and nitrogen, and wherein the carbocyclic group is unsubstituted or substituted with one, two, or three groups independently selected from halogen, hydroxy, hydroxyalkyl, nitrile, lower C₁-C₈ alkyl, lower C₁-C₈ alkoxy, alkoxycarbonyl, alkylcarbonyl, alkylcarbonylamino, aminoalkyl, trifluoromethyl, N-hydroxyacetamide, trifluoromethylalkyl, amino, and mono or dialkylamino, (CH₂)ₘC(O)NR⁶⁵R⁶⁶, and O(CH₂)ₘC(O)OR⁶⁵, provided, however, that there is at least one carbon atom in the carbocyclic ring and that if there are two or more ring oxygen atoms, the ring oxygen atoms are not adjacent to one another;
T is O, S, NR⁶⁷, N(O)R⁶⁷, NR⁶⁷R⁶⁸W, or CR⁶⁷R⁶⁸;
Q is O, S, NR⁶⁷, N(O)R⁶⁷, NR⁶⁷R⁶⁸W, CO₂, O(CH₂)ₘ-heteroaryl, O(CH₂)ₘS(O)ₙR⁶⁸, (CH₂)-heteroaryl, or a carbocyclic group containing from 3-7 ring members, up to four of which ring members are optionally heteroatoms independently selected from oxygen, sulfur, and nitrogen, provided, however, that there is at least one carbon atom in the carbocyclic ring and that if there are two or more ring oxygen atoms, the ring oxygen atoms are not adjacent to one another, wherein the carbocyclic group is unsubstituted or substituted with one, two, or three groups independently selected from halogen, hydroxy, hydroxyalkyl, lower alkyl, lower alkoxy, alkoxycarbonyl, alkylcarbonyl, alkylcarbonylamino, aminoalkyl, trifluoromethyl, N-hydroxyacetamide, trifluoromethylalkyl, amino, and mono or dialkylamino;
W is an anion selected from the group consisting of chloride, bromide, trifluoroacetate, and triethylammonium;
m=0-6;
n=0-2;
R⁶⁴ and one of X⁶¹, X⁶² and X⁶³ may form an aromatic ring containing up to three heteroatoms independently selected from oxygen, sulfur, and nitrogen, and optionally substituted by up to 4 groups independently selected from halogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethyialkylaminoalkyl, amino, mono- or dialkylamino, N-hydroxyacetamido, aryl, heteroaryl, carboxyalkyl, nitrile, NR⁶⁷SO₂R⁶⁸, C(O)NR⁶⁷R⁶⁸, NR⁶⁷C(O)R⁶⁸, C(O)OR⁶⁷, C(O)NR⁶⁷SO₂R⁶⁸, (CH₂)ₘS(O)ₙR⁶⁷, (CH₂)ₘ-heteroaryl, O(CH₂)ₘ-heteroaryl, (CH₂)ₘC(O)NR⁶⁷R⁶⁸, O(CH₂)ₘC(O)OR⁶⁷, (CH₂)ₘSO₂NR⁶⁷R⁶⁸, and C(O)R⁶⁷;
R⁶³ is hydrogen, aryl, C₁-C₈ alkyl, C₁-C₈ alkoxy, C₃-C₇ cycloalkyl, or C₃-C₇-heterocyclyl;
R⁶⁵ and R⁶⁶ independently are hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl; or
R⁶⁵ and R⁶⁶, when attached to the same nitrogen atom, taken together with the nitrogen to which they are attached, form a heterocyclic ring containing from 3-8 ring members, up to four of which members can optionally be replaced with heteroatoms independently selected from oxygen, sulfur, S(O), S(O)₂, and nitrogen, provided, however, that there is at least one carbon atom in the heterocyclic ring and that if there are two or more ring oxygen atoms, the ring oxygen atoms are not adjacent to one another, wherein the heterocyclic group is unsubstituted or substituted with one, two or three groups independently selected from halogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, nitrile, mono- or dialkylamino, N-hydroxyacetamido, aryl, heteroaryl, carboxyalkyl, NR⁶⁷SO₂R⁶⁸, C(O)NR⁶⁷R⁶⁸, NR⁶⁷C(O)R⁶⁸, C(O)OR⁶⁷, C(O)NR⁶⁷SO₂R⁶⁸, (CH₂)ₘS(O)ₙR⁶⁷, (CH₂)ₘ-heteroaryl, O(CH₂)ₘ-heteroaryl, (CH₂)ₘC(O)NR⁶⁷R⁶⁸, O(CH₂)ₘC(O)OR⁶⁷, and (CH₂)SO₂NR⁶⁷R⁶⁸;
R⁶⁷ and R⁶⁸ are, independently, hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl; or
R⁶⁷ and R⁶⁸, when attached to the same nitrogen atom, taken together with the nitrogen to which they are attached, may form a heterocyclic ring containing from 3-8 ring members, up to four of which members are optionally heteroatoms independently selected from oxygen, sulfur, S(O), S(O)₂, and nitrogen, provided, however, that there is at least one carbon atom in the heterocyclic ring and that if there are two or more ring oxygen atoms, the ring oxygen atoms are not adjacent to one another, wherein the heterocyclic group is unsubstituted or substituted with one, two or three groups independently selected from halogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, nitrile, mono- or dialkylamino, N-hydroxyacetamido, aryl, heteroaryl, carboxyalkyl;
   and the salts, esters, and amides, thereof.

One embodiment of the invention of relates to cell cycle inhibitors of Formula 7

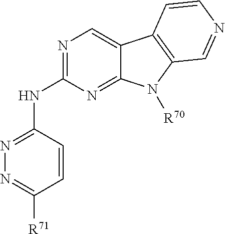

Wherein R⁷⁰ is cyclopentyl or 4-methylcyclohexyl; and R⁷¹ is 3-methylpiperazin-1-yl, 4-(dimethylamino)piperidin-1-yl or 2,2-dioxo-2-thia-8-azaspiro[4.5]decan-8-yl; and salts thereof.

One embodiment of the invention of relates to cell cycle inhibitors of the formulas

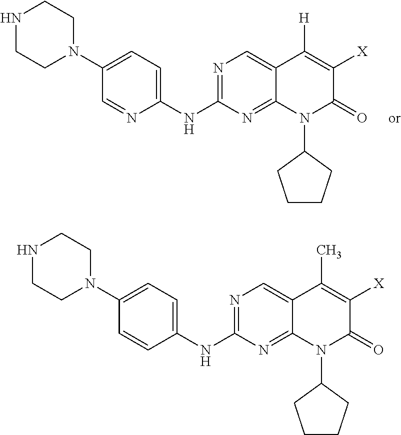

Wherein X is Br, Et, or NH₂; and salts, thereof.

One embodiment of the invention of relates to cell cycle inhibitors of the formula

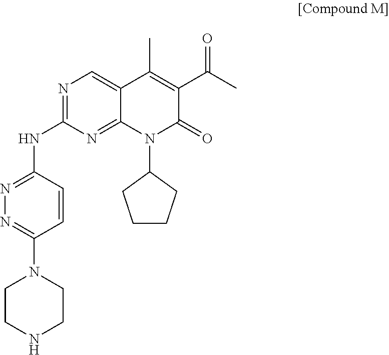 [SMILES: CC(=O)c1c(C)c2cnc(Nc3ccc(N4CCNCC4)nn3)nc2n(C2CCCC2)c1=O]

and salts thereof.

One embodiment of the invention of relates to cell cycle inhibitors selected from Pfizer PD 332991 [6-Acetyl-8-cyclopentyl-5-methyl-2-[[5-(piperazin-1-yl)pyridin-2-yl]amino]-8H-pyrido[2,3-d]pyrimidin-7-one], Lilly LY 2835219, Astex AT 9311, and Piramal Life Sciences P 276-00.

One embodiment of the invention of relates to cell cycle inhibitors selected from
9-cyclopentyl-N-(6-(4-(dimethylamino)piperidin-1-yl)pyridazin-3-yl)-9H-pyrido[4′,3′:4,5]pyrrolo[2,3-d]pyrimidin-2-amine [Compound C];
9-(4-methylcyclohexyl)-N-(6-(3-methylpiperazin-1-yl)pyridazin-3-yl)-9H-pyrido[4′,3′:4,5]pyrrolo[2,3-d]pyrimidin-2-amine [Compound D];
8-(6-((9-(4-methylcyclohexyl)-9H-pyrido[4′,3′:4,5]pyrrolo[2,3-d]pyrimidin-2-yl)amino)pyridazin-3-yl)-2-thia-8-azaspiro[4.5]decane 2,2-dioxide (Compound E);
N-(6-(4-(dimethylamino)piperidin-1-yl)pyridin-3-yl)-4,4-dimethylspiro[cyclohex[2]ene-1,9′-pyrido[4′,3′:3,4]cyclopenta[1,2-d]pyrimidin]-2′-amine (Compound F);
1-(6-((9-cyclopentyl-9H-pyrido[4′,3′:4,5]pyrrolo[2,3-d]pyrimidin-2-yl)amino)pyridazin-3-yl)pyrrolidin-3-ol (Compound G);
N-(6-(4-(dimethylamino)piperidin-1-yl)pyridazin-3-yl)-4,4-dimethylspiro[cyclohex[2]ene-1,9′-pyrido[4′,3′:3,4]cyclopenta[1,2-d]pyrimidin]-2′-amine (Compound H);
9-(2,4-difluorophenyl)-N-(5-(piperazin-1-yl)pyridin-2-yl)-9H-pyrido[4′,3′:4,5]pyrrolo[2,3-d]pyrimidin-2-amine (Compound I);
9-(2,4-difluorophenyl)-N-(5-(3,3-dimethylpiperazin-1-yl)pyridin-2-yl)-9H-pyrido[4′,3′:4,5]pyrrolo[2,3-d]pyrimidin-2-amine (Compound J);
4,4-dimethyl-N-(6-(piperazin-1-yl)pyridazin-3-yl)spiro[cyclohex[2]ene-1,9′-pyrido[4′,3′:3,4]cyclopenta[1,2-d]pyrimidin]-2′-amine (Compound K);
N-(5-(4-(dimethylamino)piperidin-1-yl)pyridin-2-yl)-4,4-dimethylspiro[cyclohex[2]ene-1,9′-pyrido[4′,3′:3,4]cyclopenta[1,2-d]pyrimidin]-2′-amine (Compound L);
4-[6-[(6-bromo-8-cyclopentyl-7,8-dihydro-7-oxopyrido[2,3-d]pyrimidin-2-yl)amino]-3-pyridinyl]-1-piperazinecarboxylic acid, 1,1-dimethylethyl ester;
4-[6-[(8-cyclopentyl-6-ethyl-7,8-dihydro-7-oxopyrido[2,3-d]pyrimidin-2-yl)amino]-3-pyridinyl]-1-piperazinecarboxylic acid, 1,1-dimethylethyl ester;
8-cyclopentyl-2-[[5-[4-[(1,1-dimethylethoxy)carbonyl]-1-piperazinyl]-2-pyridinyl]amino]-7,8-dihydro-7-oxo-pyrido[2,3-d]pyrimidine-6-carboxylic acid, ethyl ester;
4-[6-[[8-cyclopentyl-6-[[(1,1-dimethylethoxy)carbonyl]amino]-7,8-dihydro-7-oxopyrido[2,3-d]pyrimidin-2-yl]amino]-3-pyridinyl]-1-piperazinecarboxylic acid, 1,1-dimethylethyl ester;
4-[6-[(6-bromo-8-cyclopentyl-7,8-dihydro-5-methyl-7-oxopyrido[2,3-d]pyrimidin-2-yl)amino]-3-pyridinyl]-1-piperazinecarboxylic acid, 1,1-dimethylethyl ester;
4-[6-[(8-cyclopentyl-6-fluoro-7,8-dihydro-7-oxopyrido[2,3-d]pyrimidin-2-yl)amino]-3-pyridinyl]-1-piperazinecarboxylic acid, 1,1-dimethylethyl ester;
4-[6-[(8-cyclopentyl-7,8-dihydro-6-methyl-7-oxopyrido[2,3-d]pyrimidin-2-yl)amino]-3-pyridinyl]-1-piperazinecarboxylic acid, 1,1-dimethylethyl ester;
4-[6-[[8-cyclopentyl-7,8-dihydro-7-oxo-6-(phenylmethyl)pyrido[2,3-d]pyrimidin-2-yl]amino]-3-pyridinyl]-1-piperazinecarboxylic acid, 1,1-dimethylethyl ester;
4-[6-[[6-[(acetyloxy)methyl]-8-cyclopentyl-7,8-dihydro-7-oxopyrido[2,3-d]pyrimidin-2-yl]amino]-3-pyridinyl]-1-piperazinecarboxylic acid, 1,1-dimethylethyl ester;
8-cyclopentyl-2-[[5-[4-[(1,1-dimethylethoxy)carbonyl]-1-piperazinyl]-2-pyridinyl]amino]-7,8-dihydro-5-methyl-7-oxo-pyrido[2,3-d]pyrimidine-6-carboxylic acid, ethyl ester;
4-[6-[(6-acetyl-8-cyclopentyl-7,8-dihydro-7-oxopyrido[2,3-d]pyrimidin-2-yl)amino]-3-pyridinyl]-1-piperazinecarboxylic acid, 1,1-dimethylethyl ester;
4-[6-[[8-cyclopentyl-6-(1-ethoxyethenyl)-7,8-dihydro-5-methyl-7-oxopyrido[2,3-d]pyrimidin-2-yl]amino]-3-pyridinyl]-1-piperazinecarboxylic acid, 1,1-dimethylethyl ester;
4-[6-[[8-cyclopentyl-6-(2-ethoxyethoxy)-7,8-dihydro-7-oxopyrido[2,3-d]pyrimidin-2-yl]amino]-3-pyridinyl]-1-piperazinecarboxylic acid, 1,1-dimethylethyl ester;
2-[[5-[bis(2-methoxyethyl)amino]-2-pyridinyl]amino]-6-bromo-8-cyclopentyl-5-methyl-pyrido[2,3-d]pyrimidin-7(8H)-one;
4-[6-[(8-cyclopentyl-7,8-dihydro-7-oxopyrido[2,3-d]pyrimidin-2-yl)amino]-3-pyridinyl]-1-piperazinecarboxylic acid, 1,1-dimethylethyl ester;
4-[6-[(8-cyclohexyl-7,8-dihydro-7-oxopyrido[2,3-d]pyrimidin-2-yl)amino]-3-pyridinyl]-1-piperazinecarboxylic acid, 1,1-dimethylethyl ester;
4-[6-[(8-cyclopropyl-7,8-dihydro-7-oxopyrido[2,3-d]pyrimidin-2-yl)amino]-3-pyridinyl]-1-piperazinecarboxylic acid, 1,1-dimethylethyl ester;
[1-[6-[[8-cyclopentyl-6-(1-ethoxyethenyl)-7,8-dihydro-5-methyl-7-oxopyrido[2,3-d]pyrimidin-2-yl]amino]-3-pyridinyl]-3-pyrrolidinyl]-carbamic acid, 1,1-dimethylethyl ester;
6-bromo-8-cyclopentyl-2-[[5-(4-hydroxy-1-piperidinyl)-2-pyridinyl]amino]-5-methyl-pyrido[2,3-d]pyrimidin-7(8H)-one;
8-cyclopentyl-6-(1-ethoxyethenyl)-2-[[5-(4-hydroxy-1-piperidinyl)-2-pyridinyl]amino]-5-methyl-pyrido[2,3-d]pyrimidin-7(8H)-one;
6-acetyl-8-cyclopentyl-2-[[5-(4-hydroxy-1-piperidinyl)-2-pyridinyl]amino]-5-methyl-pyrido[2,3-d]pyrimidin-7(8H)-one;
4-[6-[(6-bromo-8-cyclopentyl-7,8-dihydro-5-methyl-7-oxopyrido[2,3-d]pyrimidin-2-yl)amino]-3-pyridinyl]hexahydro-1H-azepine-1-carboxylic acid, 1,1-dimethylethyl ester;
4-[6-[[8-cyclopentyl-6-(1-ethoxyethenyl)-7,8-dihydro-5-methyl-7-oxopyrido[2,3-d]pyrimidin-2-yl]amino]-3-pyridinyl]hexahydro-1H-1,4-diazepine-1-carboxylic acid, 1,1-dimethylethyl ester;
4-[6-[(8-cyclopentyl-7,8-dihydro-5-methyl-7-oxopyrido[2,3-d]pyrimidin-2-yl)amino]-3-pyridinyl]-1-piperazinecarboxylic acid, 1,1-dimethylethyl ester;
4-[6-[(6-bromo-8-cyclopentyl-7,8-dihydro-5-methyl-7-oxopyrido[2,3-d]pyrimidin-2-yl)amino]-3-pyridinyl]-2,2-dimethyl-1-piperazinecarboxylic acid, 1,1-dimethylethyl ester;
4-[6-[[8-cyclopentyl-6-(1-ethoxyethenyl)-7,8-dihydro-5-methyl-7-oxopyrido[2,3-d]pyrimidin-2-yl]amino]-3-pyridinyl]-2,2-dimethyl-1-piperazinecarboxylic acid, 1,1-dimethylethyl ester;
4-[6-[(6-bromo-8-cyclopentyl-7,8-dihydro-5-methyl-7-oxopyrido[2,3-d]pyrimidin-2-yl)amino]-3-pyridinyl]-2,6-dimethyl-1-piperazinecarboxylic acid, 1,1-dimethylethyl ester;
6-bromo-8-cyclopentyl-5-methyl-2-[[5-(4-morpholinyl)-2-pyridinyl]amino]-pyrido[2,3-d]pyrimidin-7(8H)-one;
8-cyclopentyl-6-(1-ethoxyethenyl)-5-methyl-2-[[5-(1-piperidinyl)-2-pyridinyl]amino]-pyrido[2,3-d]pyrimidin-7(8H)-one;
4-[6-[[8-cyclopentyl-7,8-dihydro-6-[(2-methoxyethoxy)methyl]-7-oxopyrido[2,3-d]pyrimidin-2-yl]amino]-3-pyridinyl]-1-piperazinecarboxylic acid, 1,1-dimethylethyl ester;
8-cyclopentyl-2-[[5-(2,6-dimethyl-4-morpholinyl)-2-pyridinyl]amino]-6-(1-ethoxyethenyl)-5-methyl-Pyrido[2,3-d]pyrimidin-7(8H)-one;
8-cyclopentyl-2-(2-pyridinylamino)-pyrido[2,3-d]pyrimidin-7(8H)-one;
6-bromo-8-cyclopentyl-2-[[5-(1-piperazinyl)-2-pyridinyl]amino]-pyrido[2,3-d]pyrimidin-7(8H)-one, hydrochloride;
8-cyclopentyl-6-ethyl-2-[[5-(1-piperazinyl)-2-pyridinyl]amino]-pyrido[2,3-d]pyrimidin-7(8H)-one, hydrochloride;
8-cyclopentyl-7,8-dihydro-7-oxo-2-[[5-(1-piperazinyl)-2-pyridinyl]amino]-pyrido[2,3-d]pyrimidine-6-carboxylic acid, ethyl ester, hydrochloride;
6-amino-8-cyclopentyl-2-[[5-(1-piperazinyl)-2-pyridinyl]amino]-pyrido[2,3-d]pyrimidin-7(8H)-one, hydrochloride;
6-bromo-8-cyclopentyl-2-[[5-(1-methyl-2-pyrrolidinyl)-2-pyridinyl]amino]-pyrido[2,3-d]pyrimidin-7(8H)-one, hydrochloride;
6-bromo-8-cyclohexyl-2-(2-pyridinylamino)-pyrido[2,3-d]pyrimidin-7(8H)-one;
7-bromo-5-cyclopentyl-8-methyl-3-[[5-(1-piperazinyl)-2-pyridinyl]amino]-pyrido[2,3-d]pyrimidin-7(8H)-one;
8-cyclopentyl-6-fluoro-2-[[5-(1-piperazinyl)-2-pyridinyl]amino]-pyrido[2,3-d]pyrimidin-7(8H)-one, hydrochloride;
8-cyclopentyl-6-methyl-2-[[5-(1-piperazinyl)-2-pyridinyl]amino]-pyrido[2,3-d]pyrimidin-7(8H)-one, hydrochloride;
4-[6-[[8-cyclopentyl-7,8-dihydro-6-(2-methylpropoxy)-7-oxopyrido[2,3-d]pyrimidin-2-yl]amino]-3-pyridinyl]-1-piperazinecarboxylic acid, 1,1-dimethylethyl ester;
8-cyclopentyl-6-(2-methylpropoxy)-2-[[5-(1-piperazinyl)-2-pyridinyl]amino]-pyrido[2,3-d]pyrimidin-7(8H)-one, hydrochloride (1:2) 8-cyclopentyl-6-(phenylmethyl)-2-[[5-(1-piperazinyl)-2-pyridinyl]amino]-pyrido[2,3-d]pyrimidin-7(8H)-one, hydrochloride;
8-cyclopentyl-6-(hydroxymethyl)-2-[[5-(1-piperazinyl)-2-pyridinyl]amino]-pyrido[2,3-d]pyrimidin-7(8H)-one, hydrochloride;
8-cyclopentyl-7,8-dihydro-5-methyl-7-oxo-2-[[5-(1-piperazinyl)-2-pyridinyl]amino]-pyrido[2,3-d]pyrimidine-6-carboxylic acid, ethyl ester, hydrochloride;
6-acetyl-8-cyclopentyl-2-[[5-(1-piperazinyl)-2-pyridinyl]amino]-pyrido[2,3-d]pyrimidin-7(8H)-one, hydrochloride;
6-acetyl-8-cyclopentyl-5-methyl-2-[[5-(1-piperazinyl)-2-pyridinyl]amino]-pyrido[2,3-d]pyrimidin-7(8H)-one, hydrochloride;
6-bromo-8-cyclopentyl-5-methyl-2-(2-pyridinylamino)-pyrido[2,3-d]pyrimidin-7(8H)-one;
6-bromo-8-cyclopentyl-2-(2-pyridinylamino)-pyrido[2,3-d]pyrimidin-7(8H)-one;
4-[6-[(8-cyclopentyl-7,8-dihydro-6-iodo-5-methyl-7-oxopyrido[2,3-d]pyrimidin-2-yl)amino]-3-pyridinyl]-1-piperazinecarboxylic acid, 1,1-dimethylethyl ester;
8-cyclopentyl-6-iodo-5-methyl-2-[[5-(1-piperazinyl)-2-pyridinyl]amino]-pyrido[2,3-d]pyrimidin-7(8H)-one;
8-cyclopentyl-6-ethyl-2-[[5-(4-hydroxy-1-piperidinyl)-2-pyridinyl]amino]-pyrido[2,3-d]pyrimidin-7(8H)-one;
8-cyclopentyl-6-(2-ethoxyethoxy)-2-[[5-(1-piperazinyl)-2-pyridinyl]amino]-pyrido[2,3-d]pyrimidin-7(8H)-one, hydrochloride;
6-acetyl-2-[[5-[bis(2-methoxyethyl)amino]-2-pyridinyl]amino]-8-cyclopentyl-5-methyl-pyrido[2,3-d]pyrimidin-7(8H)-one;
4-[6-[[7,8-dihydro-8-(1-methylethyl)-7-oxopyrido[2,3-d]pyrimidin-2-yl]amino]-3-pyridinyl]-1-piperazinecarboxylic acid, 1,1-dimethylethyl ester;
8-(1-methylethyl)-2-[[5-(1-piperazinyl)-2-pyridinyl]amino]-pyrido[2,3-d]pyrimidin-7(8H)-one, hydrochloride;
8-cyclopentyl-2-[[5-(1-piperazinyl)-2-pyridinyl]amino]-pyrido[2,3-d]pyrimidin-7(8H)-one, hydrochloride;
8-cyclohexyl-2-[[5-(1-piperazinyl)-2-pyridinyl]amino]-pyrido[2,3-d]pyrimidin-7(8H)-one, hydrochloride;
2-[(6-amino-2-pyridinyl)amino]-6-bromo-8-cyclopentyl-pyrido[2,3-d]pyrimidin-7(8H)-one;
2-[(6-amino-2-pyridinyl)amino]-6-bromo-8-cyclopentyl-5-methyl-pyrido[2,3-d]pyrimidin-7(8H)-one;
6-bromo-8-cyclopentyl-5-methyl-2-[[5-(4-methyl-1-piperazinyl)-2-pyridinyl]amino]-pyrido[2,3-d]pyrimidin-7(8H)-one;
8-cyclopentyl-6-(1-ethoxyethenyl)-5-methyl-2-[[5-(4-methyl-1-piperazinyl)-2-pyridinyl]amino]-pyrido[2,3-d]pyrimidin-7(8H)-one;
6-acetyl-8-cyclopentyl-5-methyl-2-[[5-(4-methyl-1-piperazinyl)-2-pyridinyl]amino]-pyrido[2,3-d]pyrimidin-7(8H)-one;
6-acetyl-2-[[5-(3-amino-1-pyrrolidinyl)-2-pyridinyl]amino]-8-cyclopentyl-5-methyl-pyrido[2,3-d]pyrimidin-7(8H)-one;
6-bromo-8-cyclopentyl-2-[[5-(hexahydro-1H-1,4-diazepin-1-yl)-2-pyridinyl]amino]-5-methyl-pyrido[2,3-d]pyrimidin-7(8H)-one, hydrochloride;
6-acetyl-8-cyclopentyl-2-[[5-(hexahydro-1H-1,4-diazepin-1-yl)-2-pyridinyl]amino]-5-methyl-pyrido[2,3-d]pyrimidin-7(8H)-one, hydrochloride;
6-acetyl-8-cyclopentyl-5-methyl-2-(2-pyridinylamino)-pyrido[2,3-d]pyrimidin-7(8H)-one;
8-cyclopentyl-5-methyl-2-[[5-(1-piperazinyl)-2-pyridinyl]amino]-pyrido[2,3-d]pyrimidin-7(8H)-one, hydrochloride;
6-bromo-8-cyclopentyl-2-[[5-(3,3-dimethyl-1-piperazinyl)-2-pyridinyl]amino]-5-methyl-pyrido[2,3-d]pyrimidin-7(8H)-one, hydrochloride;
6-acetyl-8-cyclopentyl-2-[[5-(3,3-dimethyl-1-piperazinyl)-2-pyridinyl]amino]-5-methyl-pyrido[2,3-d]pyrimidin-7(8H)-one, hydrochloride;
6-bromo-8-cyclopentyl-2-[[5-(3,5-dimethyl-1-piperazinyl)-2-pyridinyl]amino]-5-methyl-pyrido[2,3-d]pyrimidin-7(8H)-one, hydrochloride;
4-[6-[[8-cyclopentyl-6-(1-ethoxyethenyl)-7,8-dihydro-5-methyl-7-oxopyrido[2,3-d]pyrimidin-2-yl]amino]-3-pyridinyl]-2,6-dimethyl-1-piperazinecarboxylic acid, 1,1-dimethylethyl ester;
6-acetyl-8-cyclopentyl-2-[[5-(3, 5-dimethyl-1-piperazinyl)-2-pyridinyl]amino]-5-methyl-pyrido[2,3-d]pyrimidin-7(8H)-one, hydrochloride;
8-cyclopentyl-6-(1-ethoxyethenyl)-5-methyl-2-[[5-(4-morpholinyl)-2-pyridinyl]amino]-pyrido[2,3-d]pyrimidin-7(8H)-one;
6-acetyl-8-cyclopentyl-5-methyl-2-[[5-(4-morpholinyl)-2-pyridinyl]amino]-pyrido[2,3-d]pyrimidin-7(8H)-one;
6-bromo-8-cyclopentyl-5-methyl-2-[[5-(1-piperidinyl)-2-pyridinyl]amino]-pyrido[2,3-d]pyrimidin-7(8H)-one;
6-acetyl-8-cyclopentyl-5-methyl-2-[[5-(1-piperidinyl)-2-pyridinyl]amino]-pyrido[2,3-d]pyrimidin-7(8H)-one;
4-[6-[[8-cyclopentyl-6-(2-ethoxy ethyl)-7,8-dihydro-7-oxopyrido[2,3-d]pyrimidin-2-yl]amino]-3-pyridinyl]-1-piperazinecarboxylic acid, 1,1-dimethylethyl ester;
8-cyclopentyl-6-(2-ethoxyethyl)-2-[[5-(1-piperazinyl)-2-pyridinyl]amino]-pyrido[2,3-d]pyrimidin-7(8H)-one, hydrochloride;
8-cyclopentyl-6-[(2-methoxyethoxy)methyl]-2-[[5-(1-piperazinyl)-2-pyridinyl]amino]-pyrido[2,3-d]pyrimidin-7(8H)-one, hydrochloride;
8-cyclopentyl-6-(ethoxymethyl)-2-(methylthio)-pyrido[2,3-d]pyrimidin-7(8H)-one;
4-[6-[[8-cyclopentyl-6-(ethoxymethyl)-7,8-dihydro-7-oxopyrido[2,3-d]pyrimidin-2-yl]amino]-3-pyridinyl]-1-piperazinecarboxylic acid, 1,1-dimethylethyl ester;
8-cyclopentyl-6-(ethoxymethyl)-2-[[5-(1-piperazinyl)-2-pyridinyl]amino]-pyrido[2,3-d]pyrimidin-7(8H)-one;
4-[6-[[8-cyclopentyl-7,8-dihydro-6-(methoxymethyl)-7-oxopyrido[2,3-d]pyrimidin-2-yl]amino]-3-pyridinyl]-1-piperazinecarboxylic acid, 1,1-dimethylethyl ester;
6-bromo-8-cyclopentyl-2-[[5-(2,6-dimethyl-4-morpholinyl)-2-pyridinyl]amino]-5-methyl-pyrido[2,3-d]pyrimidin-7(8H)-one;
8-cyclopentyl-6-(ethoxymethyl)-2-[[5-(1-piperidinyl)-2-pyridinyl]amino]pyrido[2,3-d]pyrimidin-7(8H)-one;
8-cyclopentyl-6-(ethoxymethyl)-2-[[5-(4-morpholinyl)-2-pyridinyl]amino]-pyrido[2,3-d]pyrimidin-7(8H)-one;
[[8-cyclopentyl-7,8-dihydro-7-oxo-2-[[5-(1-piperidinyl)-2-pyridinyl]amino]pyrido[2,3-d]pyrimidin-6-yl]methyl]-carbamic acid, phenylmethyl ester;
6-acetyl-8-cyclopentyl-2-[[5-(2,6-dimethyl-4-morpholinyl)-2-pyridinyl]amino]-5-methyl-pyrido[2,3-d]pyrimidin-7(8H)-one;
6-bromo-8-cyclopentyl-2-[[5-[(2R)-1-methyl-2-pyrrolidinyl]-2-pyridinyl]amino]-pyrido[2,3-d]pyrimidin-7(8H)-one, hydrochloride;
6-acetyl-8-cyclopentyl-2-[[5-(3,5-dimethyl-1-piperazinyl)-2-pyridinyl]amino]-5-methyl-pyrido[2,3-d]pyrimidin-7(8H)-one;
6-acetyl-8-cyclopentyl-2-[[5-(3,3-dimethyl-1-piperazinyl)-2-pyridinyl]amino]-5-methyl-pyrido[2,3-d]pyrimidin-7(8H)-one;
6-bromo-8-cyclopentyl-2-[[5-(hexahydro-1H-1,4-diazepin-1-yl)-2-pyridinyl]amino]-5-methyl-pyrido[2,3-d]pyrimidin-7(8H)-one;
6-acetyl-8-cyclopentyl-2-[[5-(hexahydro-1H-1,4-diazepin-1-yl)-2-pyridinyl]amino]-5-methyl-pyrido[2,3-d]pyrimidin-7(8H)-one;
8-cyclopentyl-5-methyl-2-[[5-(1-piperazinyl)-2-pyridinyl]amino]-pyrido[2,3-d]pyrimidin-7(8H)-one;
6-bromo-8-cyclopentyl-2-[[5-(3,3-dimethyl-1-piperazinyl)-2-pyridinyl]amino]-5-methyl-pyrido[2,3-d]pyrimidin-7(8H)-one;
6-bromo-8-cyclopentyl-2-[[5-(3,5-dimethyl-1-piperazinyl)-2-pyridinyl]amino]-5-methyl-pyrido[2,3-d]pyrimidin-7(8H)-one;
8-cyclopentyl-6-(2-ethoxyethyl)-2-[[5-(1-piperazinyl)-2-pyridinyl]amino]-pyrido[2,3-d]pyrimidin-7(8H)-one;
8-cyclopentyl-6-[(2-methoxyethoxy)methyl]-2-[[5-(1-piperazinyl)-2-pyridinyl]amino]-pyrido[2,3-d]pyrimidin-7(8H)-one;
8-cyclopentyl-6-(methoxymethyl)-2-[[5-(1-piperazinyl)-2-pyridinyl]amino]-pyrido[2,3-d]pyrimidin-7(8H)-one;
8-cyclopentyl-5-methyl-6-(1-oxopropyl)-2-[[5-(1-piperazinyl)-2-pyridinyl]amino]-pyrido[2,3-d]pyrimidin-7(8H)-one;
6-acetyl-8-cyclopentyl-2-[[5-(1-piperazinyl)-2-pyridinyl]amino]-pyrido[2,3-d]pyrimidin-7(8H)-one;
6-acetyl-8-cyclopentyl-5-methyl-2-[[5-(1-piperazinyl)-2-pyridinyl]amino]-pyrido[2,3-d]pyrimidin-7(8H)-one;
6-bromo-8-cyclopentyl-2-[[5-(3,5-dimethyl-1-piperazinyl)-2-pyridinyl]amino]-pyrido[2,3-d]pyrimidin-7(8H)-one;
6-bromo-8-cyclopentyl-2-[[5-(3,3-dimethyl-1-piperazinyl)-2-pyridinyl]amino]-pyrido[2,3-d]pyrimidin-7(8H)-one;
6-bromo-8-cyclopentyl-2-[[5-(4-methyl-1-piperazinyl)-2-pyridinyl]amino]-pyrido[2,3-d]pyrimidin-7(8H)-one;
2-[[5-(3-amino-1-pyrrolidinyl)-2-pyridinyl]amino]-6-bromo-8-cyclopentyl-pyrido[2,3-d]pyrimidin-7(8H)-one;
6-bromo-8-cyclopentyl-2-[[5-[3-(ethylamino)-1-pyrrolidinyl]-2-pyridinyl]amino]-pyrido[2,3-d]pyrimidin-7(8H)-one;
6-bromo-8-cyclopentyl-2-[[5-(1-pyrrolidinyl)-2-pyridinyl]amino]-pyrido[2,3-d]pyrimidin-7(8H)-one;
2-[[5-[3-(1-amino-1-methylethyl)-1-pyrrolidinyl]-2-pyridinyl]amino]-6-bromo-8-cyclopentyl-pyrido[2,3-d]pyrimidin-7(8H)-one;
1-[6-[(6-bromo-8-cyclopentyl-7,8-dihydro-7-oxopyrido[2,3-d]pyrimidin-2-yl)amino]-3-pyridinyl]-L-proline;
6-bromo-8-cyclopentyl-2-[[5-[[4-(diethylamino)butyl]amino]-2-pyridinyl]amino]pyrido[2,3-d]pyrimidin-7(8H)-one;
6-acetyl-8-cyclopentyl-2-[[5-[3-(ethylamino)-1-pyrrolidinyl]-2-pyridinyl]amino]-5-methyl-pyrido[2,3-d]pyrimidin-7(8H)-one;
6-acetyl-8-cyclopentyl-5-methyl-2-[[5-(1-pyrrolidinyl)-2-pyridinyl]amino]-pyrido[2,3-d]pyrimidin-7(8H)-one;
6-acetyl-2-[[5-[3-(1-amino-1-methylethyl)-1-pyrrolidinyl]-2-pyridinyl]amino]-8-cyclopentyl-5-methyl-pyrido[2,3-d]pyrimidin-7(8H)-one;
1-[6-[(6-acetyl-8-cyclopentyl-7,8-dihydro-5-methyl-7-oxopyrido[2,3-d]pyrimidin-2-yl)amino]-3-pyridinyl]-L-Proline;
6-acetyl-8-cyclopentyl-2-[[5-[[4-(diethylamino)butyl]amino]-2-pyridinyl]amino]-5-methyl-pyrido[2,3-d]pyrimidin-7(8H)-one;
8-cyclopentyl-2-[[5-(3,5-dimethyl-1-piperazinyl)-2-pyridinyl]amino]-6-ethyl-pyrido[2,3-d]pyrimidin-7(8H)-one;
8-cyclopentyl-2-[[5-(3,3-dimethyl-1-piperazinyl)-2-pyridinyl]amino]-6-ethyl-pyrido[2,3-d]pyrimidin-7(8H)-one;
8-cyclopentyl-6-ethyl-2-[[5-(4-methyl-1-piperazinyl)-2-pyridinyl]amino]-pyrido[2,3-d]pyrimidin-7(8H)-one;
2-[[5-(3-amino-1-pyrrolidinyl)-2-pyridinyl]amino]-8-cyclopentyl-6-ethyl-pyrido[2,3-d]pyrimidin-7(8H)-one;
8-cyclopentyl-6-ethyl-2-[[5-[3-(ethylamino)-1-pyrrolidinyl]-2-pyridinyl]amino]-pyrido[2,3-d]pyrimidin-7(8H)-one;
8-cyclopentyl-6-ethyl-2-[[5-(1-pyrrolidinyl)-2-pyridinyl]amino]-pyrido[2,3-d]pyrimidin-7(8H)-one;
2-[[5-[3-(1-amino-1-methylethyl)-1-pyrrolidinyl]-2-pyridinyl]amino]-8-cyclopentyl-6-ethyl-pyrido[2,3-d]pyrimidin-7(8H)-one;
1-[6-[(8-cyclopentyl-6-ethyl-7,8-dihydro-7-oxopyrido[2,3-d]pyrimidin-2-yl)amino]-3-pyridinyl]-L-proline;
8-cyclopentyl-2-[[5-[[4-(diethylamino)butyl]amino]-2-pyridinyl]amino]-6-ethyl-pyrido[2,3-d]pyrimidin-7(8H)-one;
8-cyclopentyl-2-[[5-(3,5-dimethyl-1-piperazinyl)-2-pyridinyl]amino]-6-(phenylmethyl)-pyrido[2,3-d]pyrimidin-7(8H)-one;
8-cyclopentyl-2-[[5-(3,3-dimethyl-1-piperazinyl)-2-pyridinyl]amino]-6-(phenylmethyl)-pyrido[2,3-d]pyrimidin-7(8H)-one;
8-cyclopentyl-2-[[5-(4-methyl-1-piperazinyl)-2-pyridinyl]amino]-6-(phenylmethyl)-pyrido[2,3-d]pyrimidin-7(8H)-one;
2-[[5-(3-amino-1-pyrrolidinyl)-2-pyridinyl]amino]-8-cyclopentyl-6-(phenylmethyl)-pyrido[2,3-d]pyrimidin-7(8H)-one;
8-cyclopentyl-2-[[5-[3-(ethylamino)-1-pyrrolidinyl]-2-pyridinyl]amino]-6-(phenylmethyl)-pyrido[2,3-d]pyrimidin-7(8H)-one;
8-cyclopentyl-6-(phenylmethyl)-2-[[5-(1-pyrrolidinyl)-2-pyridinyl]amino]-pyrido[2,3-d]pyrimidin-7(8H)-one;
2-[[5-[3-(1-amino-1-methylethyl)-1-pyrrolidinyl]-2-pyridinyl]amino]-8-cyclopentyl-6-(phenylmethyl)-pyrido[2,3-d]pyrimidin-7(8H)-one;
1-[6-[[8-cyclopentyl-7,8-dihydro-7-oxo-6-(phenylmethyl)pyrido[2,3-d]pyrimidin-2-yl]amino]-3-pyridinyl]-L-proline;
8-cyclopentyl-2-[[5-[[4-(diethylamino)butyl]amino]-2-pyridinyl]amino]-6-(phenylmethy)-pyrido[2,3-d]pyrimidin-7(8H)-one;
8-cyclopentyl-2-[[5-(3,5-dimethyl-1-piperazinyl)-2-pyridinyl]amino]-6-(hydroxymethyl)-pyrido[2,3-d]pyrimidin-7(8H)-one;
8-cyclopentyl-2-[[5-(3,3-dimethyl-1-piperazinyl)-2-pyridinyl]amino]-6-(hydroxymethyl)-pyrido[2,3-d]pyrimidin-7(8H)-one;
8-cyclopentyl-6-(hydroxymethyl)-2-[[5-(4-methyl-1-piperazinyl)-2-pyridinyl]amino]-pyrido[2,3-d]pyrimidin-7(8H)-one
2-[[5-(3-amino-1-pyrrolidinyl)-2-pyridinyl]amino]-8-cyclopentyl-6-(hydroxymethyl)-pyrido[2,3-d]pyrimidin-7(8H)-one
8-cyclopentyl-2-[[5-[3-(ethylamino)-1-pyrrolidinyl]-2-pyridinyl]amino]-6-(hydroxymethyl)-pyrido[2,3-d]pyrimidin-7(8H)-one
8-cyclopentyl-6-(hydroxymethyl)-2-[[5-(1-pyrrolidinyl)-2-pyridinyl]amino]-pyrido[2,3-d]pyrimidin-7(8H)-one
2-[[5-[3-(1-amino-1-methylethyl)-1-pyrrolidinyl]-2-pyridinyl]amino]-8-cyclopentyl-6-(hydroxymethyl-pyrido[2,3-d]pyrimidin-7(8H)-one
1-[6-[[8-cyclopentyl-7,8-dihydro-6-(hydroxymethyl)-7-oxopyrido[2,3-d]pyrimidin-2-yl]amino]-3-pyridinyl]-L-Proline,
8-cyclopentyl-2-[[5-[[4-(diethylamino)butyl]amino]-2-pyridinyl]amino]-6-(hydroxymethyl)-pyrido[2,3-d]pyrimidin-7(8H)-one
6-amino-8-cyclopentyl-2-[[5-(3,5-dimethyl-1-piperazinyl)-2-pyridinyl]amino]-pyrido[2,3-d]pyrimidin-7(8H)-one
6-amino-8-cyclopentyl-2-[[5-(3,3-dimethyl-1-piperazinyl)-2-pyridinyl]amino]-pyrido[2,3-d]pyrimidin-7(8H)-one;
6-amino-8-cyclopentyl-2-[[5-(4-methyl-1-piperazinyl)-2-pyridinyl]amino]-pyrido[2,3-d]pyrimidin-7(8H)-one;
6-amino-2-[[5-(3-amino-1-pyrrolidinyl)-2-pyridinyl]amino]-8-cyclopentyl-pyrido[2,3-d]pyrimidin-7(8H)-one;
6-amino-8-cyclopentyl-2-[[5-[3-(ethylamino)-1-pyrrolidinyl]-2-pyridinyl]amino]-pyrido[2,3-d]pyrimidin-7(8H)-one;
6-amino-8-cyclopentyl-2-[[5-(1-pyrrolidinyl)-2-pyridinyl]amino]-pyrido[2,3-d]pyrimidin-7(8H)-one;
6-amino-2-[[5-[3-(1-amino-1-methylethyl)-1-pyrrolidinyl]-2-pyridinyl]amino]-8-cyclopentyl-pyrido[2,3-d]pyrimidin-7(8H)-one;
1-[6-[(6-amino-8-cyclopentyl-7,8-dihydro-7-oxopyrido[2,3-d]pyrimidin-2-yl)amino]-3-pyridinyl]-L-proline;
6-amino-8-cyclopentyl-2-[[5-[[4-(diethylamino)butyl]amino]-2-pyridinyl]amino]-pyrido[2,3-d]pyrimidin-7(8H)-one;
6-bromo-8-cyclopentyl-2-[[5-(1-piperidinyl)-2-pyridinyl]amino]-pyrido[2,3-d]pyrimidin-7(8H)-one;
6-bromo-8-cyclopentyl-2-[[5-(4-morpholinyl)-2-pyridinyl]amino]-pyrido[2,3-d]pyrimidin-7(8H)-one;
6-bromo-8-cyclopentyl-2-[[5-(diethylamino)-2-pyridinyl]amino]-pyrido[2,3-d]pyrimidin-7(8H)-one;
2-[[5-[bis(2-hydroxyethyl)amino]-2-pyridinyl]amino]-6-bromo-8-cyclopentyl-pyrido[2,3-d]pyrimidin-7(8H)-one;
2-[[5-[bis(2-methoxyethyl)amino]-2-pyridinyl]amino]-6-bromo-8-cyclopentyl-pyrido[2,3-d]pyrimidin-7(8H)-one;
2-[[5-[(2-aminoethyl)amino]-2-pyridinyl]amino]-6-bromo-8-cyclopentyl-pyrido[2,3-d]pyrimidin-7(8H)-one;
6-bromo-8-cyclopentyl-2-[[5-(dimethylamino)-2-pyridinyl]amino]-pyrido[2,3-d]pyrimidin-7(8H)-one;
N-[6-[(6-bromo-8-cyclopentyl-7,8-dihydro-7-oxopyrido[2,3-d]pyrimidin-2-yl)amino]-3-pyridinyl]-N-methyl-acetamide;
6-bromo-8-cyclopentyl-2-[[5-(2-methoxyethoxy)-2-pyridinyl]amino]-pyrido[2,3-d]pyrimidin-7(8H)-one;
6-bromo-8-cyclopentyl-2-[[5-[(2-methoxyethoxy)methyl]-2-pyridinyl]amino]-pyrido[2,3-d]pyrimidin-7(8H)-one;
6-bromo-8-cyclopentyl-2-[[5-[2-(diethylamino)ethoxy]-2-pyridinyl]amino]-pyrido[2,3-d]pyrimidin-7(8H)-one;
6-bromo-8-cyclopentyl-2-[[6-methyl-5-(1-piperazinyl)-2-pyridinyl]amino]-pyrido[2,3-d]pyrimidin-7(8H)-one;
6-bromo-8-cyclopentyl-2-[[5-(diethylamino)-2-pyridinyl]amino]-5-methyl-pyrido[2,3-d]pyrimidin-7(8H)-one;
2-[[5-[bis(2-hydroxyethyl)amino]-2-pyridinyl]amino]-6-bromo-8-cyclopentyl-5-methyl-pyrido[2,3-d]pyrimidin-7(8H)-one;
2-[[5-[(2-aminoethyl)amino]-2-pyridinyl]amino]-6-bromo-8-cyclopentyl-5-methyl-pyrido[2,3-d]pyrimidin-7(8H)-one;
6-bromo-8-cyclopentyl-2-[[5-(dimethylamino)-2-pyridinyl]amino]-5-methyl-pyrido[2,3-d]pyrimidin-7(8H)-one;
N-[6-[(6-bromo-8-cyclopentyl-7,8-dihydro-5-methyl-7-oxopyrido[2,3-d]pyrimidin-2-yl)amino]-3-pyridinyl]-N-methyl-acetamide;
6-bromo-8-cyclopentyl-2-[[5-(2-methoxyethoxy)-2-pyridinyl]amino]-5-methyl-pyrido[2,3-d]pyrimidin-7(8H)-one;
6-bromo-8-cyclopentyl-2-[[5-[(2-methoxyethoxy)methyl]-2-pyridinyl]amino]-5-methyl-pyrido[2,3-d]pyrimidin-7(8H)-one;
6-bromo-8-cyclopentyl-2-[[5-[2-(diethylamino)ethoxy]-2-pyridinyl]amino]-5-methyl-pyrido[2,3-d]pyrimidin-7(8H)-one;
6-bromo-8-cyclopentyl-5-methyl-2-[[5-(1-pyrrolidinyl)-2-pyridinyl]amino]-pyrido[2,3-d]pyrimidin-7(8H)-one;
6-bromo-8-cyclopentyl-5-methyl-2-[[6-methyl-5-(1-piperazinyl)-2-pyridinyl]amino]-pyrido[2,3-d]pyrimidin-7(8H)-one;
6-acetyl-8-cyclopentyl-2-[[5-(diethylamino)-2-pyridinyl]amino]-5-methyl-pyrido[2,3-d]pyrimidin-7(8H)-one;
6-acetyl-2-[[5-[bis(2-hydroxyethyl)amino]-2-pyridinyl]amino]-8-cyclopentyl-5-methyl-pyrido[2,3-d]pyrimidin-7(8H)-one;
6-acetyl-2-[[5-[(2-aminoethyl)amino]-2-pyridinyl]amino]-8-cyclopentyl-5-methyl-pyrido[2,3-d]pyrimidin-7(8H)-one;
6-acetyl-8-cyclopentyl-2-[[5-(dimethylamino)-2-pyridinyl]amino]-5-methyl-pyrido[2,3-d]pyrimidin-7(8H)-one;
N-[6-[(6-acetyl-8-cyclopentyl-7, 8-dihydro-5-methyl-7-oxopyrido[2,3-d]pyrimidin-2-yl)amino]-3-pyridinyl]-N-methyl-acetamide;
6-acetyl-8-cyclopentyl-2-[[5-(2-methoxyethoxy)-2-pyridinyl]amino]-5-methyl-pyrido[2,3-d]pyrimidin-7(8H)-one;
6-acetyl-8-cyclopentyl-2-[[5-[(2-methoxyethoxy)methyl]-2-pyridinyl]amino]-5-methyl-pyrido[2,3-d]pyrimidin-7(8H)-one;
6-acetyl-8-cyclopentyl-2-[[5-[2-(diethylamino)ethoxy]-2-pyridinyl]amino]-5-methyl-pyrido[2,3-d]pyrimidin-7(8H)-one;
6-acetyl-8-cyclopentyl-5-methyl-2-[[6-methyl-5-(1-piperazinyl)-2-pyridinyl]amino]-pyrido[2,3-d]pyrimidin-7(8H)-one;
6-acetyl-8-cyclopentyl-2-[[5-(1-piperidinyl)-2-pyridinyl]amino]-pyrido[2,3-d]pyrimidin-7(8H)-one;
6-acetyl-8-cyclopentyl-2-[[5-(4-morpholinyl)-2-pyridinyl]amino]-pyrido[2,3-d]pyrimidin-7(8H)-one;
6-acetyl-8-cyclopentyl-2-[[5-(diethylamino)-2-pyridinyl]amino]-pyrido[2,3-d]pyrimidin-7(8H)-one;
6-acetyl-2-[[5-[bis(2-hydroxyethyl)amino]-2-pyridinyl]amino]-8-cyclopentyl-pyrido[2,3-d]pyrimidin-7(8H)-one;
6-acetyl-2-[[5-[bis(2-methoxyethyl)amino]-2-pyridinyl]amino]-8-cyclopentyl-pyrido[2,3-d]pyrimidin-7(8H)-one;
6-acetyl-2-[[5-[(2-aminoethyl)amino]-2-pyridinyl]amino]-8-cyclopentyl-pyrido[2,3-d]pyrimidin-7(8H)-one;
6-acetyl-8-cyclopentyl-2-[[5-(dimethylamino)-2-pyridinyl]amino]-pyrido[2,3-d]pyrimidin-7(8H)-one;
N-[6-[(6-acetyl-8-cyclopentyl-7,8-dihydro-7-oxopyrido[2,3-d]pyrimidin-2-yl)amino]-3-pyridinyl]-N-methyl-acetamide;
6-acetyl-8-cyclopentyl-2-[[5-(2-methoxyethoxy)-2-pyridinyl]amino]-pyrido[2,3-d]pyrimidin-7(8H)-one;
6-acetyl-8-cyclopentyl-2-[[5-[(2-methoxyethoxy)methyl]-2-pyridinyl]amino]-pyrido[2,3-d]pyrimidin-7(8H)-one;
6-acetyl-8-cyclopentyl-2-[[5-[2-(diethylamino)ethoxy]-2-pyridinyl]amino]-pyrido[2,3-d]pyrimidin-7(8H)-one;
6-acetyl-8-cyclopentyl-2-[[5-(1-pyrrolidinyl)-2-pyridinyl]amino]-pyrido[2,3-d]pyrimidin-7(8H)-one;
6-acetyl-8-cyclopentyl-2-[[6-methyl-5-(1-piperazinyl)-2-pyridinyl]amino]-pyrido[2,3-d]pyrimidin-7(8H)-one;
6-bromo-8-cyclopentyl-2-[[5-[(2-methoxyethyl)amino]-2-pyridinyl]amino]-pyrido[2,3-d]pyrimidin-7(8H)-one;
2-[[5-(1-azetidinyl)-2-pyridinyl]amino]-6-bromo-8-cyclopentyl-pyrido[2,3-d]pyrimidin-7(8H)-one;
6-bromo-8-cyclopentyl-2-[[5-(hexahydro-1H-azepin-1-yl)-2-pyridinyl]amino]-pyrido[2,3-d]pyrimidin-7(8H)-one;
N-[6-[(6-bromo-8-cyclopentyl-7,8-dihydro-7-oxopyrido[2,3-d]pyrimidin-2-yl)amino]-3-pyridinyl]-acetamide;
6-bromo-8-cyclopentyl-2-[[5-(phenylamino)-2-pyridinyl]amino]-pyrido[2,3-d]pyrimidin-7(8H)-one;
6-bromo-8-cyclopentyl-2-[[5-[[(4-fluorophenyl)methyl]amino]-2-pyridinyl]amino]-pyrido[2,3-d]pyrimidin-7(8H)-one;
N-[6-[(6-bromo-8-cyclopentyl-7,8-dihydro-7-oxopyrido[2,3-d]pyrimidin-2-yl)amino]-3-pyridinyl]-methanesulfonamide;
6-bromo-8-cyclopentyl-2-[[5-(methylsulfonyl)-2-pyridinyl]amino]-pyrido[2,3-d]pyrimidin-7(8H)-one;
6-bromo-8-cyclopentyl-2-[(5-phenyl-2-pyridinyl)amino]-pyrido[2,3-d]pyrimidin-7(8H)-one;
6-amino-8-cyclopentyl-2-[[5-(2-methoxyethoxy)-2-pyridinyl]amino]-pyrido[2,3-d]pyrimidin-7(8H)-one;
6-amino-8-cyclopentyl-2-[[5-[(2-methoxyethyl)amino]-2-pyridinyl]amino]-pyrido[2,3-d]pyrimidin-7(8H)-one;
6-amino-2-[[5-(1-azetidinyl)-2-pyridinyl]amino]-8-cyclopentyl-pyrido[2,3-d]pyrimidin-7(8H)-one;
6-amino-8-cyclopentyl-2-[[5-(hexahydro-1H-azepin-1-yl)-2-pyridinyl]amino]-pyrido[2,3-d]pyrimidin-7(8H)-one;
N-[6-[(6-amino-8-cyclopentyl-7,8-dihydro-7-oxopyrido[2,3-d]pyrimidin-2-yl)amino]-3-pyridinyl]-acetamide;
6-amino-8-cyclopentyl-2-[[5-(phenylamino)-2-pyridinyl]amino]-pyrido[2,3-d]pyrimidin-7(8H)-one;
6-amino-8-cyclopentyl-2-[[5-[[(4-fluorophenyl)methyl]amino]-2-pyridinyl]amino]-pyrido[2,3-d]pyrimidin-7(8H)-one;
N-[6-[(6-amino-8-cyclopentyl-7,8-dihydro-7-oxopyrido[2,3-d]pyrimidin-2-yl)amino]-3-pyridinyl]-methanesulfonamide;
6-amino-8-cyclopentyl-2-[[5-(methylsulfonyl)-2-pyridinyl]amino]-pyrido[2,3-d]pyrimidin-7(8H)-one;
6-amino-8-cyclopentyl-2-[(5-phenyl-2-pyridinyl)amino]-pyrido[2,3-d]pyrimidin-7(8H)-one;
6-acetyl-8-cyclopentyl-2-[[5-[(2-methoxyethyl)amino]-2-pyridinyl]amino]-5-methyl-pyrido[2,3-d]pyrimidin-7(8H)-one;
6-acetyl-2-[[5-(1-azetidinyl)-2-pyridinyl]amino]-8-cyclopentyl-5-methyl-pyrido[2,3-d]pyrimidin-7(8H)-one;
6-acetyl-8-cyclopentyl-2-[[5-(hexahydro-1H-azepin-1-yl)-2-pyridinyl]amino]-5-methyl-pyrido[2,3-d]pyrimidin-7(8H)-one;
N-[6-[(6-acetyl-8-cyclopentyl-7, 8-dihydro-5-methyl-7-oxopyrido[2,3-d]pyrimidin-2-yl)amino]-3-pyridinyl]-acetamide;
6-acetyl-8-cyclopentyl-5-methyl-2-[[5-(phenylamino)-2-pyridinyl]amino]-pyrido[2,3-d]pyrimidin-7(8H)-one;
6-acetyl-8-cyclopentyl-2-[[5-[[(4-fluorophenyl)methyl]amino]-2-pyridinyl]amino]-5-methyl-pyrido[2,3-d]pyrimidin-7(8H)-one;
N-[6-[(6-acetyl-8-cyclopentyl-7,8-dihydro-5-methyl-7-oxopyrido[2,3-d]pyrimidin-2-yl)amino]-3-pyridinyl]-methanesulfonamide;
6-acetyl-8-cyclopentyl-5-methyl-2-[[5-(methylsulfonyl)-2-pyridinyl]amino]-pyrido[2,3-d]pyrimidin-7(8H)-one;
6-acetyl-8-cyclopentyl-5-methyl-2-[(5-phenyl-2-pyridinyl)amino]-pyrido[2,3-d]pyrimidin-7(8H)-one;
8-cyclopentyl-2-[[5-(2-methoxyethoxy)-2-pyridinyl]amino]-6-(phenylmethyl)-pyrido[2,3-d]pyrimidin-7(8H)-one;
8-cyclopentyl-2-[[5-[(2-methoxyethyl)amino]-2-pyridinyl]amino]-6-(phenylmethyl)-pyrido[2,3-d]pyrimidin-7(8H)-one;
2-[[5-(1-azetidinyl)-2-pyridinyl]amino]-8-cyclopentyl-6-(phenylmethyl)-pyrido[2,3-d]pyrimidin-7(8H)-one;
8-cyclopentyl-2-[[5-(hexahydro-1H-azepin-1-yl)-2-pyridinyl]amino]-6-(phenylmethyl)-pyrido[2,3-d]pyrimidin-7(8H)-one;
N-[6-[[8-cyclopentyl-7,8-dihydro-7-oxo-6-(phenylmethyl)pyrido[2,3-d]pyrimidin-2-yl]amino]-3-pyridinyl]-acetamide;
8-cyclopentyl-2-[[5-(phenylamino)-2-pyridinyl]amino]-6-(phenylmethyl)-pyrido[2,3-d]pyrimidin-7(8H)-one;
8-cyclopentyl-2-[[5-[[(4-fluorophenyl)methyl]amino]-2-pyridinyl]amino]-6-(phenylmethyl)-pyrido[2,3-d]pyrimidin-7(8H)-one;
N-[6-[[8-cyclopentyl-7,8-dihydro-7-oxo-6-(phenylmethyl)pyrido[2,3-d]pyrimidin-2-yl]amino]-3-pyridinyl]-methanesulfonamide;
8-cyclopentyl-2-[[5-(methylsulfonyl)-2-pyridinyl]amino]-6-(phenylmethyl)-pyrido[2,3-d]pyrimidin-7(8H)-one;
8-cyclopentyl-6-(phenylmethyl)-2-[(5-phenyl-2-pyridinyl)amino]-pyrido[2,3-d]pyrimidin-7(8H)-one;
8-cyclopentyl-6-(hydroxymethyl)-2-[[5-(2-methoxyethoxy)-2-pyridinyl]amino]-pyrido[2,3-d]pyrimidin-7(8H)-one;
8-cyclopentyl-6-(hydroxymethyl)-2-[[5-[(2-methoxyethyl)amino]-2-pyridinyl]amino]-pyrido[2,3-d]pyrimidin-7(8H)-one;
2-[[5-(1-azetidinyl)-2-pyridinyl]amino]-8-cyclopentyl-6-(hydroxymethyl)-pyrido[2,3-d]pyrimidin-7(8H)-one;
8-cyclopentyl-2-[[5-(hexahydro-1H-azepin-1-yl)-2-pyridinyl]amino]-6-(hydroxymethyl)-pyrido[2,3-d]pyrimidin-7(8H)-one;
N-[6-[[8-cyclopentyl-7,8-dihydro-6-(hydroxymethyl)-7-oxopyrido[2,3-d]pyrimidin-2-yl]amino]-3-pyridinyl]-acetamide;
8-cyclopentyl-6-(hydroxymethyl)-2-[[5-(phenylamino)-2-pyridinyl]amino]-pyrido[2,3-d]pyrimidin-7(8H)-one;
8-cyclopentyl-2-[[5-[[(4-fluorophenyl)methyl]amino]-2-pyridinyl]amino]-6-(hydroxymethyl)-pyrido[2,3-d]pyrimidin-7(8H)-one;
N-[6-[[8-cyclopentyl-7,8-dihydro-6-(hydroxymethyl)-7-oxopyrido[2,3-d]pyrimidin-2-yl]amino]-3-pyridinyl]-methanesulfonamide;
8-cyclopentyl-6-(hydroxymethyl)-2-[[5-(methylsulfonyl)-2-pyridinyl]amino]-pyrido[2,3-d]pyrimidin-7(8H)-one;
8-cyclopentyl-6-(hydroxymethyl)-2-[(5-phenyl-2-pyridinyl)amino]-pyrido[2,3-d]pyrimidin-7(8H)-one;
8-cyclopentyl-6-ethyl-2-[[5-(2-methoxyethoxy)-2-pyridinyl]amino]-pyrido[2,3-d]pyrimidin-7(8H)-one;
8-cyclopentyl-6-ethyl-2-[[5-[(2-methoxyethyl)amino]-2-pyridinyl]amino]-pyrido[2,3-d]pyrimidin-7(8H)-one;
2-[[5-(1-azetidinyl)-2-pyridinyl]amino]-8-cyclopentyl-6-ethyl-pyrido[2,3-d]pyrimidin-7(8H)-one;
8-cyclopentyl-6-ethyl-2-[[5-(hexahydro-1H-azepin-1-yl)-2-pyridinyl]amino]-pyrido[2,3-d]pyrimidin-7(8H)-one;
N-[6-[(8-cyclopentyl-6-ethyl-7,8-dihydro-7-oxopyrido[2,3-d]pyrimidin-2-yl)amino]-3-pyridinyl]-acetamide;
8-cyclopentyl-6-ethyl-2-[[5-(phenylamino)-2-pyridinyl]amino]-pyrido[2,3-d]pyrimidin-7(8H)-one;
8-cyclopentyl-6-ethyl-2-[[5-[[(4-fluorophenyl)methyl]amino]-2-pyridinyl]amino]-pyrido[2,3-d]pyrimidin-7(8H)-one;
N-[6-[(8-cyclopentyl-6-ethyl-7,8-dihydro-7-oxopyrido[2,3-d]pyrimidin-2-yl)amino]-3-pyridinyl]-methanesulfonamide;
8-cyclopentyl-6-ethyl-2-[[5-(methylsulfonyl)-2-pyridinyl]amino]-pyrido[2,3-d]pyrimidin-7(8H)-one;
8-cyclopentyl-6-ethyl-2-[(5-phenyl-2-pyridinyl)amino]-pyrido[2,3-d]pyrimidin-7(8H)-one;
6-bromo-8-cyclopentyl-2-[[5-(1-piperazinylcarbonyl)-2-pyridinyl]amino]-pyrido[2,3-d]pyrimidin-7(8H)-one;
6-bromo-8-cyclopentyl-2-[[5-[(3,5-dimethyl-1-piperazinyl)carbonyl]-2-pyridinyl]amino]-pyrido[2,3-d]pyrimidin-7(8H)-one;
2-[[5-[(3-amino-1-pyrrolidinyl)carbonyl]-2-pyridinyl]amino]-6-bromo-8-cyclopentyl-pyrido[2,3-d]pyrimidin-7(8H)-one;
6-bromo-8-cyclopentyl-2-[[5-(4-morpholinylcarbonyl)-2-pyridinyl]amino]-pyrido[2,3-d]pyrimidin-7(8H)-one;
6-bromo-8-cyclopentyl-5-methyl-2-[[5-(1-piperazinylcarbonyl)-2-pyridinyl]amino]-pyrido[2,3-d]pyrimidin-7(8H)-one;
6-bromo-8-cyclopentyl-2-[[5-[(3,5-dimethyl-1-piperazinyl)carbonyl]-2-pyridinyl]amino]-5-methyl-pyrido[2,3-d]pyrimidin-7(8H)-one;
2-[[5-[(3-amino-1-pyrrolidinyl)carbonyl]-2-pyridinyl]amino]-6-bromo-8-cyclopentyl-5-methyl-pyrido[2,3-d]pyrimidin-7(8H)-one;
6-bromo-8-cyclopentyl-5-methyl-2-[[5-(4-morpholinylcarbonyl)-2-pyridinyl]amino]-pyrido[2,3-d]pyrimidin-7(8H)-one;
6-acetyl-8-cyclopentyl-5-methyl-2-[[5-(1-piperazinylcarbonyl)-2-pyridinyl]amino]-pyrido[2,3-d]pyrimidin-7(8H)-one;
6-acetyl-8-cyclopentyl-2-[[5-[(3,5-dimethyl-1-piperazinyl)carbonyl]-2-pyridinyl]amino]-5-methyl-pyrido[2,3-d]pyrimidin-7(8H)-one;
6-acetyl-2-[[5-[(3-amino-1-pyrrolidinyl)carbonyl]-2-pyridinyl]amino]-8-cyclopentyl-5-methyl-pyrido[2,3-d]pyrimidin-7(8H)-one;
6-acetyl-8-cyclopentyl-5-methyl-2-[[5-(4-morpholinylcarbonyl)-2-pyridinyl]amino]-pyrido[2,3-d]pyrimidin-7(8H)-one;
8-cyclopentyl-6-ethyl-2-[[5-(1-piperazinylcarbonyl)-2-pyridinyl]amino]-pyrido[2,3-d]pyrimidin-7(8H)-one;
8-cyclopentyl-2-[[5-[(3,5-dimethyl-1-piperazinyl)carbonyl]-2-pyridinyl]amino]-6-ethyl-pyrido[2,3-d]pyrimidin-7(8H)-one;
2-[[5-[(3-amino-1-pyrrolidinyl)carbonyl]-2-pyridinyl]amino]-8-cyclopentyl-6-ethyl-pyrido[2,3-d]pyrimidin-7(8H)-one;
8-cyclopentyl-6-ethyl-2-[[5-(4-morpholinylcarbonyl)-2-pyridinyl]amino]-pyrido[2,3-d]pyrimidin-7(8H)-one;
6-bromo-8-cyclopentyl-2-[[5-(1-piperazinylsulfonyl)-2-pyridinyl]amino]-pyrido[2,3-d]pyrimidin-7(8H)-one;
6-bromo-8-cyclopentyl-2-[[5-(4-morpholinylsulfonyl)-2-pyridinyl]amino]-pyrido[2,3-d]pyrimidin-7(8H)-one;
2-[[5-[(3-amino-1-pyrrolidinyl)sulfonyl]-2-pyridinyl]amino]-6-bromo-8-cyclopentyl-pyrido[2,3-d]pyrimidin-7(8H)-one;
6-bromo-8-cyclopentyl-2-[[5-[(3,5-dimethyl-1-piperazinyl)sulfonyl]-2-pyridinyl]amino]-pyrido[2,3-d]pyrimidin-7(8H)-one;
6-bromo-8-cyclopentyl-5-methyl-2-[[5-(1-piperazinylsulfonyl)-2-pyridinyl]amino]-pyrido[2,3-d]pyrimidin-7(8H)-one;
6-bromo-8-cyclopentyl-5-methyl-2-[[5-(4-morpholinylsulfonyl)-2-pyridinyl]amino]-pyrido[2,3-d]pyrimidin-7(8H)-one;
2-[[5-[(3-amino-1-pyrrolidinyl)sulfonyl]-2-pyridinyl]amino]-6-bromo-8-cyclopentyl-5-methyl-pyrido[2,3-d]pyrimidin-7(8H)-one;
6-bromo-8-cyclopentyl-2-[[5-[(3,5-dimethyl-1-piperazinyl)sulfonyl]-2-pyridinyl]amino]-5-methyl-pyrido[2,3-d]pyrimidin-7(8H)-one;
8-cyclopentyl-6-ethyl-2-[[5-(1-piperazinylsulfonyl)-2-pyridinyl]amino]-pyrido[2,3-d]pyrimidin-7(8H)-one;
8-cyclopentyl-6-ethyl-2-[[5-(4-morpholinylsulfonyl)-2-pyridinyl]amino]-pyrido[2,3-d]pyrimidin-7(8H)-one;
2-[[5-[(3-amino-1-pyrrolidinyl)sulfonyl]-2-pyridinyl]amino]-8-cyclopentyl-6-ethyl-pyrido[2,3-d]pyrimidin-7(8H)-one;
8-cyclopentyl-2-[[5-[(3,5-dimethyl-1-piperazinyl)sulfonyl]-2-pyridinyl]amino]-6-ethyl-pyrido[2,3-d]pyrimidin-7(8H)-one;
6-acetyl-8-cyclopentyl-5-methyl-2-[[5-(1-piperazinylsulfonyl)-2-pyridinyl]amino]-pyrido[2,3-d]pyrimidin-7(8H)-one;
6-acetyl-8-cyclopentyl-5-methyl-2-[[5-(4-morpholinylsulfonyl)-2-pyridinyl]amino]-pyrido[2,3-d]pyrimidin-7(8H)-one;
6-acetyl-2-[[5-[(3-amino-1-pyrrolidinyl)sulfonyl]-2-pyridinyl]amino]-8-cyclopentyl-5-methyl-pyrido[2,3-d]pyrimidin-7(8H)-one;
6-acetyl-8-cyclopentyl-2-[[5-[(3,5-dimethyl-1-piperazinyl)sulfonyl]-2-pyridinyl]amino]-5-methyl-pyrido[2,3-d]pyrimidin-7(8H)-one;
6-acetyl-8-cyclopentyl-5-methyl-2-(1,6-naphthyridin-2-ylamino)-pyrido[2,3-d]pyrimidin-7(8H)-one;
6-acetyl-8-cyclopentyl-2-[[5-(1, 1-dioxido-4-thiomorpholinyl)-2-pyridinyl]amino]-5-methyl-pyrido[2,3-d]pyrimidin-7(8H)-one;
8-cyclopentyl-6-(hydroxymethyl)-5-methyl-2-[[5-(1-piperazinyl)-2-pyridinyl]amino]-pyrido[2,3-d]pyrimidin-7(8H)-one;
6-acetyl-2-[[3-chloro-5-(1-piperazinyl)-2-pyridinyl]amino]-8-cyclopentyl-5-methyl-pyrido[2,3-d]pyrimidin-7(8H)-one;
4-[6-acetyl-5-methyl-7-oxo-2-(2-pyridinylamino)pyrido[2,3-d]pyrimidin-8(7H)-yl]-cyclohexanecarboxylic acid;
4-[6-acetyl-2-[[5-(dimethylamino)-2-pyridinyl]amino]-5-methyl-7-oxopyrido[2,3-d]pyrimidin-8(7H)-yl]-cyclohexanecarboxylic acid;
6-[(8-cyclopentyl-6-ethyl-7,8-dihydro-7-oxopyrido[2,3-d]pyrimidin-2-yl)amino]-3-(1-piperazinyl)-2-pyridinecarboxylic acid;
2-[[6-acetyl-5-(1-piperazinyl)-2-pyridinyl]amino]-8-cyclopentyl-6-ethyl-pyrido[2,3-d]pyrimidin-7(8H)-one;
3-[2-[[6-[(8-cyclopentyl-6-ethyl-7,8-dihydro-7-oxopyrido[2,3-d]pyrimidin-2-yl)amino]-3-pyridinyl]oxy]ethoxy]-propanoic acid;
2-[[6-[(8-cyclopentyl-6-ethyl-7,8-dihydro-7-oxopyrido[2,3-d]pyrimidin-2-yl)amino]-3-pyridinyl]oxy]-acetic acid;
8-cyclopentyl-2-[[5-[2-[2-(5-methyl-2-pyridinyl)ethoxy]ethoxy]-2-pyridinyl]amino]-pyrido[2,3-d]pyrimidin-7(8H)-one;
8-cyclopentyl-6-ethyl-2-[[5-[2-(2-methoxyethoxy)ethoxy]-2-pyridinyl]amino]-pyrido[2,3-d]pyrimidin-7(8H)-one;
8-cyclopentyl-2-[[5-[[[3-(1-piperazinyl)propyl]amino]methyl]-2-pyridinyl]amino]-pyrido[2,3-d]pyrimidin-7(8H)-one;
8-cyclopentyl-2-[[5-[[[3-(1H-imidazol-1-yl)propyl]amino]methyl]-2-pyridinyl]amino]-pyrido[2,3-d]pyrimidin-7(8H)-one;
6-acetyl-5-methyl-2-[(5-methyl-2-pyridinyl)amino]-8-(4-piperidinyl)-pyrido[2,3-d]pyrimidin-7(8H)-one;
6-acetyl-2-[[5-(3,4-dihydroxy-1-pyrrolidinyl)-2-pyridinyl]amino]-8-(methoxymethyl)-5-methyl-pyrido[2,3-d]pyrimidin-7(8H)-one;
6-bromo-8-cyclopentyl-2-[[(4-methoxyphenyl)methyl]amino]-5-methyl-pyrido[2,3-d]pyrimidin-7(8H)-one;
8-cyclopentyl-6-(1-ethoxyethenyl)-2-[[(4-methoxyphenyl)methyl]amino]-5-methyl-pyrido[2,3-d]pyrimidin-7(8H)-one; and
8-cyclopentyl-6-ethyl-2-[[5-[2-(phenylsulfonyl)ethoxy]-2-pyridinyl]amino]-pyrido[2,3-d]pyrimidin-7(8H)-one.
   Other cell cycle inhibitors described in the following patents and patent applications can be used in this invention:
US20120244110 [AMGEN INC.];
US20110142796 [AMGEN INC.];
US20110097305 [AMGEN INC.];
US20100004243 [ASTEX THERAPEUTICS LTD];
US20090318430 [ASTEX THERAPEUTICS LTD];
US20100105653 [ASTEX THERAPEUTICS LTD];
U.S. Pat. No. 7,470,709 [Novartis];
U.S. Pat. No. 7,378,423 [Japan Tobacco];
U.S. Pat. No. 7,446,195 [Schering];
U.S. Pat. No. 8,163,762 [CNRS];
U.S. Pat. No. 8,067,424 [Emery University];
U.S. Pat. No. 6,479,487 [Aventis];
U.S. Pat. No. 6,413,974 [Aventis];
U.S. Pat. No. 6,596,694 [Aventis];
U.S. Pat. No. 6,642,231 [Aventis];
U.S. Pat. No. 7,429,595 [Aventis];
U.S. Pat. No. 8,273,754 [Arqule];
U.S. Pat. No. 6,620,831 [LG Lifesciences]
U.S. Pat. No. 7,271,193 [NICHOLAS PIRAMAL INDIA LTD];
U.S. Pat. No. 7,884,127 [NICHOLAS PIRAMAL INDIA LTD];
U.S. Pat. No. 7,915,301 [NICHOLAS PIRAMAL INDIA LTD];
U.S. Pat. No. 7,456,168 [Warner-Lambert];
U.S. Pat. No. 7,053,070 [Warner-Lambert];
U.S. Pat. No. 6,498,163 [Warner-Lambert];
U.S. Pat. No. 7,713,994 [Wyeth];
US201103012161 [THE UAB RESEARCH FOUNDATION];
US20110152244 [NOVARTIS AG];
US20100272678 [KASINA LAILA INNOVA PHARMACEUTICALS PRIVATE LTD];
US20100179210 [NICHOLAS PIRAMAL INDIA LTD];
US20090326229 [Takeda Pharmaceutical Company Ltd];
US20090093507 [Curis Inc.]; and
U.S. Pat. No. 6,630,464 [The Government of the United States of America];
   which are herein incorporated by reference in their entirety, particularly in parts disclosing cell cycle inhibitors.

### Proteins of Interest

The methods of the invention can be used to culture cells that express recombinant proteins of interest. The expressed recombinant proteins may be secreted into the culture medium from which they can be recovered and/or collected. In addition, the proteins can be purified, or partially purified, from such culture or component (e.g., from culture medium) using known processes and products available from commercial vendors. The purified proteins can then be “formulated”, meaning buffer exchanged, sterilized, bulk-packaged, and/or packaged for a final user. Suitable formulations for pharmaceutical compositions include those described in Remington's Pharmaceutical Sciences, 18^{th} ed. 1995, Mack Publishing Company, Easton, Pa.

Examples of polypeptides that can be produced with the methods of the invention include proteins comprising amino acid sequences identical to or substantially similar to all or part of one of the following proteins: tumor necrosis factor (TNF), flt3 ligand (WO 94/28391), thrombopoeitin, calcitonin, IL-2, angiopoietin-2 (Maisonpierre et al. (1997), Science 277(5322): 55-60), ligand for receptor activator of NF-kappa B (RANKL, WO 01/36637), tumor necrosis factor (TNF)-related apoptosis-inducing ligand (TRAIL, WO 97/01633), thymic stroma-derived lymnphopoietin, granulocyte colony stimulating factor, granulocyte-macrophage colony stimulating factor (GM-CSF, Australian Patent No. 588819), mast cell growth factor, stem cell growth factor (U.S. Pat. No. 6,204,363), epidermal growth factor, keratinocyte growth factor, megakaryote growth and development factor, RANTES, human fibrinogen-like 2 protein (FGL2; NCBI accession no. NM_00682; Riegg and Pytela (1995), Gene 160:257-62) growth hormone, insulin, insulinotropin, insulin-like growth factors, parathyroid hormone, interferons including α-interferons, γ-interferon, and consensus interferons (U.S. Pat. Nos. 4,695,623 and 4,897,471), nerve growth factor, brain-derived neurotrophic factor, synaptotagmin-like proteins (SLP 1-5), neurotrophin-3, glucagon, interleukins, colony stimulating factors, lymphotoxin-β, leukemia inhibitory factor, and oncostatin-M. Descriptions of proteins that can be produced according to the inventive methods may be found in, for example, Human Cytokines: Handbook for Basic and Clinical Researchz, all volumes (Aggarwal and Gutterman, eds. Blackwell Sciences, Cambridge, Mass., 1998); Growth Factors: A Practical Approach (McKay and Leigh, eds., Oxford University Press Inc., New York, 1993); and The Cytokine Handbook, Vols. 1 and 2 (Thompson and Lotze eds., Academic Press, San Diego, Calif., 2003).

Additionally the methods of the invention would be useful to produce proteins comprising all or part of the amino acid sequence of a receptor for any of the above-mentioned proteins, an antagonist to such a receptor or any of the above-mentioned proteins, and/or proteins substantially similar to such receptors or antagonists. These receptors and antagonists include: both forms of tumor necrosis factor receptor (TNFR, referred to as p55 and p75, U.S. Pat. No. 5,395,760 and U.S. Pat. No. 5,610,279), Interleukin-1 (IL-1) receptors (types I and II; EP Patent No. 0460846, U.S. Pat. No. 4,968,607, and U.S. Pat. No. 5,767,064), IL-1 receptor antagonists (U.S. Pat. No. 6,337,072), IL-1 antagonists or inhibitors (U.S. Pat. Nos. 5,981,713, 6,096,728, and 5,075,222) IL-2 receptors, IL-4 receptors (EP Patent No. 0 367 566 and U.S. Pat. No. 5,856,296), IL-15 receptors, IL-17 receptors, IL-18 receptors, Fc receptors, granulocyte-macrophage colony stimulating factor receptor, granulocyte colony stimulating factor receptor, receptors for oncostatin-M and leukemia inhibitory factor, receptor activator of NF-kappa B (RANK, WO 01/36637 and U.S. Pat. No. 6,271,349), osteoprotegerin (U.S. Pat. No. 6,015,938), receptors for TRAIL (including TRAIL receptors 1, 2, 3, and 4), and receptors that comprise death domains, such as Fas or Apoptosis-Inducing Receptor (AIR).

Other proteins that can be produced using the invention include proteins comprising all or part of the amino acid sequences of differentiation antigens (referred to as CD proteins) or their ligands or proteins substantially similar to either of these. Such antigens are disclosed in Leukocyte Typing VI (Proceedings of the Vith International Workshop and Conference, Kishimoto, Kikutani et al., eds., Kobe, Japan, 1996). Similar CD proteins are disclosed in subsequent workshops. Examples of such antigens include CD22, CD27, CD30, CD39, CD40, and ligands thereto (CD27 ligand, CD30 ligand, etc.). Several of the CD antigens are members of the TNF receptor family, which also includes 41BB and OX40. The ligands are often members of the TNF family, as are 41BB ligand and OX40 ligand.

Enzymatically active proteins or their ligands can also be produced using the invention.

Examples include proteins comprising all or part of one of the following proteins or their ligands or a protein substantially similar to one of these: a disintegrin and metalloproteinase domain family members including TNF-alpha Converting Enzyme, various kinases, glucocerebrosidase, superoxide dismutase, tissue plasminogen activator, Factor VIII, Factor IX, apolipoprotein E, apolipoprotein A-I, globins, an IL-2 antagonist, alpha-1 antitrypsin, ligands for any of the above-mentioned enzymes, and numerous other enzymes and their ligands.

Examples of antibodies that can be produced include, but are not limited to, those that recognize any one or a combination of proteins including, but not limited to, the above-mentioned proteins and/or the following antigens: CD2, CD3, CD4, CD8, CD11a, CD14, CD18, CD20, CD22, CD23, CD25, CD33, CD40, CD44, CD52, CD80 (B7.1), CD86 (B7.2), CD147, IL-1α, IL-1β, IL-2, IL-3, IL-7, IL-4, IL-5, IL-8, IL-10, IL-2 receptor, IL-4 receptor, IL-6 receptor, IL-13 receptor, IL-18 receptor subunits, FGL2, PDGF-β and analogs thereof (see U.S. Pat. Nos. 5,272,064 and 5,149,792), VEGF, TGF, TGF-β2, TGF-β1, EGF receptor (see U.S. Pat. No. 6,235,883) VEGF receptor, hepatocyte growth factor, osteoprotegerin ligand, interferon gamma, B lymphocyte stimulator (BlyS, also known as BAFF, THANK, TALL-1, and zTNF4; see Do and Chen-Kiang (2002), Cytokine Growth Factor Rev. 13(1): 19-25), C5 complement, IgE, tumor antigen CA125, tumor antigen MUC1, PEM antigen, LCG (which is a gene product that is expressed in association with lung cancer), HER-2, HER-3, a tumor-associated glycoprotein TAG-72, the SK-1 antigen, tumor-associated epitopes that are present in elevated levels in the sera of patients with colon and/or pancreatic cancer, cancer-associated epitopes or proteins expressed on breast, colon, squamous cell, prostate, pancreatic, lung, and/or kidney cancer cells and/or on melanoma, glioma, or neuroblastoma cells, the necrotic core of a tumor, integrin alpha 4 beta 7, the integrin VLA-4, integrins (including integrins comprising alpha4beta7), TRAIL receptors 1, 2, 3, and 4, RANK, RANK ligand, TNF-α, the adhesion molecule VAP-1, epithelial cell adhesion molecule (EpCAM), intercellular adhesion molecule-3 (ICAM-3), leukointegrin adhesin, the platelet glycoprotein gp Iib/IIIa, cardiac myosin heavy chain, parathyroid hormone, rNAPc2 (which is an inhibitor of factor VIIa-tissue factor), MHC I, carcinoembryonic antigen (CEA), alpha-fetoprotein (AFP), tumor necrosis factor (TNF), CTLA-4 (which is a cytotoxic T lymphocyte-associated antigen), Fc-γ-1 receptor, HLA-DR 10 beta, HLA-DR antigen, sclerostin, L-selectin, Respiratory Syncitial Virus, human immunodeficiency virus (HIV), hepatitis B virus (HBV), Streptococcus mutans, and Staphlycoccus aureus.

Specific examples of known antibodies which can be produced using the methods of the invention include but are not limited to adalimumab, bevacizumab, infliximab, abciximab, alemtuzurab, bapineuzumab, basiliximab, belimumab, briakinumab, brodalumab, canakinumab, certolizumab pegol, cetuximab, conatunmumab, denosumab, eculizumab, etrolizumab, gemtuzumab ozogamicin, golimumab, ibritumomab tiuxetan, labetuzumab, mapatumurmab, matuzumab, mepolizumab, motavizumab, muromonab-CD3, natalizumab, nimotuzumab, ofatumumab, omalizumab, oregovomab, palivizumab, panitumumab, pemtumomab, pertuzumnab, ranibizumab, rituximab, rovelizumab, tocilizumab, tositumomab, trastuzumab, ustekinumab, vedolizomab, zalutumumab, and zanolimumab.

The invention can also be used to produce recombinant fusion proteins comprising, for example, any of the above-mentioned proteins. For example, recombinant fusion proteins comprising one of the above-mentioned proteins plus a multimerization domain, such as a leucine zipper, a coiled coil, an Fc portion of an immunoglobulin, or a substantially similar protein, can be produced using the methods of the invention. See e.g. WO94/10308; Lovejoy et al. (1993), Science 259:1288-1293; Harbury et al. (1993), Science 262:1401-05; Harbury et al. (1994). Nature 371:80-83; Hikansson et al. (1999). Structure 7:255-64. Specifically included among such recombinant fusion proteins are proteins in which a portion of a receptor is fused to an Fc portion of an antibody such as etanercept (a p75 TNFR:Fc), abatacept and belatacept (CTLA4:Fc).

The present invention is not to be limited in scope by the specific embodiments described herein that are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

### EXAMPLES

### Example 1

Cell cycle inhibitors are identified using the following general approach. Initially, the cell cycle in mammalian cells, notably CHO cells, was examined. This analysis provided insight into various pathways involved in cell cycle, transcription, translation, and secretion of proteins, as well as glycosylation. Representative steps identified as points at which intervention may modulate the cell cycle and induce growth arrest include modulation of CDKs and their substrates and other endogenous proteins that affect these cell cycle regulators.

A list of candidate compounds was compiled which correlated with several points of intervention as described herein. Compounds are tested for their ability to control viable cell density, maintain cell viability, maintain product quality and also increase specific productivity (product per cell). In each case a cell culture expressing a recombinant human antibody (Antibody A) is incubated with one candidate cell cycle inhibitory compound, which is added at varying concentrations to cell culture media.

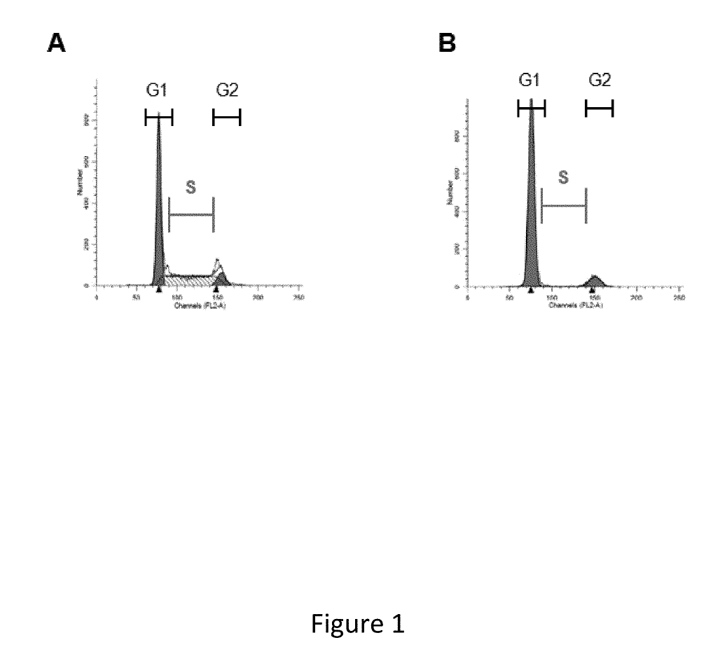

In one set of experiments, a cell cycle analysis was performed for various cell cycle inhibitory compounds. Briefly, CHO cells are grown in 24 deep well plate (for example, Corning Axygen deep well plates; Corning, Amsterdam, The Netherlands), using standard cell-culture medium, under conditions promoting growth of the cells (for example 36 degreesC, 5% CO2) in the presence of various cell cycle inhibitors. Samples are collected at various time points, and cell cycle profiles are obtained by propidium iodide (PI) staining followed by fluorescence-activated cell sorting (FACS) analysis. An example of the results obtained by FACS analysis is shown in FIG. 1. Results for compounds 6-acetyl-8-cyclopentyl-5-methyl-2-((5-(1-piperazinyl)-2-pyridinyl)amino)pyrido[2,3-d]pyridin-7(8H)-one (Compound A) and N-(6-(4-(dimethylamino)-1-piperidinyl)-3-pyridazinyl)-9-(trans-4-methylcyclohexyl)-9H-pyrido[4′,3′,:4,5]pyrrolo[2,3-d]pyrimidin-2-amine (Compound B) are shown in Table 1 below.

TABLE 1

| | | Cell Cycle Distribution (%) | | |
|---|---|---|---|---|
| | Day | G1 | S | G2/M |
| Control | 1 | 80.6 | 18.7 | 0.7 |
| | 2 | 78.8 | 18.7 | 2.5 |
| | 3 | 76.7 | 20.76 | 2.6 |
| | 4 | 81.5 | 15.5 | 3 |
| Compound A | 1 | 95.7 | 2.6 | 1.8 |
| (5 microM) | 2 | 96.7 | 2.1 | 1.2 |
| | 3 | 93.5 | 4.8 | 1.6 |
| | 4 | 94.8 | 5 | 0.26 |
| Compound B | 1 | 91.22 | 6.04 | 2.74 |
| (5 microM) | 2 | 74.82 | 19.43 | 5.75 |
| | 3 | 79.21 | 17.3 | 3.49 |
| | 4 | 78.68 | 15.19 | 6.13 |

Similar testing is performed with additional compounds; compounds that demonstrate increasing G1 arrest (i.e., sustained percentage of cells in G1 as compared to control, or decreased percentage of cells in S phase as compare to control) are evaluated in further experiments for their effects, if any, on the growth of recombinant cell lines expressing recombinant human monoclonal antibody and the specific productivity of the recombinant antibody, as described below.

### Example 2

Transfected CHO cells expressing a recombinant human monoclonal antibody (mAb) are cultured in 24-deep well plates with seeding density at 1×10⁷/mL for five days. At certain time periods, samples of the cultures are taken and evaluated for viable cell density (VCD; expressed in 10⁶ cells/mL); percent viability (as determined by trypan blue exclusion); titer of recombinant protein (expressed in grams of protein per liter of culture, or g/L, as determined by Protein A affinity HPLC); qP, or specific productivity (expressed in picograms of recombinant protein per cell per day, or pg/c/d), the presence or absence of high molecular weight substances (usually an indicator of protein aggregation; expressed as % of the total protein); and the relative percentage of Mannose 5, a major intermediate glyco-form that attaches to N297 of the mAb. N-linked glycosylation has an impact on the tertiary structure of the antibody and may affect Fc mediated antibody effector functions. The majority of the human Fc glycans are complex biantennary structures with galactose as the prevalent terminal sugar. Immature glyco-forms, such as high-mannose glycol-forms (Man5-9), are of concern due to potential higher plasma clearance rate (as compared to the clearance rate of complex glycan linked mAbs).

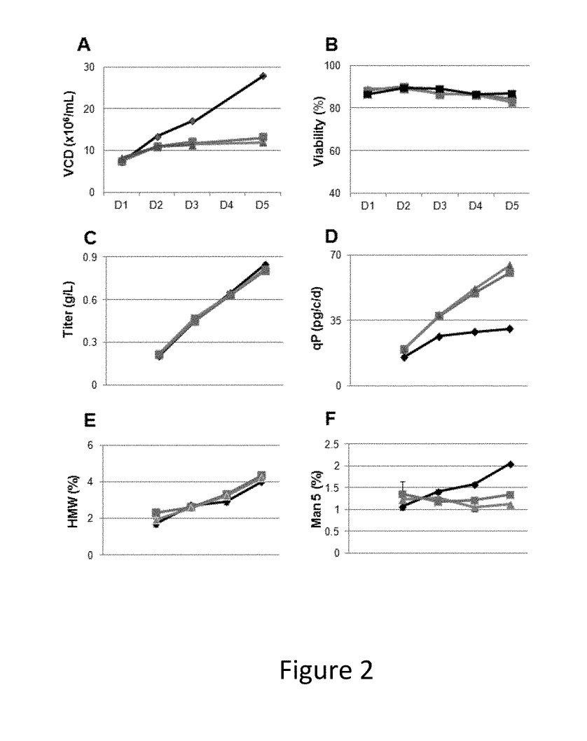

Results of certain compounds are shown in FIG. 2, which shows the observed effect of the cell cycle inhibitor tested on the VCD and viability of cells expressing Antibody A. It also shows the effects on productivity, such as titer and qP, as well as product quality attributes of % HMW and % Man5. As shown in FIG. 2, the tested compound provided a significant inhibition of proliferation without inducing cell death. In the meantime, qP was increased more than two fold. Additionally, no detrimental effects on quality attributes of the mAb (specifically high molecular weight (HMW) and high mannose (Man 5)), were observed. Compounds that demonstrated favorable (arrest cell growth and increase qP without significant impact on viability and product quality) were evaluated for stability in culture medium at room temperature for two weeks; compounds that demonstrated sufficient stability were further evaluated in a bioreactor production process as described in Example 3.

### Example 3

In another set of experiments, several cell cycle inhibitors are tested as a component of a cell culture expressing Antibody A in a bioreactor production process. Production bioreactors are set up and run substantially as described in US Patent application 20120214204, or by other methods or protocols known in the art. Briefly, the compounds are evaluated for their effects on cells expressing recombinant protein in 2 L bioreactors. The compound to be tested is added into the bioreactor at a predetermined concentration at day 8; samples are taken at various time points both before and after the addition of the compound, and evaluated for VCD, % viability, qP, titer of mAb produced percentage of protein that formed high molecular weight aggregates, and percentage of high mannose.

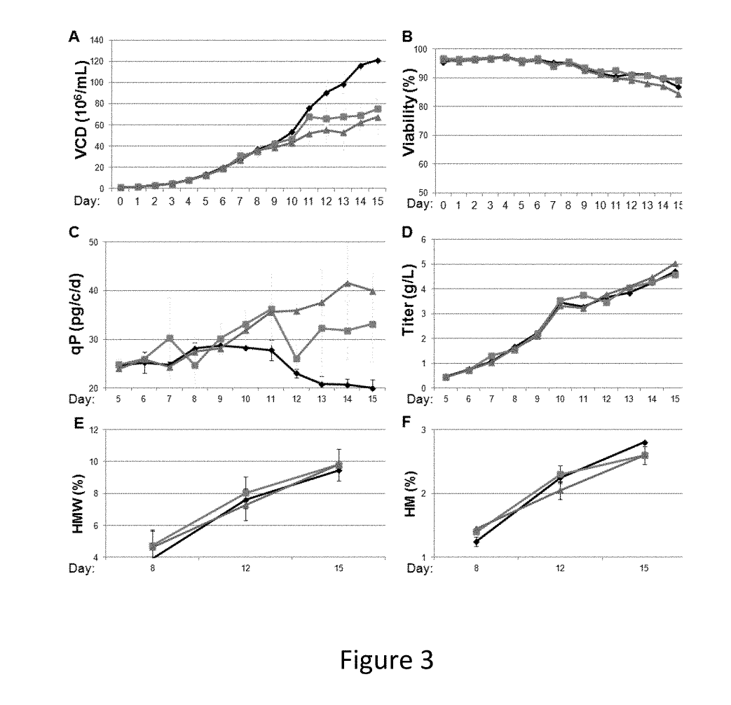

FIG. 3 shows the observed effect of cell cycle inhibition on VCD of cells expressing Antibody A with one cell cycle inhibitor compound. As shown in FIG. 3, the tested compound inhibited cell growth without impact on viability, HMW and HM. Specific productivity (qP) was increased.

### Example 4

Various compounds were tested in an initial dosage and time course study to ascertain their effects on cell viability and growth phase arrest substantially as described previously in Example 1. Compounds were evaluated for effect, or lack thereof, on viability, as well as their ability to cause growth arrest. Compounds that maintained at least 80% cell viability at one or more concentrations for the five day duration of this assay were regarded as having relatively little or no cell cytotoxicity. Compounds that exhibited a viable cell density (VCD) of less than 50% of that of the control after five days of culture were judged as possessing growth arrest properties; compounds that that exhibited a viable cell density (VCD) of less than 30% of that of the control were judged as possessing significant growth arrest properties.

Compounds are further evaluated essentially as described in Example 2 herein. Briefly, transfected CHO cells expressing recombinant human monoclonal antibody (mAb; two different cell lines expressing two different mAb, referred to as cell line 1 and cell line 2) are cultured in 24-deep well plates in the presence or absence of the compounds. At specified time points, samples of the cultures are taken and evaluated for viable cell density (VCD; expressed in 10⁶ cells/mL); percent viability (as determined by any method known in the art); titer of recombinant protein (expressed in grams of protein per liter of culture, or g/L, as determined by Protein A affinity HPLC); qP, or specific productivity (expressed in picograms of recombinant protein per cell per day, or pg/c/d). Additional parameters measure include the presence or absence of high molecular weight substances (usually an indicator of protein aggregation; expressed as % of the total protein); and the relative percentage of Mannose 5. For comparison purposes, Compounds A and B, which had been previously tested, were tested again. Viable cell density and percent viability of certain compounds are shown in Table 2 below.

All of the compounds in Tables 2 and 3 below exhibited relatively little or no cell cytotoxicity while exhibiting growth arrest properties, and were evaluated for effects on protein production as described below.

TABLE 2
Viable Cell Density and Percent Viability

| Compund | VCD (e6/mL) | | | | | Viability (%) | | | |
|---|---|---|---|---|---|---|---|---|---|
| (conc., microM) | D 0 | D 1 | D 2 | D 3 | D 4 | D 1 | D 2 | D 3 | D 4 |
| Cell Line 1 | | | | | | | | | |
| control-1 | 10.00 | 11.61 | 15.31 | 17.06 | 23.05 | 95.43 | 92.53 | 89.09 | 89.32 |
| control-2 | 10.00 | 12.32 | 14.68 | 17.06 | 22.59 | 95.91 | 91.53 | 90.59 | 91.93 |
| A (10) | 10.00 | 11.07 | 10.73 | 9.05 | 11.11 | 96.12 | 91.43 | 88.42 | 88.43 |
| B (5) | 10.00 | 10.95 | 9.61 | 10.14 | 12.66 | 94.83 | 85.63 | 82.33 | 78.87 |
| C (10) | 10.00 | 10.72 | 8.77 | 10.83 | 12.18 | 94.37 | 82.97 | 84.46 | 78.70 |
| C (20) | 10.00 | 11.00 | 8.28 | 9.22 | 10.18 | 94.55 | 80.00 | 78.87 | 74.13 |
| D (2.5) | 10.00 | 11.85 | 10.72 | 11.11 | 14.71 | 95.50 | 87.30 | 84.83 | 82.00 |
| D (5) | 10.00 | 11.76 | 10.17 | 9.71 | 11.94 | 95.92 | 86.10 | 79.43 | 76.30 |
| D (10) | 10.00 | 11.35 | 9.65 | 9.31 | 10.04 | 94.69 | 82.20 | 76.69 | 70.89 |
| E (5) | 10.00 | 10.31 | 9.37 | 10.05 | 10.39 | 95.94 | 85.43 | 79.09 | 72.71 |
| E (10) | 10.00 | 9.99 | 9.29 | 9.55 | 10.29 | 94.71 | 83.23 | 76.37 | 72.16 |
| F (1.2) | 10.00 | 11.56 | 10.80 | 11.96 | 14.70 | 96.84 | 90.23 | 89.40 | 88.76 |
| F (2.4) | 10.00 | 11.16 | 10.01 | 10.33 | 12.84 | 95.96 | 89.97 | 88.89 | 86.16 |
| G (5) | 10.00 | 11.74 | 10.58 | 11.02 | 13.45 | 95.55 | 88.10 | 83.82 | 82.62 |
| G (10) | 10.00 | 11.65 | 9.95 | 10.48 | 13.58 | 95.31 | 85.90 | 81.90 | 79.61 |
| H (10) | 10.00 | 11.48 | 12.58 | 13.36 | 16.49 | 95.80 | 89.83 | 87.77 | 86.93 |
| H (20) | 10.00 | 10.69 | 11.08 | 10.91 | 12.99 | 95.60 | 87.70 | 82.95 | 81.36 |
| I (0.625) | 10.00 | 11.17 | 11.98 | 12.81 | 16.91 | 95.26 | 89.90 | 85.53 | 85.47 |
| I (1.25) | 10.00 | 10.89 | 11.16 | 12.25 | 15.33 | 94.78 | 88.73 | 85.80 | 84.19 |
| J (0.625) | 10.00 | 11.40 | 14.02 | 16.48 | 19.37 | 94.94 | 91.87 | 90.31 | 89.98 |
| J (1.25) | 10.00 | 11.54 | 11.25 | 14.06 | 17.54 | 94.24 | 87.53 | 84.29 | 84.81 |
| K (1.25) | 10.00 | 11.87 | 13.04 | 16.39 | 22.55 | 95.72 | 92.13 | 90.36 | 91.21 |
| K (2.5) | 10.00 | 11.77 | 12.07 | 15.31 | 21.06 | 96.38 | 90.97 | 89.25 | 89.80 |
| Cell Line 2 | | | | | | | | | |
| control-1 | 10.00 | 11.08 | 18.97 | 24.80 | 39.88 | 95.41 | 93.47 | 92.56 | 92.90 |
| control-2 | 10.00 | 11.47 | 17.31 | 23.09 | 35.74 | 94.78 | 92.97 | 91.97 | 91.66 |
| A (10) | 10.00 | 9.81 | 9.60 | 8.98 | 10.66 | 94.85 | 91.13 | 87.31 | 87.95 |
| B (5) | 10.00 | 10.88 | 11.15 | 13.45 | 20.16 | 95.11 | 89.60 | 87.50 | 87.46 |
| L (1.2) | 10.00 | 11.76 | 13.63 | 18.56 | 26.40 | 94.88 | 92.63 | 92.09 | 91.17 |
| L (2.4) | 10.00 | 11.43 | 12.95 | 16.19 | 24.27 | 94.24 | 91.47 | 90.68 | 91.43 |

The titer and specific productivity are shown in Table 3 below.

TABLE 3
Titer and Specific Productivity (qP)

| | Titer (g/L) | | | | qP (pg/c/d) | | | |
|---|---|---|---|---|---|---|---|---|
| | D 1 | D 2 | D 3 | D 4 | D 1 | D 2 | D 3 | D 4 |
| Cell Line 1 | | | | | | | | |
| control-1 | 0.45 | 0.69 | 0.98 | 1.08 | 39.04 | 45.27 | 57.26 | 46.86 |
| control-2 | 0.48 | 0.73 | 0.98 | 1.03 | 38.68 | 49.71 | 57.44 | 45.75 |
| A (10) | 0.44 | 0.59 | 0.85 | 0.95 | 39.75 | 55.31 | 93.97 | 85.80 |
| B (5) | 0.47 | 0.70 | 0.96 | 1.19 | 42.90 | 72.84 | 94.99 | 94.23 |
| C (10) | 0.48 | 0.70 | 0.94 | 1.21 | 44.45 | 79.79 | 86.83 | 99.64 |
| C (20) | 0.48 | 0.67 | 0.85 | 1.10 | 43.31 | 80.51 | 91.80 | 108.01 |
| D (2.5) | 0.49 | 0.73 | 0.98 | 1.27 | 41.37 | 67.79 | 87.94 | 86.32 |
| D (5) | 0.49 | 0.70 | 0.87 | 1.11 | 41.95 | 69.17 | 89.92 | 92.69 |
| D (10) | 0.49 | 0.67 | 0.86 | 0.99 | 43.16 | 69.81 | 92.01 | 98.63 |
| E (5) | 0.47 | 0.67 | 0.85 | 0.98 | 45.61 | 71.53 | 84.92 | 94.68 |
| E (10) | 0.48 | 0.66 | 0.83 | 0.96 | 47.72 | 71.04 | 87.30 | 92.97 |
| F (1.2) | 0.49 | 0.73 | 0.96 | 1.13 | 42.39 | 67.31 | 80.01 | 77.09 |
| F (2.4) | 0.50 | 0.75 | 0.97 | 1.18 | 44.50 | 75.27 | 93.88 | 91.87 |
| G (5) | 0.48 | 0.66 | 0.96 | 0.98 | 40.59 | 62.69 | 86.81 | 72.60 |
| G (10) | 0.47 | 0.80 | 0.94 | 1.08 | 40.64 | 80.10 | 89.39 | 79.79 |
| H (10) | 0.47 | 0.69 | 0.94 | 0.92 | 41.24 | 55.12 | 70.60 | 56.01 |
| H (20) | 0.47 | 0.68 | 0.92 | 0.97 | 44.27 | 61.66 | 83.98 | 74.65 |
| I (0.625) | 0.46 | 0.66 | 0.89 | 1.07 | 41.50 | 55.10 | 69.73 | 63.09 |
| I (1.25) | 0.46 | 0.66 | 0.89 | 1.06 | 42.55 | 58.82 | 72.68 | 69.35 |
| J (0.625) | 0.45 | 0.68 | 0.88 | 0.99 | 39.77 | 48.50 | 53.61 | 51.10 |
| J (1.25) | 0.44 | 0.64 | 0.86 | 1.08 | 38.41 | 57.19 | 61.40 | 61.77 |
| K (1.25) | 0.48 | 0.72 | 1.01 | 1.07 | 40.15 | 54.94 | 61.61 | 47.60 |
| K (2.5) | 0.48 | 0.72 | 1.05 | 1.22 | 40.78 | 59.38 | 68.37 | 58.10 |
| Cell Line 2 | | | | | | | | |
| control-1 | 0.26 | 0.49 | 0.63 | 0.82 | 23.78 | 26.00 | 25.27 | 20.48 |
| control-2 | 0.25 | 0.42 | 0.49 | 0.64 | 22.08 | 24.45 | 21.37 | 17.91 |
| A (10) | 0.23 | 0.30 | 0.30 | 0.43 | 23.45 | 31.24 | 33.04 | 40.64 |
| B (5) | 0.29 | 0.49 | 0.62 | 0.89 | 26.34 | 43.95 | 46.35 | 43.97 |
| L (1.2) | 0.28 | 0.49 | 0.58 | 0.70 | 23.81 | 35.94 | 31.42 | 26.64 |
| L (2.4) | 0.28 | 0.47 | 0.60 | 0.76 | 24.20 | 36.29 | 37.05 | 31.18 |

These results indicate that several compounds not only induce growth arrest, but also have a major (>200%) increase in specific productivity (qP). Compounds D, F, and G have minimal effects on viability while achieving reasonably sustained growth arrest. Compounds B, C, and E also increase qP by a factor of greater than 200% but have greater (but still modest) decreases in viability; these compounds appear to produce more sustained growth arrest. Moreover, at least one additional compound demonstrated a relative lack of cell cytotoxicity and a significant increase in growth arrest, as shown in Table 4 below.

TABLE 4
Effects of Compound on Cell Culture Properties

| Cmp'nd | | | | | | | | | |
|---|---|---|---|---|---|---|---|---|---|
| (conc., microM) | VCD (e6/mL) | | | | | Viability (%) | | | |
| Cell Line | D 0 | D 1 | D 2 | D 3 | D 4 | D 1 | D 2 | D 3 | D 4 |
| Control | 0.5000 | 0.4850 | 0.8658 | 1.6649 | 2.4784 | Control | 97.90 | 97.67 | 96.58 |
| A (5) | 0.5000 | 0.3826 | 0.4289 | 0.6095 | 0.9224 | A 5 | 97.90 | 97.77 | 97.09 |
| A (10) | 0.5000 | 0.3721 | 0.3059 | 0.4855 | 0.6155 | A 10 | 97.90 | 97.88 | 94.50 |
| M (2.5) | 0.5000 | 0.4656 | 0.6879 | 1.1916 | 2.1883 | M 2.5 | 97.90 | 97.65 | 96.47 |
| M (5) | 0.5000 | 0.4129 | 0.5439 | 0.8760 | 1.4543 | M 5 | 97.90 | 97.67 | 95.90 |
| M (10) | 0.5000 | 0.4204 | 0.4984 | 0.7601 | 1.2297 | M 10 | 97.90 | 97.06 | 95.85 |
| M (20) | 0.5000 | 0.3915 | 0.4101 | 0.5348 | 0.8023 | M 20 | 97.90 | 97.31 | 96.04 |

These results demonstrate that Compound M exhibits cytotoxicity and growth arrest properties that are very similar, at similar concentrations, to the properties of Compound A. Based on these results, and on structural similarity and results in a CDK4 assay and cell proliferation assay (substantially as described in Examples 6 and 7), Compound M will also have a major (>200%) increase in specific productivity (qP) of recombinant protein (i.e., antigen binding protein or antibody) by mammalian cells such as CHO cells.

### Example 5

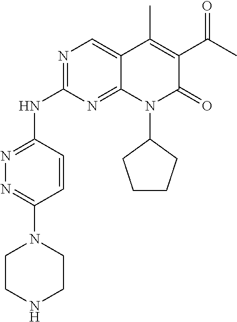 [SMILES: CC(=O)c1c(C)c2cnc(Nc3ccc(N4CCNCC4)nn3)nc2n(C2CCCC2)c1=O]

### 6-acetyl-8-cyclopentyl-5-methyl-2-((6-(piperazin-1-yl)pyridazin-3-yl)amino)pyrido[2,3-d]pyrimidin-7(8H)-one

### Synthesis of 6-acetyl-2-amino-8-cyclopentyl-5-methylpyrido[2,3-d]pyrimidin-7(8H)-one

6-Acetyl-2-amino-8-cyclopentyl-5-methylpyrido[2,3-d]pyrimidin-7(8H)-one was prepared as described in Example 135 of U.S. Pat. No. 7,456,168. ¹H NMR (400 MHz, CD₃OD) δ 1.63-1.70 (2H, m), 1.81-1.87 (2H, m), 2.02-2.14 (2H, m), 2.24-2.32 (2H, m), 2.32 (3H, s), 2.46 (3H, s), 5.97 (1H, m), 8.73 (1H, s); MS (ESI) m/z: 287.0 [M+H]⁺.

### Synthesis of tert-butyl 4-(6-chloropyridazin-3-yl)piperazine-1-carboxylate

To a solution of 3,6-dichloropyridazine (Sigma-Aldrich, St. Louis, Mo.) (57.3 g, 385 mmol) in 1,4-dioxane (250 mL) were added tert-butyl piperazine-1-carboxylate (Sigma-Aldrich) (71.6 g, 358 mmol) and N,N-diisopropylethylamine (66.9 mL, 385 mmol). The mixture was stirred overnight at 80° C. then concentrated under reduced pressure. The residue was dissolved in ethyl acetate (3 L) and washed with 10% citric acid, water, and brine. The organic layer was concentrated and the residue was re-crystallized in ethyl acetate to provide tert-butyl 4-(6-chloropyridazin-3-yl)piperazine-1-carboxylate as an off-white solid (101 g, 88% yield). ¹H NMR (400 MHz, CDCl₃) δ ppm 1.25 (9H, s), 3.26-3.47 (8H, m), 6.67 (1H, d, J=9.6 Hz), 7.00 (1H, d, J=9.6 Hz); MS (ESI) m/z: 299.0 [M+H]⁺.

### Synthesis of tert-butyl 4-(6-((6-acetyl-8-cyclopentyl-5-methyl-7-oxo-7,8-dihydropyrido[2,3-d]pyrimidin-2-yl)amino)pyridazin-3-yl)piperazine-1-carboxylate

To a solution of acetyl-2-amino-8-cyclopentyl-5-methylpyrido[2,3-d]pyrimidin-7(8H)-one (114 mg, 0.40 mmol) in 1,4-dioxane (4 mL) were added tert-butyl 4-(6-chloropyridazin-3-yl)piperazine-1-carboxylate (143 mg, 0.48 mmol), tris(dibenylideneacetone)dipalladium (0) (18.4 mg, 0.02 mmol), 4,5-bis(diphenylphosphino)-9,9-dimethylxanthene (35 mg, 0.06 mmol), sodium t-butoxide (58 mg, 0.60 mmol). The mixture thus obtained was heated for 1 h at 150° C. under microwave irradiation. The reaction mixture was passed through short pack silica gel column and was concentrated to give a crude product as a yellow solid (153 mg, purity: 50-60%) which was used in next step without any further purification. MS (ESI) m/z: 549.3 [M+H]⁺.

### Synthesis of 6-acetyl-8-cyclopentyl-5-methyl-2-((6-(piperazin-1-yl)pyridazin-3-yl)amino)pyrido[2,3-d]pyrimidin-7(8H)-one

A solution of tert-butyl 4-(6-((6-acetyl-8-cyclopentyl-5-methyl-7-oxo-7,8-dihydropyrido[2,3-d]pyrimidin-2-yl)amino)pyridazin-3-yl)piperazine-1-carboxylate (153 mg, purity: 50-60%) in trifluoroacitic acid/dichloromethane (1:1, 4 mL) was stirred for 30 min at room temperature. The reaction mixture was concentrated and the residue was purified by flash chromatography on silica gel, eluting with CH₂Cl₂/MeOH/NH₄OH (200:10:1) to give 6-acetyl-8-cyclopentyl-5-methyl-2-((6-(piperazin-1-yl)pyridazin-3-yl)amino)pyrido[2,3-d]pyrimidin-7(8H)-one as a yellow solid (41 mg, 61% yield). ¹H NMR (500 MHz, CDCl₃) δ ppm 1.66-1.77 (3H, m), 1.80-1.95 (2H, m), 2.00-2.16 (2H, m), 2.30-2.42 (2H, m), 2.38 (3H, s), 2.56 (3H, s), 3.02-3.07 (4H, m), 3.57-3.62 (4H, m), 5.85 (1H, m), 7.04 (1H, d, J=9.8 Hz), 8.11 (1H, br. s.), 8.33 (1H, d, J=9.8 Hz), 8.79 (1H, s); MS (ESI) m/z: 449.2 [M+H]⁺.

### Example 6

### CDK4 Assay

The CDK4 inhibitory activity of the cell cycle inhibitors was determined with a filtration kinase assay. The compounds, kinase and substrate diluted in the kinase buffer (20 mM Tris, pH7.4, 50 mM NaCl, 1 mM DTT, 0.1% BSA) were sequentially added to a 96-well Multiscreen HTS filtration plate (Millipore). The final 100 μL reaction mixture in each well contained 0.3 μg of CDK4/Cyclin D1 (Cell Signaling Technology), 1 μg of Rb fragment (aa773-928, Millipore) for the CDK4 assay and 1 μCi of [³³P]-ATP. The mixture was incubated at room temperature for 1 hour. The proteins in the reaction were then precipitated and washed with cold TCA solution using an aspiration/filtration vacuum system. The plates were dried at room temperature, and the retained radioactivity was measured by scintillation counting. IC₅₀ values of the compounds in the above assays were determined by non-linear regression analysis using Prism (GraphPad Software).

TABLE 6
Activity in a CDK4 Assay

| Compound | CDK4 IC₅₀ microM |
|---|---|
| B | 0.00275 |
| C | 0.003171 |
| D | 0.00186 |
| E | 0.00445 |
| F | 0.03353 |
| L | 0.001403 |
| M | 0.015 |

### Example 7

### Cell Assay

The cell proliferation inhibition potency of the cell cycle inhibitors was determined by using a [¹⁴C]-thymidine incorporation assay. Exponentially growing cells (Colo205) were seeded in a 96-well Cytostar T plate (GE Healthcare Biosciences) at a density of 5×10³ cells/well and incubated overnight. Serially diluted compounds and 0.1 μCi of [¹⁴C]-thymidine (GE Healthcare Biosciences) were added to each well on the following day. After 72 hour incubation, isotope incorporation was determined with a β plate counter (Wallac). IC₅₀ values of the compounds in the above assays were determined by non-linear regression analysis using Prism (GraphPad Software).

TABLE 7
Activity in a Cell Proliferation Inhibition Assay

| Compound | Cell Assay IC₅₀ microM |
|---|---|
| B | 0.014844 |
| C | 0.017054 |
| D | 0.02992 |
| E | 0.0384 |
| F | 0.0347 |
| L | 0.0235 |
| M | 0.09195 |

1. A method of increasing specific productivity in a mammalian cell culture expressing a recombinant protein comprising
establishing a mammalian cell culture in a culture medium;
inducing cell growth-arrest by contacting the cell culture with a culture medium comprising a cell cycle inhibitor that has an IC₅₀ in a CDK4 enzyme inhibition assay of less than about 20 nM; and
maintaining the cell culture in a growth-arrested state by contacting the culture with a culture medium comprising a cell cycle inhibitor.

2. A method of increasing recombinant protein production in a mammalian cell culture expressing a recombinant protein comprising
establishing a mammalian cell culture in a culture medium;
inducing cell growth-arrest by contacting the cell culture with a culture medium comprising a cell cycle inhibitor that has an IC₅₀ in a CDK4 enzyme inhibition assay of less than about 20 nM; and
maintaining the cell culture in a growth-arrested state by contacting the culture with a culture medium comprising a cell cycle inhibitor.

3. A method of limiting a mammalian cell culture expressing a recombinant protein at a desired packed cell volume comprising
establishing a mammalian cell culture in a culture medium;
inducing cell growth-arrest by contacting the cell culture with a culture medium comprising a cell cycle inhibitor that has an IC₅₀ in a CDK4 enzyme inhibition assay of less than about 20 nM; and
maintaining the cell culture in a growth-arrested state by contacting the culture with a culture medium comprising a cell cycle inhibitor.

4. The method of claim 1, wherein the cell culture is contacted with culture medium comprising a cell cycle inhibitor on or before day 3 of the culture.

5. The method claim 1, wherein induction of cell growth-arrest takes place prior to a production phase.

6. The method claim 1, wherein induction of cell growth-arrest takes place during a production phase.

7. The method of claim 1, wherein the cell cycle inhibitor has an IC₅₀ in a CDK4 cell assay of less than about 100 nM.

8. The method of claim 1, wherein the cell cycle inhibitor is selected from the group consisting of: N-(6-(4-(dimethylamino)-1-piperidinyl)-3-pyridazinyl)-9-(trans-4-methylcyclohexyl)-9H-pyrido[4′,3′,:4,5]pyrrolo[2,3-d]pyrimidin-2-amine, 9-cyclopentyl-N-(6-(4-(dimethylamino)piperidin-1-yl)pyridazin-3-yl)-9H-pyrido[4′,3′:4,5]pyrrolo[2,3-d]pyrimidin-2-amine, 9-(4-methylcyclohexyl)-N-(6-(3-methylpiperazin-1-yl)pyridazin-3-yl)-9H-pyrido[4′,3′:4,5]pyrrolo[2,3-d]pyrimidin-2-amine, 8-(6-((9-(4-methylcyclohexyl)-9H-pyrido[4′,3′:4,5]pyrrolo[2,3-d]pyrimidin-2-yl)amino)pyridazin-3-0)-2-thia-8-azaspiro[4.5]decane 2,2-dioxide, N-(6-(4-(dimethylamino)piperidin-1-yl)pyridin-3-0)-4,4-dimethylspiro[cyclohex[2]ene-1,9′-pyrido[4′,3′:3,4]cyclopenta[1,2-d]pyrimidin]-2′-amine
and the salts, esters, and amides, thereof.

9.-12. (canceled)

13. The method claim 1, wherein the cell culture is grown by a method selected from the group consisting of batch culture, fed-batch culture, perfusion culture, and combinations thereof.

14. The method claim 1, further comprising a temperature shift from 36° C. to 31° C.

15. The method of claim 1, wherein the method further comprises limitation of a key nutrient in the culture medium.

16.-20. (canceled)

21. The method claim 1, wherein the concentration of cell cycle inhibitor in the culture medium is between 0.5 and 5 mM.

22. The method claim 1, wherein the concentration of cell cycle inhibitor in the culture medium is between 1 and 4.0 mM.

23. The method claim 1, wherein the concentration of cell cycle inhibitor in the culture medium is between 2 and 3.0 mM.

24. The method claim 1, wherein the concentration of cell cycle inhibitor in the culture medium is between 2.5 and 5 mM.

25. The method claim 1, wherein the concentration of cell cycle inhibitor in the culture medium is between 3.5 and 5 mM.

26. The method of claim 1, wherein the packed cell volume during a production phase is less than or equal to 35%

27. The method of claim 1, wherein the packed cell volume is less than or equal to 30%.

28. The method of claim 1, wherein the viable cell density of the mammalian cell culture at a packed cell volume less than or equal to 35% is 10×10⁶ viable cells/ml to 80×10⁶ viable cells/ml.

29. The method claim 1, wherein the viable cell density of the mammalian cell culture is 20×10⁶ viable cells/ml to 30×10⁶ viable cells/ml.

30.-44. (canceled)

45. The method of claim 1, wherein the mammalian cells are Chinese Hamster Ovary (CHO) cells.

46. The method of claim 1, wherein the recombinant protein is selected from the group consisting of a human antibody, a humanized antibody, a chimeric antibody, a recombinant fusion protein, or a cytokine.

47. The method of claim 46, further comprising a step of harvesting the recombinant protein produced by the cell culture.

48. The method of claim 47, wherein the recombinant protein produced by the cell culture is purified and formulated in a pharmaceutically acceptable formulation.

49. (canceled)
